(19) United States
(12) Patent Application Publication
(10) Pub. No.: US 2020/0077971 A1
(43) Pub. Date: Mar. 12, 2020

(54) RADIATION IMAGING SYSTEM

(71) Applicant: Konica Minolta, Inc., Tokyo (JP)

(72) Inventors: Masahiro KUWATA, Tokyo (JP); Ikuma OTA, Tokyo (JP); Tomoyasu YOKOYAMA, Tsurugashima-shi (JP); Sho NOJI, Tokyo (JP); Takuya YAMAMURA, Tokyo (JP); Takafumi MATSUO, Tokyo (JP); Hidetake TEZUKA, Tokyo (JP)

(21) Appl. No.: 16/554,113

(22) Filed: Aug. 28, 2019

(30) Foreign Application Priority Data
Sep. 10, 2018 (JP) 2018-168415

(51) Int. Cl. A61B 6/00

(52) U.S. Cl. CPC A61B 6/588; A61B 6/4405; A61B 6/547

(57) ABSTRACT

There is provided a radiation imaging system including a radiation emitting apparatus 3 having a radiation source 34 that generates radiation, a radiation imaging apparatus 100B that receives radiation and generates radiation image data, and a hardware processor. The hardware processor detects height of the radiation source 34 and height of the radiation imaging apparatus 100B. The hardware processor further calculates a distance from a focal point of the radiation generated by the radiation source 34 to the radiation imaging apparatus 100B based on the height of the radiation source 34 and the height of the radiation imaging apparatus 100B, and causes a display to display the calculated distance.

### BACKGROUND

### Technological field

The present invention relates to a radiation imaging system.

### Description of the Related art

In radiation imaging using a radiation imaging system including a radiation source and a radiation imaging apparatus, it is necessary to manually adjust the distance between the focus of the radiation in the radiation source and the radiation imaging apparatus (Source Image receptor Distance, hereinafter referred to as SID). When this radiation imaging system is of a stationary type installed in a dedicated room, the SID can be adjusted relatively easily since the movable range of each device constituting the radiation imaging system is relatively limited by a slide rail and the like.

However, the movable range is not limited very much when the radiation imaging system is of a movable type configured to be movable with wheels or the like, and it was necessary to visually judge the position, angle, and SID of each device. Therefore, it was difficult to manually adjust the SID to a standard imaging distance predetermined on the imaging technique or the grid used for imaging.

Furthermore, even in an imaging room where the stationary radiation imaging system is installed, there is a problem that, when the user designates an incorrect SID, imaging may be performed with an unintended SID.

Furthermore, even in the imaging room where the stationary radiation imaging system is installed, the user may manually adjust the body position, angle, SID, and the like in some cases in order to perform imaging at a desired body position and angle using a portable radiation imaging apparatus, instead of using an imaging table where the radiation imaging apparatus is installed. Even in such a case, it is necessary to perform imaging with a predetermined SID.

A grid put on the radiation incident surface of the radiation imaging apparatus transmits radiation emitted radially from the radiation source which is arranged at a position away from the grid by a predetermined SID, so that the radiation entering on the center of the grid is perpendicular to the radiation incident surface. Therefore, the grid shields the radiation even in a case where the distance between the radiation source and the center of the grid is equal to the corresponding SID. As a result, there is a problem that the dose of radiation reaching the radiation imaging apparatus is reduced if the grid is slightly tilted from the plane perpendicular to the radiation emitted toward the center of the grid, and that the image quality is deteriorated.

Even in imaging without a grid, a magnification of the subject in imaging changes as the SID changes. Therefore, the actual magnification in imaging is different from the expected value if, for example, the actual SID is different from a determined SID. This may cause a problem that the size of a specific portion or a specific tumor of a subject is incorrectly calculated from the image. This is particularly problematic in evaluation of temporal changes in the size of a specific portion or a specific tumor by imaging multiple times at specific time intervals.

In order to overcome such various problems, conventionally, techniques as described in JP2013-523396A, JP2014-507247A, and JP2017-060544A have been proposed.

Specifically, JP2013-523396A discloses a technique for detecting information on angle data, SID, and the outline of a receiver using an electromagnetic field sensor.

Further, JP2014-507247A discloses a technique for detecting and adjusting the SID, a position of the emission field relative to the imaging apparatus 100B, and the like, using at least two magnetic sensors disposed at different angles from each other.

Further, JP 2017-060544 A discloses a technique including arranging multiple detectors so as to partially overlap one another, taking an image of a marker in the overlapping portion, and calculating an SID based on the enlargement ratio of a marker M taken by a first detector relative to the marker taken by another detector which is closer to the radiation source than the first detector.

In the radiation imaging system described in JP2013-523396A and JP2014-507247A, it is necessary to use magnetism for measurement of SID and the like. Therefore, there is a problem that it is difficult to use in combination with a device susceptible to the magnetic field. Also, there is a problem that the SID and the like is measured inaccurately due to noise including the magnetic field from a magnet worn by the subject for the purpose of promoting blood circulation etc.

There is also a problem that the exposure dose to the subject is increased in the radiation imaging system that requires to perform radiation imaging for measuring SID separately from radiation imaging for acquiring diagnostic images described in JP 2017-060544 A.

### SUMMARY

An object of the present invention is to make it possible to easily adjust SID in a radiation imaging system including a radiation emitting apparatus and a radiation imaging apparatus without using magnetism or increasing the exposure dose of the subject.

To achieve at least one of the abovementioned objects, according to a first aspect of the present invention, a radiation imaging system reflecting one aspect of the present invention includes:

a radiation emitting apparatus having a radiation source that generates radiation;

a radiation imaging apparatus that receives radiation and generates radiation image data; and

a hardware processor, wherein

the hardware processor
detects height of the radiation source,
detects height of the radiation imaging apparatus,
calculates a distance from a focal point of the radiation generated by the radiation source to the radiation imaging apparatus based on the height of the radiation source and the height of the radiation imaging apparatus, and
causes a display to display the calculated distance.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram showing a configuration of a radiation imaging system according to a first embodiment.

FIG. 2 is a block diagram showing a configuration of a radiation imaging apparatus provided in the radiation imaging system of FIG. 1.

FIG. 3 is a perspective view of a radiation imaging apparatus provided in the radiation imaging system of FIG. 1.

FIG. 4 is a side view showing an example of the configuration of the radiation imaging system of FIG. 1.

FIG. 5 is a side view and a plan view of a radiation imaging apparatus provided in a radiation imaging system according to a modification of the first embodiment.

FIG. 6 is a ladder chart showing a flow of imaging using the radiation imaging system of FIG. 1

FIG. 7 is a block diagram showing a configuration of a radiation imaging system according to a second embodiment A.

FIG. 8 is a block diagram showing a configuration of a body motion detecting apparatus provided in the radiation imaging system of FIG. 7.

FIG. 9 is an example of a specific portion designated by a body motion detecting apparatus of FIG. 8.

FIG. 10 is a block diagram showing a configuration of a radiation imaging system according to a second embodiment B.

FIG. 11 is a side view of a radiation imaging system according to an example.

FIG. 12 is a side view showing a part of a radiation imaging system according to an example.

FIG. 13 is a graph for explaining a principle of the example.

FIG. 14 is a side view showing a part of a radiation imaging system according to an example.

FIG. 15 is a perspective view showing a part of a radiation imaging system according to an example.

FIGS. 16A and 16B are side views showing a part of a radiation imaging system according to an example.

FIG. 17 is a side view showing a part of a radiation imaging system according to the example.

FIG. 18A is a plan view showing a radiation imaging apparatus provided in the radiation imaging system according to the example.

FIG. 18B is a side view showing the radiation imaging apparatus of FIG. 18A.

FIG. 18C is a radiographic image taken by the radiation imaging system according to the example.

FIG. 19A is a plan view showing a radiation imaging apparatus provided in the radiation imaging system according to the example.

FIG. 19B is a side view showing the radiation imaging apparatus of FIG. 19A.

FIG. 19C is a radiographic image taken by the radiation imaging system according to the example.

FIG. 20 is a conceptual diagram for explaining a principle of a modification of the example.

FIGS. 21A and 21B are side views showing a radiation imaging apparatus provided in the radiation imaging system according to the example.

FIG. 22 is a conceptual diagram for explaining a principle of a radiation imaging system according to an example.

FIG. 23 is a perspective view showing a radiation imaging apparatus provided in a radiation imaging system according to an example.

FIG. 24 is a plan view showing a radiation imaging apparatus provided in a radiation imaging system according to an example.

FIG. 25 is a plan view showing a part of a radiation imaging apparatus provided in a radiation imaging system according to an example.

FIG. 26A is a plan view showing a radiation imaging apparatus provided in a radiation imaging system according to an example.

FIG. 26B is a side view of the radiation imaging apparatus of FIG. 26A.

FIG. 27 is a perspective view for explaining an imaging method using a radiation imaging system according to an example.

FIGS. 28A and 28B are a plan view and a cross-sectional view of a marker M provided in a radiation imaging system according to an example.

FIG. 29 is a side view showing a radiation imaging system according to an example.

FIG. 30 is a side view showing a radiation imaging system according to an example.

FIG. 31A and FIG. 31B are perspective views for explaining an imaging method using a radiation imaging system according to an example.

FIGS. 32A and 32B are plan views showing a radiation imaging apparatus provided in the radiation imaging system according to the example.

FIGS. 33A and 33B are side views showing a radiation imaging apparatus provided in the radiation imaging system according to the example.

FIG. 34 is a perspective view for explaining an imaging method using a radiation imaging system according to an example.

FIG. 35 is a conceptual diagram for explaining a principle of an example.

FIG. 36 is a flowchart of processing executed by a radiation imaging system according to an example.

FIG. 37 is a flowchart of processing executed by a radiation imaging system according to a modification of the example.

FIGS. 38A to 38C are diagrams for explaining an imaging method using a radiation imaging system according to an example.

FIG. 39 is a perspective view of a radiation imaging system according to an example.

FIG. 40 is a block diagram showing a configuration of the radiation imaging system of FIG. 37.

FIGS. 41A to 41E are diagrams for explaining an imaging method using a radiation imaging system according to an example.

FIGS. 42A to 42D are diagrams for explaining an imaging method using a radiation imaging system according to an example.

FIG. 43A is a diagram for explaining an imaging method using a radiation imaging system according to an example.

FIG. 43B is a front view of a display provided in the system of FIG. 43A.

FIGS. 44A and 44B are diagrams for explaining an imaging method using a radiation imaging system according to an example.

FIG. 45 is a diagram for explaining an imaging method using a radiation imaging system according to an example.

FIG. 46A is a diagram for explaining an imaging method using a radiation imaging system according to an example.

FIG. 46B is a graph displayed on a display included in the system of FIG. 46A.

FIG. 47 is a diagram for explaining an imaging method using a radiation imaging system according to an example.

FIG. 48A is a diagram for explaining an example of a method for setting a specific region in imaging using a radiation imaging system according to an example.

FIGS. 48B and 48C are graphs showing changes in body motion amount with time in the specific region set in FIG. 48A.

FIG. 49A is a diagram for explaining an imaging method using a radiation imaging system according to an example.

FIG. 49B is an example of a template used in imaging using the system of FIG. 49A.

FIGS. 50A and 50B are diagrams for explaining an imaging method using a radiation imaging system according to an example.

FIG. 51 is a diagram for explaining an imaging method using a radiation imaging system according to an example.

FIG. 52A is a diagram showing an example of a method of setting a region of interest in imaging using a radiation imaging system according to an example.

FIG. 52B is a graph showing change over time in a density value in the region of interest set in FIG. 52A.

FIG. 53 is a diagram for explaining an imaging method using a radiation imaging system according to an example.

FIG. 54 is a diagram for explaining an imaging method using a radiation imaging system according to an example.

FIG. 55 is a diagram for explaining an imaging method using a radiation imaging system according to an example.

FIG. 56 is a flowchart of processing executed by a radiation imaging system according to a modification of the example.

FIG. 57 is a block diagram showing a configuration of a radiation imaging system according to an example.

FIG. 58 is a timing chart showing operation of a part of a radiation imaging system according to an example.

FIG. 59A is a side view of a partial configuration included in a radiation imaging system according to an example.

FIG. 59B is a perspective view of another partial configuration of a radiation imaging system according to an example.

FIG. 60 is a flowchart of processing executed by a radiation imaging system according to an example.

FIG. 61 is a side view showing a radiation imaging system according to an example.

FIG. 62A is a side view of a partial configuration included in a radiation imaging system according to an example.

FIGS. 62B and 62C are perspective views of a partial configuration of a radiation imaging system according to an example.

FIG. 63 is a side view of a partial configuration of a radiation imaging system according to an example.

FIG. 64 is a flowchart of processing executed by a radiation imaging system according to an example.

FIG. 65A is a diagram for explaining an imaging method using a radiation imaging system according to an example.

FIG. 65B is a frame image taken using the system of FIG. 65A.

FIG. 66 is a diagram for explaining an imaging method using a radiation imaging system according to a modification of the example.

FIG. 67 is a diagram for explaining an imaging method using a radiation imaging system according to an example.

FIG. 68 is a side view of a radiation imaging system according to an example.

FIG. 69 is a block diagram showing a configuration of the radiation imaging system shown in FIG. 66.

FIG. 70 is a side view showing a radiation imaging system according to an example.

FIG. 71A is a diagram for explaining a conventional imaging method where normal imaging is performed.

FIG. 71B is a diagram for explaining a conventional imaging method where normal imaging is not performed.

FIG. 72A is a front view of an imaging apparatus included in a radiation imaging system according to an example.

FIGS. 72B and 72C are radiographic images taken using the imaging apparatus of FIG. 72A.

FIG. 73 is a diagram for explaining an imaging method using a radiation imaging system according to an example.

FIGS. 74A to 74D are side views showing a part of a radiation imaging system according to an example.

FIG. 75 is a diagram for explaining an imaging method using a radiation imaging system according to an example.

FIG. 76A is a diagram for explaining an imaging method using a radiation imaging system according to an example.

FIGS. 76B and 76C are examples of how a marker is captured before imaging with the imaging method of FIG. 76A.

FIGS. 77A and 77B are plan views of markers used in imaging using the radiation imaging system of FIGS. 74A to 74D.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

First of all, a first embodiment will be described with reference to the drawings.

### [Configuration of Radiation Imaging System]

First, an outline of the radiation imaging system 100 according to the first embodiment will be described. FIG. 1 is a block diagram of a radiation imaging system 100 of the present embodiment.

As shown in FIG. 1, the radiation imaging system 100 according to the present embodiment is configured to include a system main body 100A and one or more radiation imaging apparatus (hereinafter, imaging apparatus(es) 100B).

The radiation imaging system 100 can be connected by wire or wirelessly to an image analysis device, a radiology information system (RIS), a picture archiving and communication system (PACS), and the like (not shown in the drawings).

The radiation imaging system 100 according to the present embodiment may be, for example, a mobile system for radiation imaging by visiting a subject S (subject) who has difficulty in moving. In that case, the system main body 100A is preferably configured as a movable vehicle having wheels la, and the radiation imaging apparatus 100B is preferably of a panel type (of a portable type).

Hereinafter, the radiation imaging system 100 configured as a mobile type will be described as an example. Therefore, in the following description, the system main body 100A is referred to as a movable vehicle 100A.

The radiation imaging system 100 according to the present embodiment can also be installed and used, for example, in a photographing room of a hospital.

The movable vehicle 100A is configured to be able to set various imaging conditions, to irradiate the subject S and the imaging apparatus 100B behind the subject S with radiation, to perform predetermined image processing on image data input by the imaging apparatus 100B, to display an image, and to output the image data to the outside.

The movable vehicle 100A will be described in detail later.

The imaging apparatus 100B is communicably connected by wire or wirelessly to the movable vehicle 100A.

The imaging apparatus 100B can generate radiographic image data by receiving radiation from the outside (movable vehicle 100A).

The imaging apparatus 100B will be also described in detail later.

The radiation imaging system 100 according to the present embodiment configured as described above can take at least one of a still image and serial images of the subject S by irradiating the subject S in front of the imaging apparatus 100B with radiation emitted from the movable vehicle 100A.

In serial imaging in the present embodiment, a series of multiple images are acquired by repeatedly taking an image of the subject Sin response to one imaging operation (depression of the exposure switch 31a described later), that is, by accumulation of charges and reading of signal value repeated several times in a short time by the imaging apparatus 100B (repeated generation of image data of the subject S) while the movable vehicle 100A repeatedly irradiates the subject S with radiation

Hereinafter, a series of multiple images acquired by serial imaging is referred to as a dynamic image, and individual images constituting the dynamic image are referred to as frame images.

### [Electric Configuration of Movable Vehicle]

Next, an electrical configuration of the movable vehicle 100A constituting the radiation imaging system 100 will be described.

The movable vehicle 100A is configured to include, in addition to the housing 1 provided with wheels la, an imaging controller 2, a radiation emitting apparatus 3, a console 4, a power supply unit 5, and the like.

The imaging controller 2 is configured to include a central processing unit (CPU), a random access memory (RAM), a storage, a crystal oscillator, and the like (not shown in the drawings).

The CPU of the imaging controller 2 reads out a system program and various processing programs stored in the storage, loads them into the RAM, and controls the operation of each part of the movable vehicle 100A according to the loaded programs.

The storage of the imaging controller 2 is composed of a nonvolatile semiconductor memory, a hard disk, etc., and stores various programs executed by the imaging controller 2 and parameters necessary for execution of processing using the programs. The storage of the imaging controller 2 can also store data such as processing results.

The communication unit 21 includes a wired communication interface (hereinafter referred to as a wired communication IF) 21a in which an communication cable extending from the imaging apparatus 100B is inserted for performing wired communication with the imaging apparatus 100B, or a wireless interface (hereinafter referred to as a wireless communication IF) 21b for performing wireless communication with imaging apparatus 100B. The communication unit 21 can switch the connection method to a wired one or a wireless one based on the control signal from the CPU.

The radiation emitting apparatus 3 includes an operation unit 31, a radiation controller 32, a high pressure generator 33, a radiation source (tube) 34, a collimator 35, and the like.

The operation unit 31 includes a button or a touch panel which can be operated by a user, detects operation by the user (kind of the pressed button, contact position of a finger or a touch pen, etc.), and outputs the detected operation to the radiation controller 32 as operation information.

In addition, an exposure switch 31a that allows the user to give command to emit radiation X is connected to the operation unit 31. The exposure switch 31a is a two-step switch.

Then, the operation unit 31 detects the number of steps of the operations performed on the exposure switch 31a, and outputs it to the radiation controller 32 as exposure switch information.

The exposure switch 31a may be connected to the movable vehicle 100A in by wire or wirelessly so as to perform remote operation. In this way, the user can control exposure of the radiation from a place away from the radiation emitting apparatus 3 of the movable vehicle 100A.

The radiation controller 32 can set various imaging conditions (conditions related to the subject S such as a region to be imaged, physical size, etc., and conditions related to emission of radiation such as a tube voltage, tube current, emission time, and product of current and time) according to the operation information from the operation unit 31.

Further, the radiation controller 32 sends control information for instructing the high voltage generator 33 to start voltage application (emission of radiation) in response to receiving the exposure switch information.

In response to receiving the control signal from the radiation controller 32, the high voltage generator 33 applies a voltage according to conditions related to emission of radiation set in advance to the radiation source 34.

In some cases, imaging may be performed not in an imaging room where radiation is prevented from leaking out of the room, but in a ward where the subject S is hospitalized. Therefore, radiation imaging may be performed by outputting weaker radiation in the radiation emitting apparatus 3 of the movable vehicle 100A than in the radiation emitting apparatus 3 fixed in the imaging room. In this case, the high voltage generator 33 may be configured to operate with lower electric power than the one fixed in the imaging room.

The radiation source 34 includes, for example, a rotating anode, a filament, etc. (not shown in the drawings). When the high voltage generator 33 applies a voltage to the radiation source 34, the filament irradiates the rotating anode with the electron beam depending on the voltage, and the rotating anode generates a dose of radiation X corresponding to the intensity of the electron beam.

Specifically, the radiation source 34 continuously emits radiation when a high voltage generator continuously applies a voltage to the radiation source 34, and the radiation source 34 emits pulses of radiation when the high pressure generator applies pulses of voltage to the radiation source 34.

That is, the radiation emitting apparatus 3 of the present embodiment can perform any of imaging of still images, serial imaging of a continuous emission type, and serial imaging of a pulse emission type.

The collimator 35 is arranged on the emission port (on the light path of the radiation X) of the radiation source 34.

The collimator 35 has, for example, a plurality of shielding plates each arranged on the upper, lower, left, and right sides of the optical path of the radiation X to form a rectangular opening, and an adjustment mechanism (not shown in the drawings) for moving the shielding plates. The collimator 35 can adjust the radiation emission field by the adjustment mechanism that changes the position of the respective shielding plates according to the control signal from the radiation controller 32.

The console 4 is configured as a computer or a dedicated control device, and includes a controller, storage, an operation unit, and the like (not shown in the drawings).

When the console 4 receives image data from the imaging apparatus 100B, the console 4 performs, automatically or in response to the user's predetermined operation, an imaging process such as a predetermined correction process on the image data to generate a processed image.

Here, the “imaging process” refers to a process of adjusting the legibility of an image by changing its brightness, density, or the like.

The console 4 also judges the system configuration (specifically, the connection method) between the console 4 itself and the image analysis device.

Further, according to the judgement result of the system configuration, the console 4 can generate compressed image data by compression of the processed image data, generate thinned image data by thinning out part of the frame image data from the processed image data, and the like.

Further, the console 4 can send at least one of the processed image data, compressed image data, and thinned image data to the image analysis device via the communication unit 42.

A display 41 is configured by a monitor such as an LCD (Liquid Crystal Display) or a CRT (Cathode Ray Tube), and displays imaging-order information, the acquired image, and the like according to the display signal input from the controller of the console 4 or the display signal input from the imaging controller 2 via the console 4.

The display 41 also displays an image for display based on the processed image data.

The display 41 may be a remote display connected to the movable vehicle 100A by wire or wirelessly. This makes it possible for the user to check various kinds of information from a place away from the radiation emitting apparatus 3 of the movable vehicle 100A.

Also, a sub monitor other than the display 41 may be connected by wire or wirelessly.

The communication unit 42 includes a wired communication IF 42a in which an communication cable extending from the imaging analysis device is inserted for wired communication with the outside, or a wireless communication IF 42b for wireless communication with the outside. The communication unit 42 can switch the connection method to a wired one or a wireless one based on the control signal from the controller.

The power supply unit 5 includes a battery (internal power supply) 51, a power distributor 52, a power cable 53, and the like.

The battery 51 can supply the electric power stored in itself to the power distributor 52, or store the electric power supplied from the power distributor 52.

The power distributor 52 has a power cable 53 provided with a plug 53a at its tip, and electric power can be externally supplied by inserting the plug 53a into a nearby electrical outlet.

Then, the power distributor 52 distributes the electric power supplied from the battery 51 or outside to each part of the movable vehicle 100A.

A wiring for distributing electric power from the power distributor 52 to each part is omitted in FIG. 1, however, the power distributor 52 and each part are electrically connected with a wiring provided between them.

The power distributor 52 can be used for electric power of, for example, the voltage of 100 V or 200 V and the frequency of 50 Hz or 60 Hz. For this reason, electric power can be supplied from either a household power source or a commercial power source.

The above voltage and frequency are examples in using the radiation imaging system 100 in Japan. It is possible to be used in other countries or regions with the power distributor 52 differently designed.

### [Configuration of Radiation Imaging Apparatus]

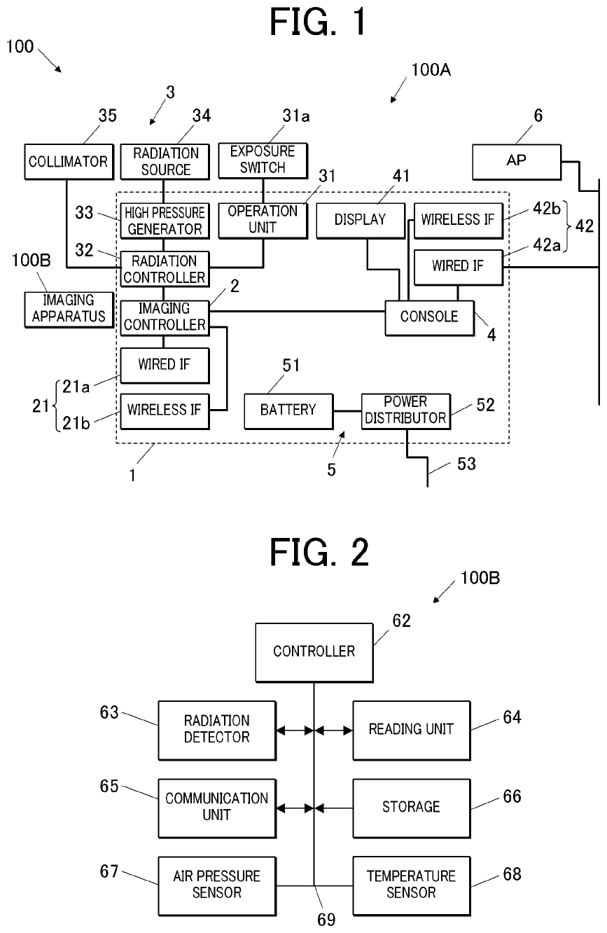

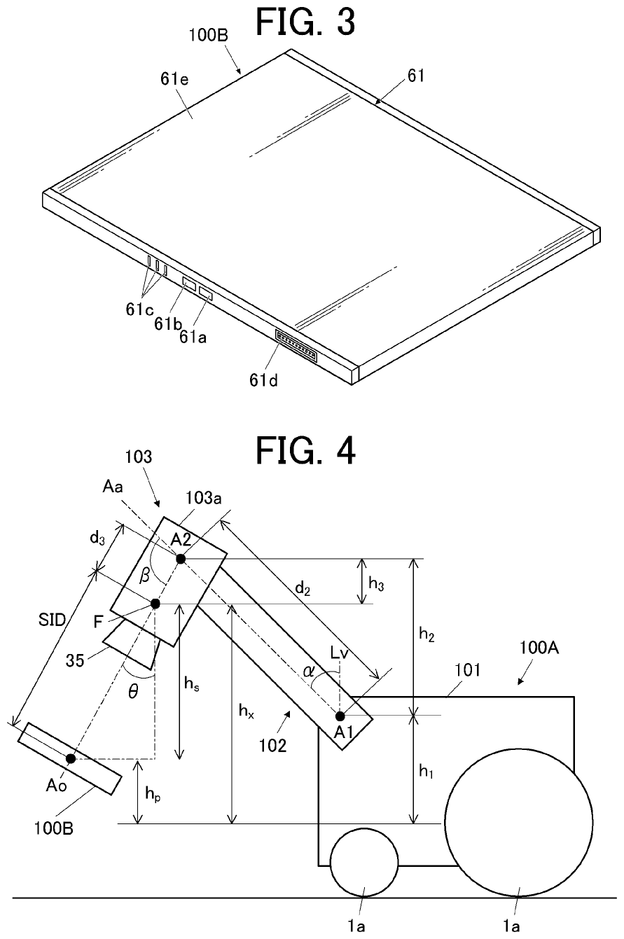

Next, a specific configuration of the imaging apparatus 100B included in the radiation imaging system 100 will be described. FIG. 2 is a block diagram showing the electrical configuration of the imaging apparatus 100B, and FIG. 3 is a perspective view of the imaging apparatus 100B.

In FIG. 3, an apparatus of a panel-shaped portable type is shown as the imaging apparatus 100B. However, the present invention is also applicable to an apparatus formed integrally with a support or the like, which may be called a stationary radiation imaging apparatus.

The imaging apparatus 100B according to the present embodiment is one of a so-called indirect type that converts an emitted radiation into an electromagnetic wave of another wavelength such as visible light to acquire an electric signal. The imaging apparatus 100B includes a housing 61 that accommodates, as shown in FIG. 2 and FIG. 3, a controller 62, a radiation detector 63, a reading unit 64, a communication unit 65, a storage 66, an air pressure sensor 67, a temperature sensor 68, and a bus 69 connecting the units 61 to 68.

The controller 62 includes a central processing unit (CPU), a random access memory (RAM), and the like. In response to control signals received from an external device such as console 4, the CPU of the controller 62 reads various programs stored in the storage 66 and loads them in the RAM, executes various processes according to the loaded programs, and integrally controls the operation of each part of the imaging apparatus 100B.

The radiation detector 63 includes a substrate in which pixels are arranged in a two-dimensional form (matrix) each provided with a radiation detection element that generates charges depending on the dose of the received radiation X, a switch element, and the like.

The radiation detector 63 may be a so-called indirect type radiation detector that incorporates a scintillator and the like, converts the emitted radiation X into light of a different wavelength (such as visible light) with the scintillator, and generates charges depending on the light after conversion. Alternatively, the radiation detector 63 may be a so-called direct type radiation detector that generates charges without a scintillator and the like.

The reading unit 64 can read the amount of charges released from each pixel as a signal value, and generate image data from a plurality of signal values.

The communication unit 65 can receive various kinds of control signals, data, and the like from an external device, and can send various kinds of control signals, generated image data, and the like to the external device.

The storage 66 is constituted by a non-volatile semiconductor memory, a hard disk or the like, and stores various programs executed by the controller 62, parameters necessary for execution of processes using the program, and the like.

The storage 66 can further store image data generated by the reading unit 64 and various kinds of data processed by the controller 62.

The air pressure sensor 67 and the temperature sensor 68 will be described later.

As shown in FIG. 3, the housing 61 is provided with a power switch 61a, an operation switch 61b, an indicator 61c, a connector 61d , and the like on its side surface.

One of the surfaces of the housing 61 is a radiation incident surface 61e.

The radiation incident surface is the surface of the housing 61 in the following description, however, it may be a surface of the substrate constituting the radiation detector 63 described above, or a surface of the scintillator.

The imaging apparatus 100B configured as described above stores electric charges corresponding to the dose of radiation in each pixel in response to receiving radiation while each switch element of radiation detector 63 is turned off by the controller 62. When the controller 62 turns on each switch element and charges are released from each pixel, the reading unit 64 converts each charge amount into a signal value and reads it as image data.

### [Configuration of Movable Portion of Movable Vehicle]

Next, the movable portion of the movable vehicle 100A will be described. FIG. 4 is a side view of the movable vehicle 100A and the imaging apparatus 100B.

As shown in FIG. 4, the movable vehicle 100A is configured to include a movable vehicle main body 101, an arm 102, and a radiation emitter 103.

In the present embodiment, the imaging controller 2, the console 4, and the power supply unit 5 described above are stored in the movable vehicle main body 101.

Further, in the present embodiment, the radiation emitter 103 has a case 103a, and the radiation source 34 of the radiation emitting apparatus 3 is stored in the case 103a, and the collimator 35 is attached to the end of the case 103a.

Further, a feeder (not shown in the drawing) for connecting the high pressure generator 33 and the radiation source 34 of the radiation emitting apparatus 3 passes through the arm 102.

A lower end of the arm 102 is pivotally supported by the movable vehicle main body 101 with the first rotation axis A1 horizontally extending in the movable vehicle main body 101 (for example, in the direction orthogonal to the sheet of FIG. 4), such that the arm 102 is rotatable. That is, it is possible to move the upper end of the arm 102 up and down.

An angle (hereinafter, arm rotation angle α) between the vertical line Lv and a straight line (hereinafter, arm axis Aa) along the extending direction of the arm 102 can be set to any value, as long as the middle or tip of the arm 102 does not touch the movable vehicle body 101 or the floor.

The position of the movable vehicle main body 101 where the lower end of the arm 102 is pivotally supported is not particularly limited. However, as shown in FIG. 4, it is preferable to pivotally support at the front end of the movable vehicle main body 101 from the viewpoint of widening the space for imaging under the radiation emitter 103.

Here, the arm rotation angle α is defined to be the angle between the vertical line Lv and the arm axis Aa, but may be defined on the basis of another line or plane (for example, a horizontal plane).

The case 103a of the radiation emitter 103 is rotatably supported at the tip of the arm 102 at the tip of the arm 102 by a second rotation axis A2 extending in parallel with the first rotation axis Al. That is, it is possible to change the direction of the radiation emission port (collimator 35).

The angle (hereinafter, emitter rotation angle β) between the arm axis Aa and the straight line (hereinafter, optical axis Ao of radiation) connecting the focal point F of the radiation emitted by the radiation source 34 and the center C of the opening formed by the shielding plate(s) in the collimator 35 can be set to any value as long as the case 103a and the collimator 35 do not contact the arm 102.

In the example of FIG. 4, the focal point F of the radiation is located between the second rotational axis A2 and the collimator 35. Alternatively, the focal point F may be located on an extension of the second rotational axis A2 or the second rotation axis A2 may be located between the focal point F and the collimator 35. On the contrary, the second rotation axis A2 may not be on the extension of the optical axis Ao of the radiation.

In the above, the emitter rotation angle β is defined by the angle formed by the arm axis Aa and the optical axis Ao of the radiation, but the emitter rotation angle may be defined based on another line or plane (for example, vertical line or horizontal plane).

In the vicinity of the first rotation axis A1, a first angle detector for detecting the angle of the arm rotation angle α is provided. In the vicinity of the second rotation axis A2, a second angle detector for detecting an angle of the emitter rotation angle β is provided.

Such an angle detector can be configured, for example, by a potentiometer using a variable resistor or a rotary encoder using a pulse counter.

The numerical values of the arm rotation angle α and emitter rotation angle β each detected by the angle detector are sent to the imaging controller 2 and the console 4 as needed, and are displayed on the display 41.

### [Radiation Emission Angle and Focal Height]

In the movable vehicle 100A described above, the distance from the first rotation axis A1 to the second rotation axis A2 is d₂, the distance from the second rotation axis A2 to the focal point F of the radiation is d₃, a predetermined reference height in the movable vehicle main body 101 (hereinafter referred to as an apparatus reference height) to the first rotation axis A1 is h₁. These values are known at the time of design and manufacture of the movable vehicle 100A.

Then, the angle (hereinafter, radiation emission angle θformed by the optical axis Ao of the radiation and the vertical line Lv is represented by the following equation (1), and the height from the apparatus reference height to the focal point F of the radiation (hereinafter, focal height hₓ) is represented by the following equation (2).

θ=180°−(α+β) (1)

hₓ=h₁+d₂ cos α−d₃ cos θ (2)

Here, the angle formed between the optical axis Ao of the radiation and the vertical line Lv is the radiation emission angle θ, but may be defined based on another specific (predetermined) plane or line.

In the above equation, “degree” is used as the unit of angle, but it may be converted to another units such as radian as appropriate to calculate the trigonometric function.

The imaging controller of movable vehicle 100A or the controller of console 4 executes a function of detecting the height of the radiation source by performing the above calculation based on the arm rotation angle α and the emitter rotation angle β detected by the first and second angle detectors.

FIG. 4 shows, as a conceptual diagram, an example in which rotation occurs with the straight line orthogonal to the sheet surface of FIG. 4 as the only rotation axis. However, the rotation direction need not be limited to the example shown in FIG. 4. A rotation mechanism which has a rotation axis in another direction (for example, the direction along the sheet of FIG. 4) for rotation may be used in combination. Even in such a case, the radiation emission angle θ and the focal height hₓ can be determined by calculation, for example, by acquiring the distance between rotation axes adjacent to each other, acquiring the angle between the planes orthogonal to the respective rotation axes, and adding the distance taking into consideration of the angle between the respective planes.

Further, FIG. 4 shows an example in which the second rotation axis A2 is on the arm axis Aa. However, the present invention is not limited to such a configuration, but may be configured such that, for example, one or more rotation axes other than the second rotation axis A2 may be on the arm axis Aa or at positions separate from the arm axis Aa. Even in such a configuration, it is possible to grasp the distance (the amount of separation) between the arm axis Aa and the other rotation axis in the step of design or manufacture. Therefore, the θ and hₓ can be calculated using these values.

In addition to the rotation mechanism, it is also possible to combine one or more expansion mechanisms or lifting mechanisms. Even in such a case, the radiation emission angle θ and the focal height hₓ can be determined by calculation, for example, by considering the increase or decrease of the distance due to the extension mechanism or the lifting mechanism in the distance between the rotation axes.

### [Placed Height of Imaging Apparatus]

The imaging apparatus 100B includes, as shown in FIG. 2, an air pressure sensor 67 that measures the atmospheric pressure of the height where the imaging apparatus 100B itself is located.

The air pressure sensor 67 may be built in the imaging apparatus 100B or may be provided outside the housing.

From the viewpoint of avoiding reflection in the image, the air pressure sensor 67 is preferably arranged on the periphery of the substrate constituting the radiation detector 63 or on the back side of the substrate.

Also, as shown in FIG. 2, the imaging apparatus 100B is equipped with a temperature sensor 68 for measuring the temperature around the imaging apparatus 100B.

The attached position of the temperature sensor 68 is not particularly limited, but is preferably arranged away from the heat emitting point(s) in the imaging apparatus 100B.

The relationship between the air pressure P measured by the air pressure sensor 67, the temperature (air temperature) T measured by the temperature sensor 68, and the placed height h of the imaging apparatus 100B is represented by, for example, the following equation (3).

h={((₀/P)^{(1/2 257)}−1)×(T+273.15)}/0.0065 (3)

Here, P₀ is the reference air pressure at a reference height such as the sea level.

The imaging controller of movable vehicle 100A or the controller of console 4 executes a function of detecting the height of the imaging apparatus 100B by performing the above calculation based on the measurement value (air pressure P) by the air pressure sensor 67 and the measurement value (temperature T) by the temperature sensor 68.

The placed height h may be calculated in the imaging apparatus 100B.

The reference air pressure Po changes depending on the weather change. Therefore, it is necessary to correct the measured air pressure depending on the weather conditions at the time of measurement.

For example, a specific measurement value may be measured by the air pressure sensor 67 when the imaging apparatus 100B is arranged at a specific position, stored, and used for correction of the detected height of the imaging apparatus 100B. More specifically, a calibration unit on which the imaging apparatus 100B can be mounted is provided at a specific height (for example, the movable vehicle body 101) in the movable vehicle 100A. The measurement values by the air pressure sensor 67 and the temperature sensor 68 when the imaging apparatus 100B is stored in the calibration unit are stored in the storage 66 as specific measurement values.

Thus, the height of the movable vehicle imaging apparatus 100B can be calculated as a relative height with respect to the calibration unit as a reference, based on the air pressure and temperature measured at that time, and the air pressure and temperature measured when placed on the calibration unit.

Here, when the height of the calibration unit is hₜ and the air pressure in the calibration unit is Pₜ, hₜ is represented by the following equation (4).

Hₜ={((P₀/Pₜ)^{1/5 257)}−1)×(T+273.15)}/0.0065 (4)

Further, from the above equation (4), the reference air pressure Po is represented by the following equation (5).

P₀=Pₜ[{0.0065hₜ/(T+273.15)}+1]^{5.257} (5)

Here, if hₜ=0 in order to calculate the height at which calibration is performed, P₀=Pₜ.

Therefore, the relative height h_{diff} from the height at which calibration is performed is represented by the following formula (6), using the air pressure Pₜ measured at the height where calibration is performed and the air pressure P measured at any height.

h_{diff}={((Pₜ/P)^{(1/5 257)}−1)×(T+273.15)}/0.0065 (6)

For example, when the apparatus reference height is set to the height where calibration is performed, h_{diff} is hₚ.

In the above-described example, the height hₚ of the imaging apparatus 100B is acquired from the measurement value of one air pressure sensor 67. However, the height hₚ can also be acquired from the measurement values of respective air pressure sensors 67 which are included in the imaging apparatus 100B. Specifically, a portion provided with the air pressure sensor 67 is referred to as a first portion Pi, and at a second portion P₂ which is (at a position) different from the first portion P₁ are a second air pressure sensor 67A for measuring the atmospheric pressure at the height where the imaging apparatus 100B is located and a second temperature sensor 68A. As a result, functions to detect the height of the first portion P₁ by performing the above-mentioned calculation and to detect the height of the second portion P₂ based on the measurement value measured by the air pressure sensor 67 are provided. Then, by calculating the average value of each of the height of the first portion P₁ and the height of the second portion P₂ or by using an arithmetic method such as weighted averaging that takes the arrangement of each air pressure sensor 67 into account, the height of another portion in the imaging apparatus 100B and different from the first portion P₁ and the second positon P₂ may be calculated and used as the height of the imaging apparatus 100B.

Alternatively, the imaging apparatus 100B may be provided with a gravity sensor for correcting the calculated value for the imaging apparatus 100B from the air pressure sensor 67.

The difference in height h shown in FIG. 4, which is between the focal point F of the radiation and the imaging apparatus 100B is represented by the following formula (7) using the focal height hₓ and the placed height hₚ of the imaging apparatus 100B.

Hₛ=hₓ−hₚ (7)

Then, SID, which is the distance from the focal point F of the radiation to the radiation incident surface of the imaging apparatus 100B, is represented by the following formula (8) using the height difference hₛ calculated here and the radiation emission angle θ represented.

SID=hₛ/cosθ (8)

The imaging controller of the movable vehicle 100A or the controller of the console 4 executes a function of calculating the distance from the focal point F of the radiation generated by the radiation source 34 to the imaging apparatus 100B on the basis of the height of the detected radiation source and the height of the imaging apparatus 100B.

Further, the console 4 is configured to cause the display 41 to display the calculated distance.

### [Method of Calculating Arrangement Angle of Imaging Apparatus]

Next, the method of calculating the angle of the radiation incident surface of the imaging apparatus 100B will be described.

When there are two different portions P₁ and P₂ located along the radiation incident surface in the imaging apparatus 100B, the distance between them is set to be dₚ, and the height difference between the first portion P₁ and the second portion P₂ is set to be hₚ₂. The angle formed by the radiation incident surface of the imaging apparatus 100B and the horizontal surface (hereinafter referred to as an incident surface inclination angle %) is represented by the following formula (9).

θₚ=sin⁻¹(hₚ₂/dₚ) (9)

Here, the angle formed between the radiation incident surface and the horizontal plane is defined as the radiation irradiation angle θ, but may be defined based on another specific plane or line.

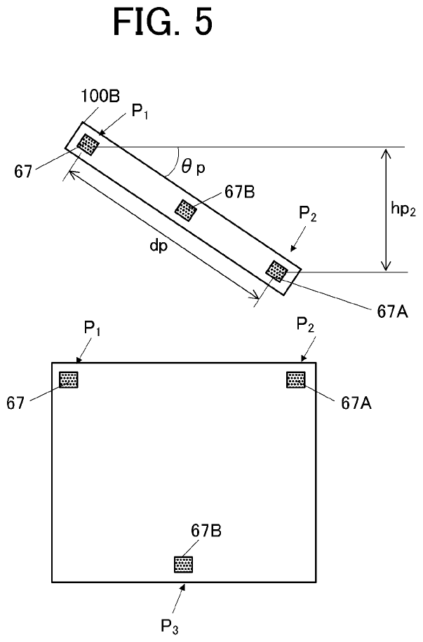

In order to calculate the incident surface inclination angle θₚ, as shown in FIG. 5, for example, the air pressure sensor 67 is provided at the first portion Pi, and a temperature sensor 68 is provided at a second portion P₂ which is different from the first portion P₁, along with a second air pressure sensor 67A which measures the atmospheric pressure at the height of itself. It executes a function of detecting the height of the first portion P₁ by the calculation as described above, and detecting the height of the second portion P₂ based on the measurement value measured by the air pressure sensor 67.

The imaging controller of the movable vehicle 100A or the controller of the console 4 further executes a function of calculating the incident surface inclination angle % based on the detected height of the first portion P₁ and the height of the second portion P₂.

Two temperature sensors 68 may be arranged as well as the air pressure sensors 67 and 67A, but it may be one temperature measuring device. The temperature of the imaging apparatus 100B may be different from that of surrounding air due to operation or heat transfer from the subject S. Therefore, the temperature measured by the temperature sensor arranged at the other part of the imaging apparatus 100B may be used as the temperature of the surrounding air.

In some imaging procedures, it is desirable that radiation is emitted to the imaging apparatus 100B such that the optical axis Ao of the radiation is orthogonal to the radiation incident surface. In other imaging procedures, it may be desirable that radiation is emitted to the imaging apparatus 100B such that the optical axis Ao of the radiation is inclined to the radiation incident surface by a particular angle.

An angle formed by the optical axis Ao of the radiation and the radiation incident surface of the imaging apparatus 100B (hereinafter, the imaging apparatus arrangement angle θ_{diff}) is represented by the following formula (10) using the radiation emission angle θ calculated by the above method and the incident surface inclination angle θₚ calculated here.

θ_{diff}=θ−θₚ (10)

The imaging controller of the movable vehicle 100A or the controller 4 of the console 4 executes a function of calculating and outputting the difference (imaging apparatus arrangement angle θ_{diff}) between the radiation emission angle θ and the incident surface inclination angle θₚ. Specific output methods include displaying on display, transmitting the calculated value to an external display device (not shown), and the like.

By displaying the imaging apparatus arrangement angle θ_{diff} in this manner, the user can adjust the radiation emission angle θ and the incident surface inclination angle θₚ while checking the current imaging apparatus arrangement angle θ_{diff}. As a result, the imaging apparatus placement angle θ diff can be easily made a desired angle.

In the above-described example, one angle is calculated with two air pressure sensors 67. Instead, a third air pressure sensor 67B may be provided at a non-linear third portion P₃ which is not on a straight line connecting the two air pressure sensors 67.

In this way, the imaging apparatus arrangement angle θ_{diff} can be determined three-dimensionally, and the imaging apparatus arrangement angle θ_{diff} of the imaging apparatus 100B with respect to the optical axis of the radiation can be unambiguously determined.

In the radiation imaging system 100 according to the present embodiment, as described above, the SID and the imaging apparatus arrangement angle θ_{diff} formed by the radiation optical axis Ao and the radiation incident surface are calculated, and the calculation results are displayed on the display.

Accordingly, the user can easily know the center of gravity of the imaging apparatus 100B and the height of the center.

### [Basic Flow of Imaging]

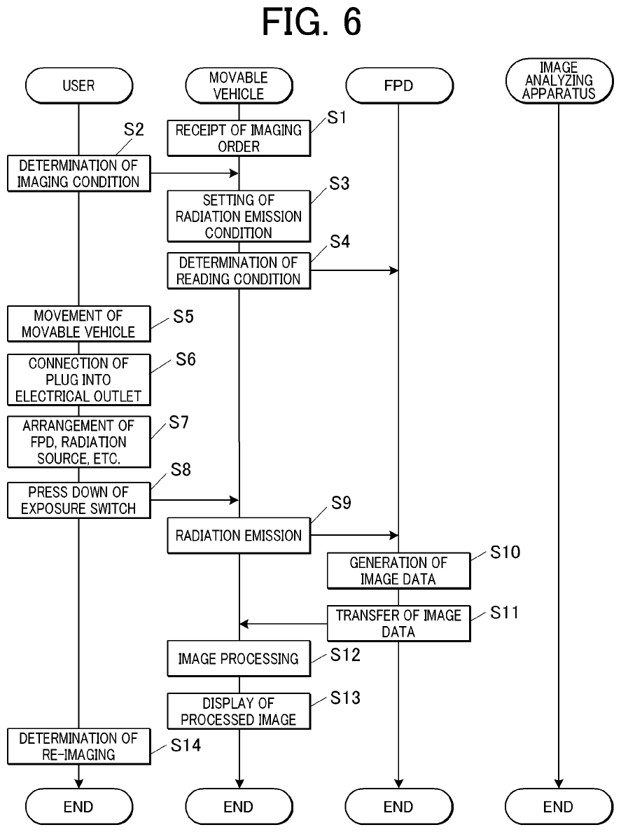

Next, a basic flow of imaging using the radiation imaging system 100 will be described. FIG. 6 is a ladder chart showing a basic flow of inspection using the radiation imaging system 100 of the present embodiment.

In the first imaging preparation operation, the console 4 receives an imaging order from the RIS or the like via the access point 6 or the like (step S1).

Then, the user determines various imaging conditions based on the received imaging order (step S2). Specifically, the user operates the operation unit 31 to select from any of the imaging conditions or to input numerical values. When serial imaging is performed, the frame rate, imaging time, number of frames, etc. are also determined.

When the imaging condition is determined, the imaging controller 2 of the movable vehicle 100A sets, on the basis of the input content via the operation unit 31, the radiation emission conditions of the high-pressure generator 33, the imaging range of the collimator 35, the kind of filter, etc. are set according to a command from the console 4 (step S3), and the read conditions (binning range etc.) of the imaging apparatus 100B are also set (step S4).

The various imaging conditions may not be determined by the user, but may be automatically set by the console 4.

If multiple imaging apparatuses 100B are provided in the radiation imaging system 100, one of them is selected here.

After the imaging preparation is complete, the user starts positioning operation.

In the positioning operation, first, the movable vehicle 100A is moved to the vicinity of the subject S (step S5). Then, the plug 53a of the power cable 53 is inserted into the outlet so that electric power can be supplied from the outside (step S6). As mentioned above, the power supply unit 5 of the movable vehicle 100A is compatible with both household power source and commercial power source. Therefore, electric power can be supplied even at the house of the subject S, not to mention at an operating room, intensive care unit, sick room, and the like.

Then, the imaging apparatus 100B, the radiation source 34, and the subject S are arranged at the positions suitable for imaging (step S7). The radiation source 34 is arranged so as to face the imaging apparatus 100B with the subject S therebetween by inserting the imaging apparatus 100B between the bed and the examination target portion of the subject S lying thereon, bringing the imaging apparatus 100B into contact with the subject S, and the like.

Specifically, the user adjusts the position of the radiation emitter 103 and the orientation of the emission port while observing the SID displayed on the display 41 and the imaging apparatus arrangement angle θ_{diff}.

At this time, by turning on a light source (not shown) provided in the case 103a of the radiation emitter 103, for example, it is possible to illuminate the same range as the emission field to be illuminated by the radiation with visible light. In this way, the position of the imaging apparatus 100B can be easily adjusted by aligning the optical axis of visible light with the center of the radiation incident surface.

After the positioning operation, the user performs imaging operation.

In the imaging operation, the user presses the exposure switch 31a (step S8). Then, the imaging controller 2 adjusts the timing of the high-voltage generator 33 and the imaging apparatus 100B and executes imaging Specifically, when the exposure switch 31a is pressed for the first step, preparation of the radiation source 34 (rotation of the rotor when it is a rotary anode type) is performed, and then the imaging apparatus 100B is ready for imaging.

Here, the user confirms whether or not the radiation emitting apparatus 3 and the imaging apparatus 100B are in a state ready for imaging. Here, in the case where the movable vehicle 100A is provided with a state display for displaying whether or not the radiation emitting apparatus 3 and the imaging apparatus 100B are in a state ready for imaging, the user confirms it according to the display content of the state display. With such a configuration, the user can confirm whether or not it is ready for imaging at a glance. Then, it is possible to confirm the state without checking the display where various kinds of other information are displayed, such as the display 41 of the console 4.

If it is ready for imaging according to the confirmation, the user presses the exposure switch 31a for the second step. Then, the radiation controller 32 controls the high pressure generator 33 to generate radiation during the preset time continuously or in a pulse of a preset period (step S9) and the imaging controller 2 repeats reading and storing at the frame rate set for the imaging apparatus 100B (generates image data, step S10).

When a preset imaging time has elapsed, the imaging controller 2 stops emitting radiation and reading by the imaging apparatus 100B. When the exposure switch 31a is released during imaging, the radiation exposure and the reading of the imaging apparatus 100B are also stopped.

After the user has finished imaging, the radiation imaging system 100 starts operation for confirmation of the image.

First, the imaging apparatus 100B transfers the generated dynamic image data to the console 4 via the communication unit 21 of the movable vehicle 100A (step S11). Then, the console 4 sequentially performs image processing on image data of a plurality of frames constituting the transferred dynamic image data to generate processed dynamic image data (step S12).

Then, the console 4 displays a dynamic image based on the processed dynamic image data on the display 41 (step S13). In order to quickly display an image, a dynamic image on which simple image processing has been performed may be displayed during imaging.

After imaging is completed and image processing is performed on image data of all frames, the dynamic image can be checked on the display 41.

The user checks the dynamic image displayed on the display 41 and determines whether or not re-imaging is necessary (step S14).

If the user determines that re-imaging is unnecessary (imaging is successful) as a result of the image check, the image data is stored in the console 4, transferred to an external device as needed, and the like.

Thus, a series of imaging is terminated.

According to the radiation imaging system 100 according to the first embodiment described above, the SID can be easily adjusted as the position of the radiation source or the imaging apparatus 100B can be adjusted while viewing the current SID displayed, such that the displayed SID is the desired value.

### Second Embodiment A

Next, a second embodiment A will be described with reference to the drawings. Here, the same components as in the first embodiment are denoted by the same reference numerals, and the description thereof is omitted.

### [Radiation Imaging System]

First, an outline of the radiation imaging system 200 according to the present embodiment will be described. FIG. 7 is a block diagram of a radiation imaging system 200 of the present embodiment.

As shown in FIG. 7, the radiation imaging system 200 according to the present embodiment is configured to include a system main body 100A and an imaging apparatus(es) 100B as in the radiation imaging system 100 according to the first embodiment, or is configured to include the imaging apparatus 100B, the system main body 100A of the first embodiment from which the measurement/display function of the SID is removed, and a body motion detecting apparatus 100C.

The body motion detecting apparatus 100C is communicably connected to the system main body 100A. FIG. 7 exemplifies a case where the body motion detecting apparatus 100C is connected by wire, but may be connected wirelessly.

The body motion detecting apparatus 100C can detect the motion of the subject S during imaging

The console 4 may also function as the body motion detecting apparatus 100C, instead of the body motion detecting apparatus 100C as an independent apparatus.

In the radiation imaging system 200 according to the present embodiment configured in this way, as well as the radiation imaging system 100 according to the first embodiment, it is possible to take at least one of a still image and serial images of the subject S by irradiating the subject S in front of the imaging apparatus 100B with radiation emitted from the system main body 100A.

That is, in response to one imaging operation (depression of the exposure switch 31a, a dynamic image can be acquired by repeated imaging of the subject S (the imaging apparatus 100B repeats charge accumulation and signal value reading several times in a short time).

[Body motion Detecting Apparatus]

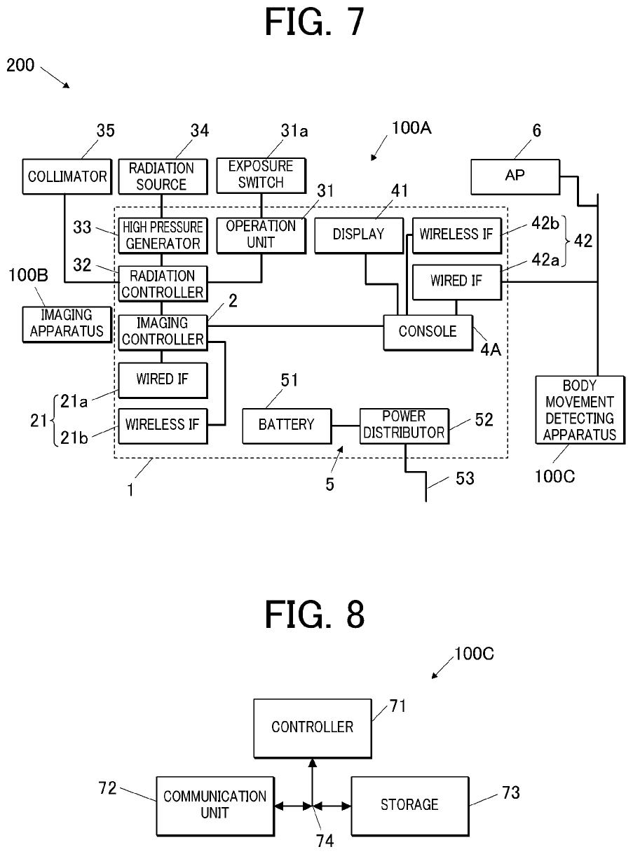

Next, a specific configuration of the body motion detecting apparatus 100C included in the radiation imaging system 200 will be described. FIG. 8 is a block diagram showing the configuration of the body motion detecting apparatus 100C.

As shown in FIG. 8, the body motion detecting apparatus 100C includes a controller 71, a communication unit 72, a storage 73, and a bus 74 connecting the respective units 71 to 73.

The controller 71 includes a central processing unit (CPU), a random access memory (RAM), and the like. In response to control signals received from an external device such as the radiation emitting apparatus 3 or console 4, the CPU of the controller 71 reads various programs stored in the storage 73 and loads them in the RAM, executes various processes according to the loaded programs, and integrally controls the operation of each part of the imaging apparatus 100B.

The communication unit 72 can receive various control signals from the system main body 100A, receive image data from the imaging apparatus 100B, send various processing results (judgement results with body motion described later) to the system main body 100A, and the like.

The storage 73 is constituted by a non-volatile semiconductor memory, a hard disk or the like, and stores various programs executed by the controller, parameters necessary for execution of processes using the program, and the like.

The storage 73 can further store image data received from the imaging apparatus 100B.

The controller 71 of the body motion detecting apparatus 100C configured as described above functions as follows.

For example, the controller executes a function of acquiring image data of multiple images from the imaging apparatus 100B.

In this embodiment, the controller acquires the first image data (not necessarily the data of the first image) from the imaging apparatus 100B in synchronization with the timing when the radiation imaging is started. Thereafter, images are acquired by imaging repeatedly performed at predetermined time intervals. Image data may be acquired each time the imaging apparatus 100B generates image data, or may be acquired once for a predetermined number of times of image data generation.

The acquired image data is stored in the storage.

In addition, in the acquired image data of each image, the controller 71 executes a function of specifying specific portions P₄ to P₇ which are required to cause no body motion other than the specific body motion to be diagnosed.

The “specific body motion to be diagnosed” includes, for example, beating of the heart, expansion and contraction of the lungs, vertical motion of the diaphragm, motion of surrounding bones associated with the above motion, blood flow, and the like.

On the other hand, the “body motion other than the specific body motion to be diagnosed” includes displacement, rotation, and the like of the entire region to be imaged.

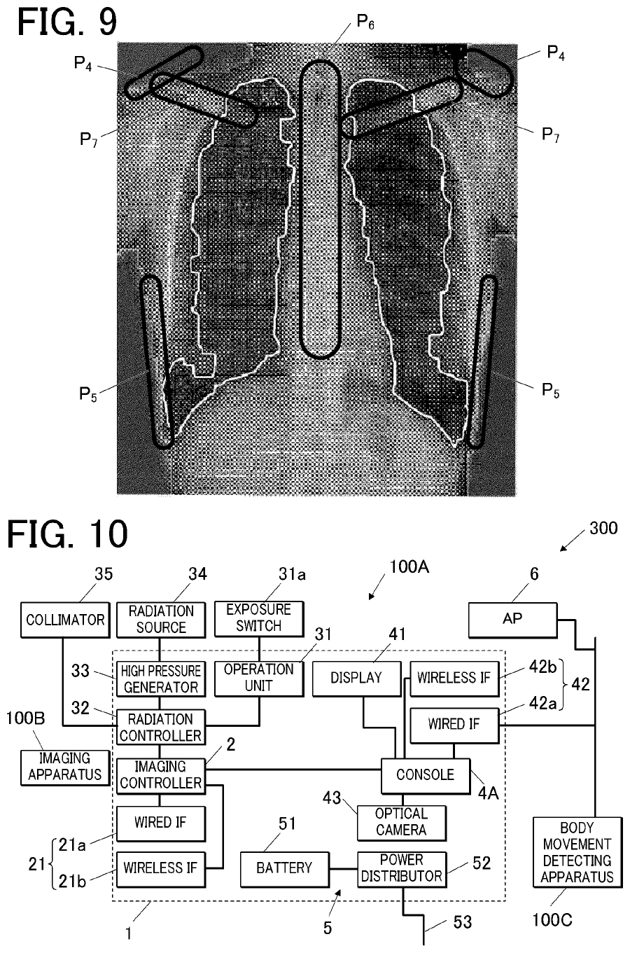

For this reason, the “specific portions P₄, P₅” may be a shoulder(s) or flank(s) as shown in FIG. 9, for example. Further, in the present embodiment, since a radiographic image is used, bone extraction may be performed to set the spine and clavicle as the specific portions P₆ and P₇.

Further, the controller 71 executes a function of detecting motion of the specified specific portions P₄ to P₇ on the basis of the image data on multiple images.

Specifically, the controller 71 compares the specific portions commonly appearsing in two or more images among the acquired multiple images, and measures the displacement amount of each of the specific portions (or a small region or point in the respective specific portion) as the moving amount of each of the specific portions.

It is also possible to separately detect the moving amount of each of the specific portion P₄ to P₇ into a first direction component that is a direction in which a specific body motion is performed and a second direction component which is different from the first direction.

If each of the specific portion P₄ to P₇ is a portion where it is difficult to detect the motion, an indicator (marker M) may be attached to a region overlapping the specific region of the body surface of the subject S, so that the motion may be detected on the basis of the displacement amount of the indicator attached to the subject S.

The controller 71 also executes a function of judging the presence or absence of a body motion different from the specific body motion on the basis of the detected motion.

Specifically, the controller 71 judges that there is a body motion different from the specific body motion when the moving amount of each of the specific portion, which is the degree of the detected motion, exceeds a predetermined threshold, and that there is no body motion (body motion does not affect imaging) when the moving amount is the threshold or less.

When the moving amount of the specific portion is detected separately in the first direction component and the second direction component, a first threshold for comparing with the moving amount in the first direction and a second threshold for comparing with the moving amount in the second direction is preferably set.

For example, in serial imaging of the lung field, certain vertical body motion associated with breathing occur to some extent on the shoulder, but body motion does not generally occur in the lateral direction. Therefore, if body motion in the lateral direction is detected in serial imaging of the lung field where the shoulder is the specific portion, it can be determined that the subject S has lost balance during imaging

In addition, when the vertical body motion on the shoulder is too large, it may also be determined that a vertical body motion different from the specific body motion is occurring on the shoulder.

The spine is not usually displaced in any direction during serial imaging. Therefore, if serial imaging is performed with the spine as the specific portion, it is determined that any body motion detected in any direction is different from the specific body motion.

When the controller 71 judges that there is a body motion, the controller 71 may warn the user that body motion which may affecting the diagnosis has occurred.

Specifically, the controller 71 warns the user by displaying the warning on the display, making a speaker play sound, or turning on a lamp.

In addition, the controller 71 may interrupt the radiation emission from the radiation source in judging that there is body motion.

Specifically, if it is judged that there is body motion, the controller 71 sends a predetermined control signal to the console 4 or the imaging controller, and transmits a signal to stop the radiation emission to the radiation controller when the console 4 or the imaging controller receives the control signal.

The controller 71 may perform only one or both of the warning and the radiation emmision interruption.

[Basic flow of Imaging]

The flow of imaging using the radiation imaging system 200 according to the present embodiment is also basically the same as that using the radiation imaging system 100 according to the first embodiment (see FIG. 6).

In the arrangement of the imaging apparatus 100B and the radiation source 34 (step S7), the imaging apparatus 100B and the radiation source 34 can be arranged using the display function of the SID and the imaging apparatus arrangement angle θ_{diff} described in the first embodiment.

When the user presses the exposure switch 31a for the second step and radiation emission (step S10) and the generation of image data (step S11) are repeated, the body motion detecting apparatus 100C acquires image data and repeatedly determines whether or not there is body motion.

When a predetermined imagine time has elapsed without judging that there is a body motion by the body motion detecting apparatus 100C, imaging is terminated.

On the other hand, if the body motion detecting apparatus 100C judges that there is body motion during the imaging, the imaging is interrupted immediately after the determination.

### Second Embodiment B

Next, a second embodiment B will be described with reference to the drawings. Here, the same components as in the first embodiment are denoted by the same reference numerals, and the description thereof is omitted.

### [Radiation Imaging System]

First, an outline of the radiation imaging system 300 according to the present embodiment will be described. FIG. 10 is a block diagram of a radiation imaging system 300 of the present embodiment.

While the radiation imaging system 100 according to the above first embodiment detects body motion on the basis of image data generated by the imaging apparatus 100B, the radiation imaging system 300 according to the present embodiment detects body motion on the basis of image data generated by an optical camera 43.

Therefore, the radiation imaging system 300 according to the present embodiment includes an optical camera 43 in addition to the configuration described in the first embodiment.

As in the first embodiment, the body motion detecting apparatus 100C may not be an independent device, but the console 4 may also function as the body motion detecting apparatus 100C.

The optical camera 43 is communicably connected to the apparatus body (console 4) by wire or wirelessly. Then, image data of the taken image(s) (which may be a still image or dynamic images) is transmitted to the apparatus main body. The optical camera 43 also repeatedly performs imaging (including serial imaging) of the subject S during radiation imaging.

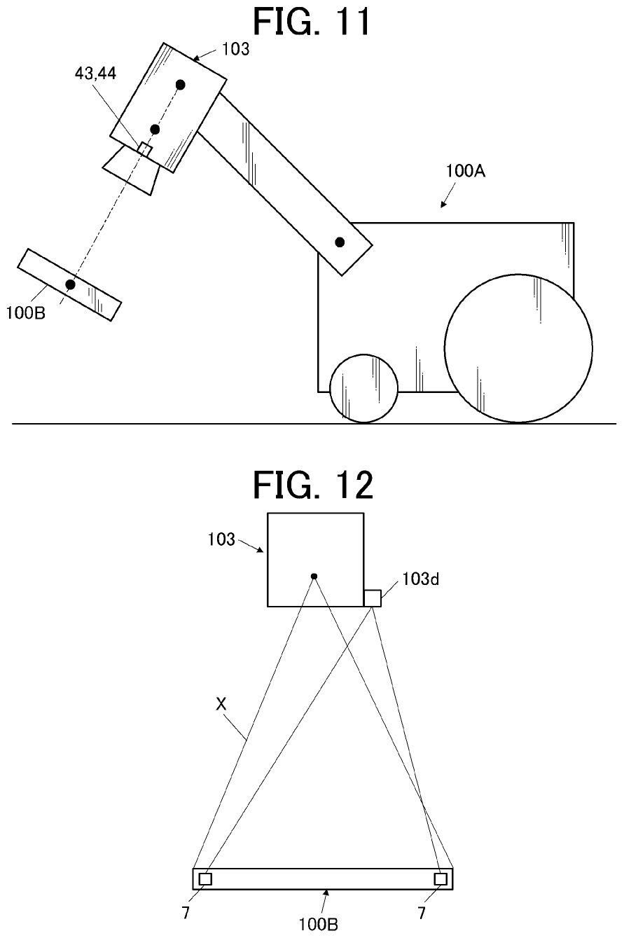

The position of the optical camera 43 is not particularly limited as long as imaging of the subject S is possible during radiation imaging, but is preferably provided on the radiation emitter 103 as shown in FIG. 11, for example.

The controller of the body motion detecting apparatus 100C according to the present embodiment executes a function of acquiring image data of multiple images from the optical camera 43.

The acquired image data may be stored in the storage, or may be discarded without being stored after usage for body motion detection.

The optical camera 43 cannot capture a specific part (spine or bone) inside the body. Therefore, in serial imaging using radiation imaging system 300 according to the present embodiment, the motion is preferably detected on the basis of the motion (displacement amount, displacement direction) of the indicator attached to the body surface of the subject S.

The indicator may be specified automatically, for example, by the body motion detecting apparatus 100C which identifies the color of the indicator, or may be specified on the basis of a region specified by the user according to the image displayed on the display acquired from the optical camera 43.

The indicator may be made of any material, be formed in any shape, and have any size as long as it can be identified by the body motion detecting apparatus 100C from the image data. For example, if the indicator is formed of a material having high radiation transmittance, it is possible to prevent the indicator from appearing in the radiographic image while determination is made whether or not there is body motion.

According to the body motion detecting apparatus 100C of the radiation imaging systems 200, 300 according to the second embodiments A and B described above, presence or absence of body motion is not judged for portion(s) other than the specific portion (the portion which does not affect imaging even if body motion occurs there). Only when body motion different from the specific body motion occurs at the specific portion(s), it is judged that there is body motion.

### EXAMPLES

Next, problems that may newly occur when the first and second embodiments are implemented, and specific examples for solving the problems will be described.

The techniques listed in the following examples may be used for a radiation imaging system other than the first and second embodiments.

### [Measurement Method (1)]

As described above, according to the conventional radiation imaging system, there is a problem that it is difficult to grasp the SID and adjust it to a predetermined SID because SID changes depending on the imaging procedure, imaging conditions, and status of the subject S.

In view of such a problem, according to the first embodiment, the problem is solved by displaying the SID calculated from the length and the angle of each part of the system. Alternatively, a stereo camera 44 capable of measuring the distance between itself and the subject S may be provided at the radiation emitter 103, at a portion from which relative position to the radiation emitter 103 does not change, or at a portion from which the relative position can be detected. The specific attachment place of the stereo camera 44 is preferably at the radiation emitter 103 as shown in FIG. 11.

The distance between the focal point F of the radiation and the stereo camera 44 is fixed at the time of design and production of the system main body 100A. Therefore, the SID can be calculated by adding or subtracting this distance to the distance measured by the stereo camera 44 and by further adding the estimated body thickness of the subject S.

If the SID calculated as described above is displayed on the display, as in the first embodiment, the SID can be easily adjusted as the position of the radiation source or the imaging apparatus 100B can be adjusted while viewing the current SID displayed, such that the displayed SID is the desired value.

### [Measurement Method (2)]

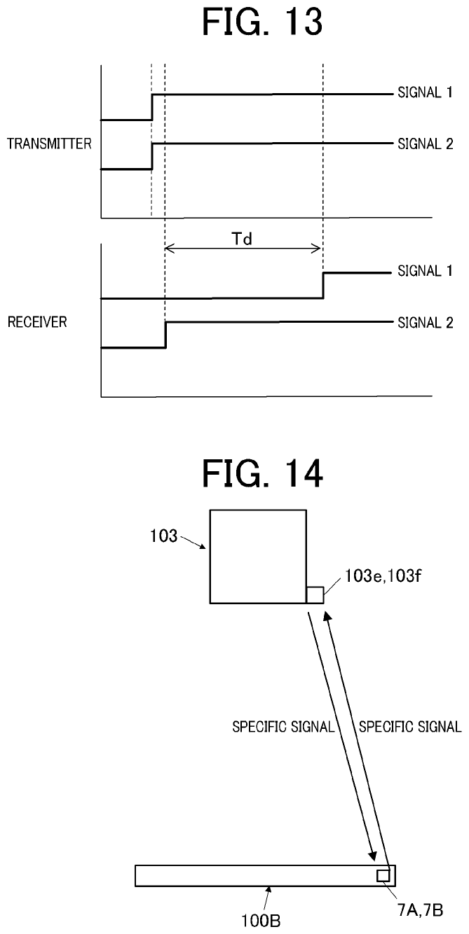

In view of the problem that it is difficult to grasp the SID and adjust it to a predetermined SID according the conventional radiation imaging system, a transmission unit 103d which simultaneously transmits a first signal and a second signal respectively traveling at a predetermined speed and at a speed different from the predetermined speed may be provided at the radiation emitter 103, at a portion from which relative position to the radiation emitter 103 does not change, or at a portion from which the relative position can be detected as shown in FIG. 12. Also, a receiver 7 is provided in the imaging apparatus 100B and receives the first and second signals. The console 4 or the like may execute a function of calculating the distance between the radiation emitter 103 and the imaging apparatus 100B on the basis of the difference Td (see FIG. 13) between the time when the receiving unit 7 receives the first signal and the time when the receiving unit 7 receives the second signal.

Signals such as sound waves and radio waves with different speeds can be used as the first and second signals, for example.

In this way, the current SID can be grasped, and imaging can be performed with an appropriate SID.

### [Measurement Method (3)]

In view of the problem that it is difficult to grasp the SID and adjust it to a predetermined SID according the conventional radiation imaging system, a first transmission unit 103e which transmits a specific signal and a first receiver 103f which receives the specific signal may be provided at the radiation emitter 103, at a portion from which the position relative to the radiation emitter 103 does not change, or at a portion from which the relative position can be detected as shown in FIG. 14. Also, in the imaging apparatus 100B, there are provided a second receiver 7A which receives a specific signal, and a second transmission unit 7B which transmits a specific signal immediately after the second receiver 7A receives the specific signal from the first transmission unit 103e or after a certain time has elapsed. There is also provided a controller which calculates the distance between radiation emitter 103 and imaging apparatus 100B based on the difference between the time when the first transmission unit 103e transmits the specific signal and the time when the first receiver 103f receives the specific signal from the second transmission unit 7B.

Signals such as sound waves and radio waves can be used as the specific signal, for example.

In this way, the current SID can be grasped, and imaging can be performed with an appropriate SID.

The respective multiple receivers 7 in the above description of the measurement method (2) may be arranged at three or more different reception points in the imaging apparatus 100B. In this way, the imaging apparatus arrangement angle θ_{diff} of the imaging apparatus 100B can be calculated.

Further, in this case, radio waves or sound waves of different frequencies may be transmitted from the transmission unit 103d to the respective receivers 7. In this way, interference of radio waves or sound waves can be prevented, and SID or the like can be more reliably calculated.

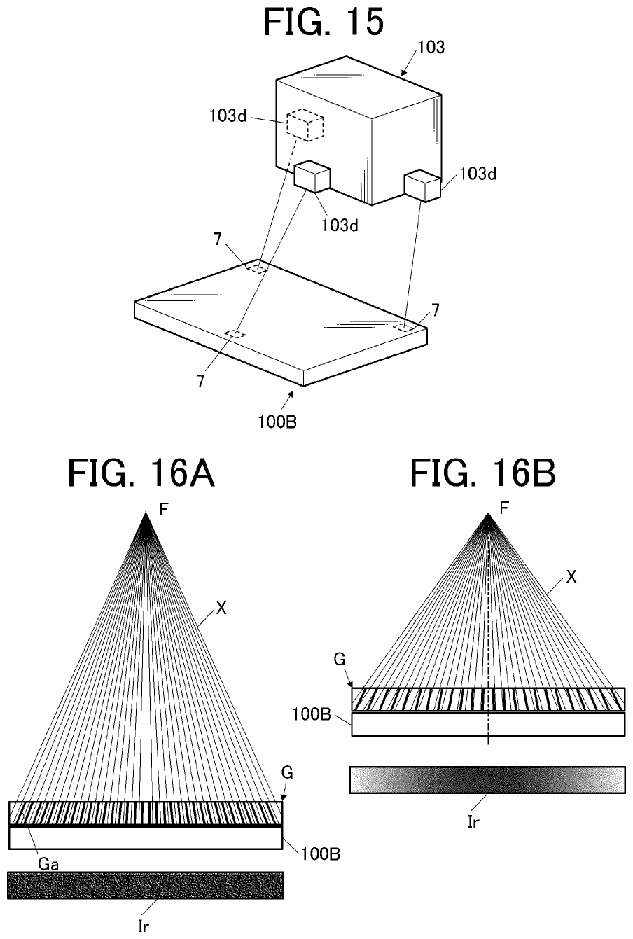

Further, in this case, as shown in FIG. 15, for example, the multiple transmission units 103d corresponding to the respective receivers 7 may be arranged in different places in the radiation emitter 103. In this way, interference can be more reliably prevented.

Also, the transmission units 103d may be arranged collectively, that is, in one place. As a result, the number of parts can be reduced, and the configuration including the imaging controller 2 and the radiation controller 32 can be simplified.

Also, instead of transmitting the first and second signals from the radiation emitter 103 to the imaging apparatus 100B, a transmission unit may be provided on the imaging apparatus 100B, and a receiver may be provided on the radiation emitter 103 side, such that the first and second signals may be transmitted from the imaging apparatus 100B to the radiation emitter 103. According to the configuration of this example, the imaging apparatus 100B may be difficult to be carried because of the controller which is likely to be heavy or large as many processes are performed in the receiver. However, in this way, it is possible to prevent the imaging apparatus 100B from becoming heavy and large.

In addition, an air pressure sensor 67 or a temperature sensor 68 may be provided, and based on the values measured by them, the time when the receiver receives the signal or the calculated distance may be corrected. In this way, distances and angles can be calculated more accurately.

Also, the frequency of the used sound waves may be outside the audible range. In this way, the user and the subject S do not feel uncomfortable with the sound in installation of the imaging apparatus 100B.

### [Measurement Method (4)]

In view of the problem that it is difficult to grasp the SID and adjust it to a predetermined SID according the conventional radiation imaging system, as shown in FIG. 16A, for example, the imaging apparatus 100B may be separated from the focal point F of the radiation by a predetermined distance, and a grid (radiation selective transmission part) G may be provided closer to the focal point F of the radiation than the imaging apparatus 100B. The grid G includes inclined thin plates Ga parallel to the emitted radiation when the optical axis Ao of the radiation is arranged to be orthogonal to the radiation incident surface at its center. In this way, the SID and the like may be calculated based on the radiographic image taken through the grid G.

As shown in FIG. 16A, when the focal point F of the radiation is separated from the imaging apparatus 100B by a predetermined distance and all the thin plates Ga forming the grid G are parallel to the radiation, the radiation reaches the imaging apparatus 100B without being blocked by the thin sheets Ga. Therefore, the radiographic image Ir taken by the imaging apparatus 100B is dark as a whole.

On the other hand, when the focal point F of the radiation approaches the imaging apparatus 100B as shown in FIG. 16B, the radiation reaches the imaging apparatus 100B without being blocked by the thin plate Ga in the portion close to the center of the radiation incident surface where there is not much difference between the direction of the radiation and the direction of the thin plate Ga, such that the center portion of the radiographic image Ir becomes dark. However, the radiation is blocked by the thin plate Ga and becomes difficult to reach the imaging apparatus 100B at the portion close to the edge of the imaging apparatus 100B, where the difference between the direction of the radiation and the direction of the thin plate Ga is large, such that the peripheral portion of the radiographic image Ir becomes whiter than the central portion. The degree to which the radiographic image Ir becomes white changes depending on the degree of change in the distance between the focal point F of the radiation and the imaging apparatus 100B from the predetermined distance.

The current SID can be calculated on the basis of this principle, using the radiation arrival amounts measured from the density at the central portion and the density at the peripheral portion of the radiographic image Ir.

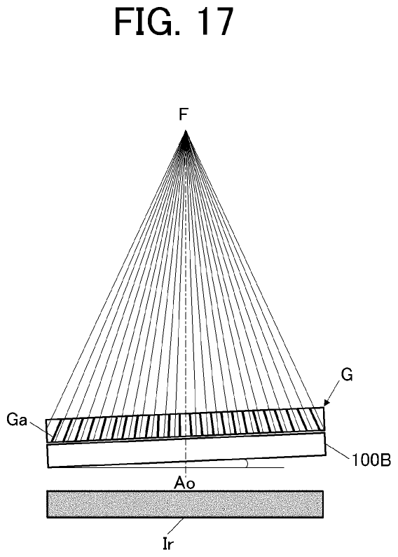

Further, as shown in FIG. 17, when the radiation incident surface of the imaging apparatus 100B is inclined, the radiation is blocked by the thin plates Ga and becomes difficult to reach the imaging apparatus 100B, such that the radiographic image Ir becomes white as a whole. The degree to which the radiographic image Ir becomes white changes depending on the degree of inclination of the radiation incident surface relative to the optical axis Ao of the radiation.

The current imaging apparatus arrangement angle θ_{diff} can be calculated on the basis of this principle, using the density of the radiographic image Ir as a whole.

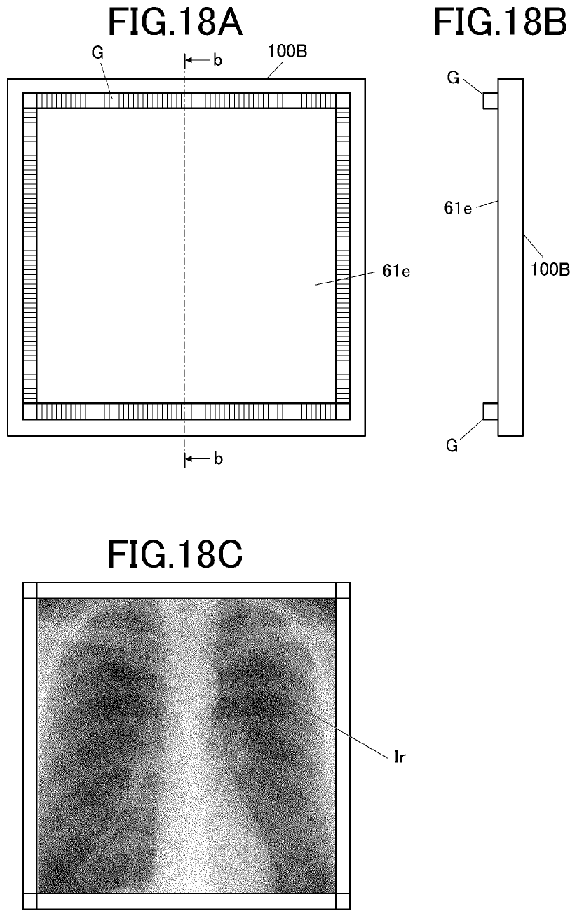

As shown in FIGS. 18A and 18B, the grid G is desirably arranged as narrow as possible at a position facing the peripheral portion of the radiation incident surface 61e in order that the grid G has a width sufficient for detection of the SID and the imaging apparatus arrangement angle θ_{diff}, and does not block radiation to the imaging region.

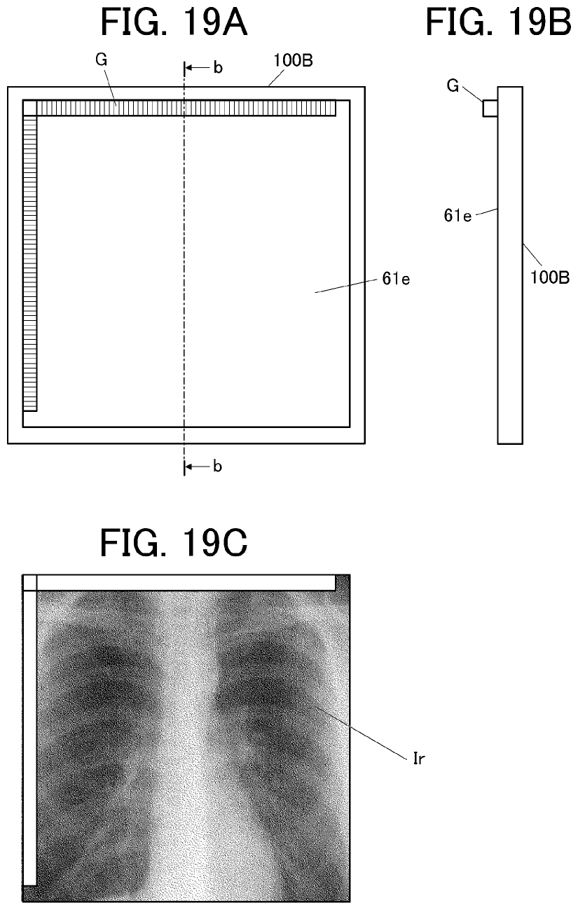

The grid G may have a rectangular shape having sides each facing each of the four sides of the radiation incident surface as shown in FIG. 18A, or may have an L shape facing the two sides of the radiation incident surface as shown in FIG. 19A.

Since the image of the subject S is also superimposed on the image of the grid G, the current SID or imaging apparatus arrangement angle θ_{diff} may be detected by imaging when the subject S does not exist.

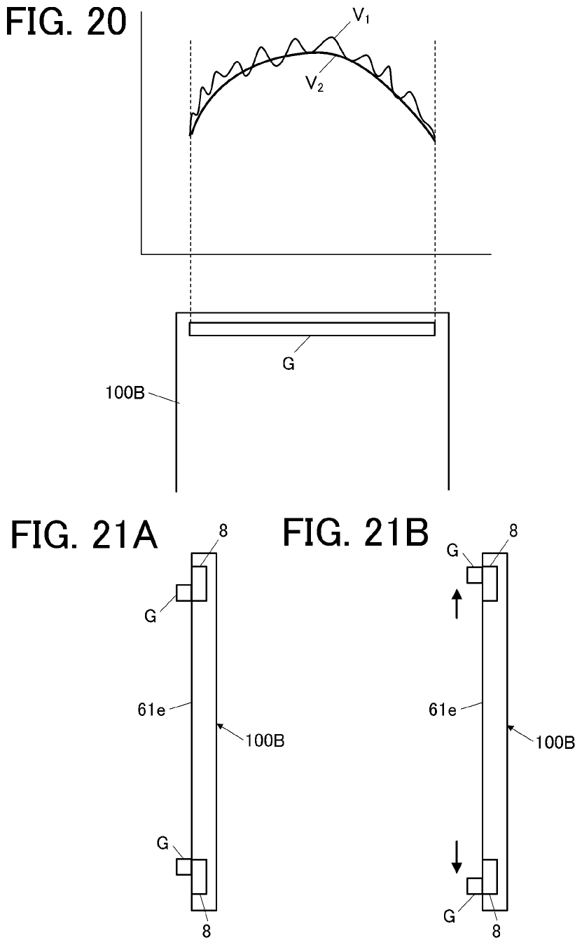

Further, since the image of the subject S is also superimposed on the image of the grid G, the signal value V₁ of the imaging region of the grid G is subjected to smoothing processing to generate a processed signal value V₂ as shown in FIG. 20, on the basis of which the current SID or imaging apparatus arrangement angle θ_{diff} is detected.

Further, an actuator 8 or the like capable of moving the grid G may be provided, such that the grid G may be evacuated to a region not facing the radiation incident surface 61e during imaging as illustrated in FIGS. 21A and 21B.

The diagnostic image may be taken with the grid G, and the current SID or imaging apparatus arrangement angle θ_{diff} may be determined from the diagnostic image.

The current SID and imaging apparatus alignment angle θ_{diff} may be calculated from the density of the image acquired by irradiating the grid G with weaker radiation before taking a diagnostic image than in taking a diagnostic image, and adjusted on the basis of the calculated values.

### [Measurement Method (5)]

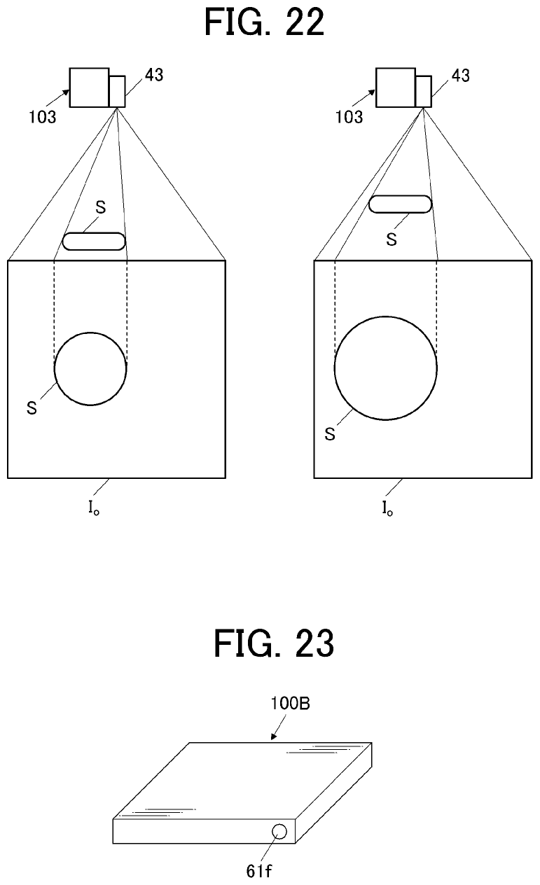

In view of the problem that it is difficult to grasp the SID and adjust it to a predetermined SID according the conventional radiation imaging system, an optical camera 43 which acquires optical images in the emission direction of the radiation may be provided at the radiation emitter 103, at a portion from which relative position to the radiation emitter 103 does not change, or at a portion from which the relative position can be detected as shown in FIG. 22. Also, there may be provided a controller which calculates the current SID based on the size of the subject S or the imaging apparatus 100B in the optical image Io.

In this case, the size of the subject S or imaging apparatus 100B is input to the console 4 or the like in advance, and the imaging magnification of the optical camera 43 is unchanged.

In this way, the current SID can be grasped, and imaging can be performed with an appropriate SID.

Moreover, the exposure dose of the subject S can be reduced because it is not necessary to emit radiation before imaging for grasp of the SID as in the above Measuring Method (4).

When the optical camera 43 is a monocular camera, only one camera is required. Further, image processing can be performed more easily than when it is a compound eye camera.

Further, calibration can be simply performed when a monocular camera is used.

Further, if the relationship between the imaging magnification and the imaging range is known in advance, the magnification for imaging by the optical image acquisition means may be varied.

Further, distortion may be corrected in the range taken by the optical camera 43.

### [Calibration Method]

According to the calibration method in the above embodiment, the imaging apparatus 100B is necessarily arranged at a specific position at a known height. Therefore, there is a problem that an error may occur due to fluctuations in atmospheric pressure or the like from arrangement of the imaging apparatus 100B at the specific position to actual imaging

Further, if imaging is performed after the imaging apparatus 100B is moved to a place having a height different from the place where calibration is performed (for example, another floor in a three-story building), there is a risk that a wrong SID may be displayed if calibration is not performed after the movement.

Even when the imaging apparatus 100B is fixed and used, due to atmospheric pressure which fluctuates significantly when an anticyclone or a depression passes, there is a risk that a wrong SID may be displayed without frequently repeated calibration.

In view of such a problem, the height of the detected imaging apparatus 100B may be corrected on the basis of a measurement value measured by a second air pressure sensor 67A which is provided at the above specific portion of the movable vehicle 100A measures the atmospheric pressure at its own height.

In this way, the height of the imaging apparatus 100B relative to the specific portion of the movable vehicle 100A can be calculated based on the measurement values by the air pressure sensor 67 and the temperature sensor and the measurement value of the second air pressure sensor 67A in the imaging apparatus 100B (on the assumption that the temperature around the movable vehicle 100A is the same as the temperature around the imaging apparatus 100B).

That is, since the second atmospheric pressure sensor 67A arranged in the movable vehicle 100A can always cancel out fluctuations in the atmospheric pressure, calibration processing is not required.

### [Calibration Switch]

In the height measurement of the imaging apparatus 100B using air pressure sensor 67 according to the first embodiment described above, it is necessary to perform calibration at appropriate timing in response to the height change due to air pressure change around the imaging apparatus 100B or radiation imaging system 100, movement to another floor, etc. However, if the calibration is performed at a timing not intended by the user, the display of the SID or the like may change at a timing not intended by the user, which may cause a problem.

In view of such problems, as shown in FIG. 23, for example, the imaging apparatus 100B may be provided with a button 61f operated by the user for calibration start, so that calibration may be started at the timing when this button is operated.

In this way, calibration can be performed at the timing intended by the user, and the problem that the display of the SID or the like changes at a timing not intended by the user can be reliably avoided.

The switch 61a or 61b may be used as the calibration start button. In this case, calibration may start when the switch 61a or 61b is operated in a different manner than usual (for example, pressed for a long time or double- clicked).

Also, the button may not be provided on the imaging apparatus 100B, but may be provided on the system main body 100A (for example, on the display 41).

When calibration is started, the start may be displayed or output by voice so that the user is notified of it.

Furthermore, an OK button may be provided separately from the calibration start button. Even if the calibration start button is operated, calibration does not start until the OK button is operated (i.e., lock function to prevent accidental operation).

### [Air Pressure Sensor Arrangement Position]

The imaging apparatus 100B has an airtight structure which prevents infiltration of liquid such as blood. For this reason, if the imaging apparatus 100B incorporates the air pressure sensor(s) 67 in the first embodiment described above, the air pressure sensor 67 is arranged in the inner space Sp₁ in the airtight structure of the imaging apparatus 100B. As a result, the air pressure outside the imaging apparatus 100B cannot be accurately measured.

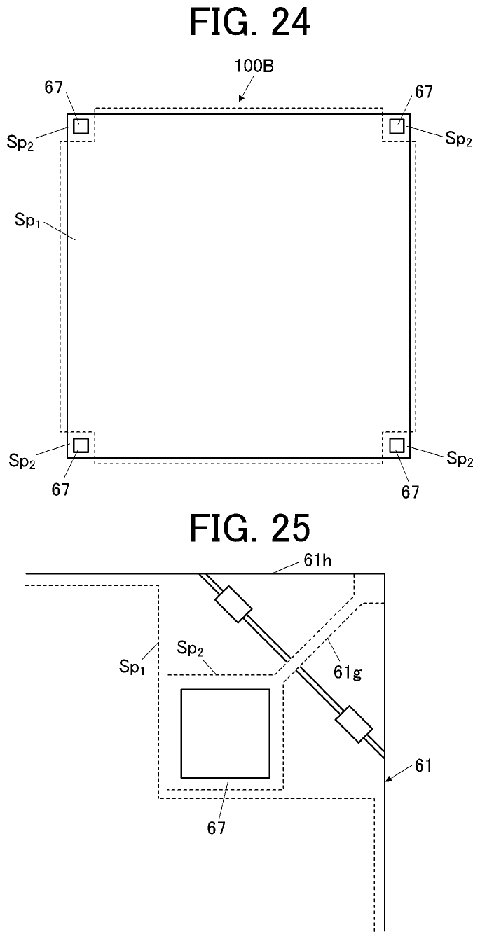

In view of such a problem, for example, as shown in FIG. 24, the imaging apparatus 100B may be provided with intermediate spaces Sp₂ which are not in the airtight structure. The air pressure sensors 67 may be arranged in intermediate spaces Sp₂ which are inside the imaging apparatus 100B and outside the airtight space Sp₁.

In this way, even when the air pressure sensor(s) 67 are incorporated in the imaging apparatus 100B, the air pressure around the imaging apparatus 100B can be accurately measured.

### [Air Pressure Sensor Arrangement Structure]

When the air pressure sensor 67 is placed outside the confidential structure as described above, liquid such as blood may be put on the air pressure sensor 67 and make it unavailable, so that calculation of SID etc. becomes impossible.

In view of such a problem, a thin communication path 61g may be detachably formed to connect the intermediate space Sp₂ of the imaging apparatus 100B and the outer space.

Specifically, as shown in FIG. 25, the air pressure sensor 67 is placed in the housing 61 of the imaging apparatus 100B, and a removal portion 61h has the communication passage 61g which connects the intermediate space Sp₂ (outside of the inner space Sp₁ in the airtight structure) and the space outside the housing 61.

A specific method of making the removal portion 61h detachable from the housing is, for example, a snap fit method in which a recess is formed in one of the housing 61 and the removal portion 61h, and a protrusion fitting the recess is formed in the other.

The width of at least a part of the communication passage 61g is narrowed in the housing 61 and/or in the removal portion 61h.

In this way, even if liquid is put on the imaging apparatus 100B, the liquid stops at the narrow portion of the communication passage 61g. Therefore, air pressure sensor 67 can be prevented from becoming unavailable due to the liquid reaching the air pressure sensor 67.

Further, even if the liquid blocks the communication passage 61g and cannot be removed, the air pressure can be measured again by replacing the removal portion 61h.

An end of the communication passage 61g in the removal portion 61h may be an air intake portion having wider width than the middle portion of the communication passage 61g.

The width of either a housing-side end of the communication passage 61g in the removal portion 61h or the opening of the intermediate space Sp₂ provided with the air pressure sensor 67 in the housing may be expanded so that a connecting portion is formed in order that the communication passage 61g of the removal portion 61h and the opening of the housing are easily connected.

In at least a part of the communication passage 61g in the housing 61 and/or in the removal portion 61h, there may be formed a folded portion which makes the liquid not flow easily, a liquid reservoir having intentionally expanded width, and the like.

Further, at least a part of the communication passage 61g may be formed of a hydrophobic material. Alternatively, a part of the communication passage 61g may be formed of a hydrophilic material.

Further, an absorber that absorbs liquid may be arranged on at least a part of the wall surface of the communication passage 61g.

If an abnormality is detected in the value measured by the air pressure sensor 67, such as no change for a predetermined period in the value measured by the air pressure sensor 67, a notification indicating that an abnormality has been detected or a notification prompting replacement of the removing unit 61h may be performed.

### [Arrangement of Air Pressure Sensor in Holder]

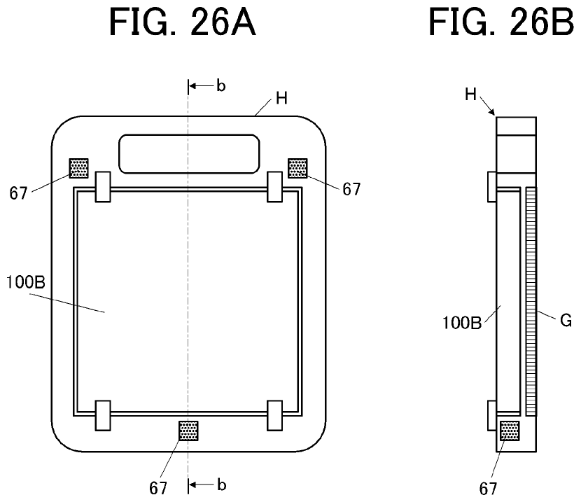

Also, in view of the problem that, if the imaging apparatus 100B incorporates the air pressure sensor(s) 67 in the first embodiment described above, the air pressure sensor 67 is arranged inside the airtight structure of the imaging apparatus 100B, as shown in FIGS. 26A and 26B, for example, the air pressure sensors 67 may be arranged in a holder H including the imaging apparatus 100B.

In this way, since the holder H does not have to be airtight, the air pressure can be accurately measured, and the height can be accurately calculated from the measured air pressure.

The holder H may include a battery, a controller for calculating the height from the measurement value, and a communication unit for transmitting the calculated height to the outside. In this way, the air pressure can be measured by the holder H alone.

At that time, the air pressure measured by the air pressure sensor 67 in the holder H or the height calculated by the controller in the holder H may be transmitted to the system main body 100A directly or via the imaging apparatus 100B. In this way, height information can be transmitted only by performing short-distance data communication between the holder H and the imaging apparatus 100B. Therefore, energy required for communication can be reduced.

The electric power used in the holder H may be supplied from the imaging apparatus 100B. At that time, the electric power may be supplied by wire via the connector of the imaging apparatus 100B and that of the holder H which is formed where the connector of the imaging apparatus 100B is engaged. The electric power may be supplied wirelessly using an electromagnetic action or the like.

Further, as shown in FIG. 26B, the holder H may be provided with the grid G.

### [Use of Sensor]

In serial imaging, the subject S may move and not be in a desired imaging state in the period between the positioning of subject S and the start of the imaging by the user, or during the imaging.

Further, it may be difficult to determine on the basis of the taken image alone whether the motion of the subject S is a specific body motion to be diagnosed or a body motion other than the specific body motion not to be diagnosed.

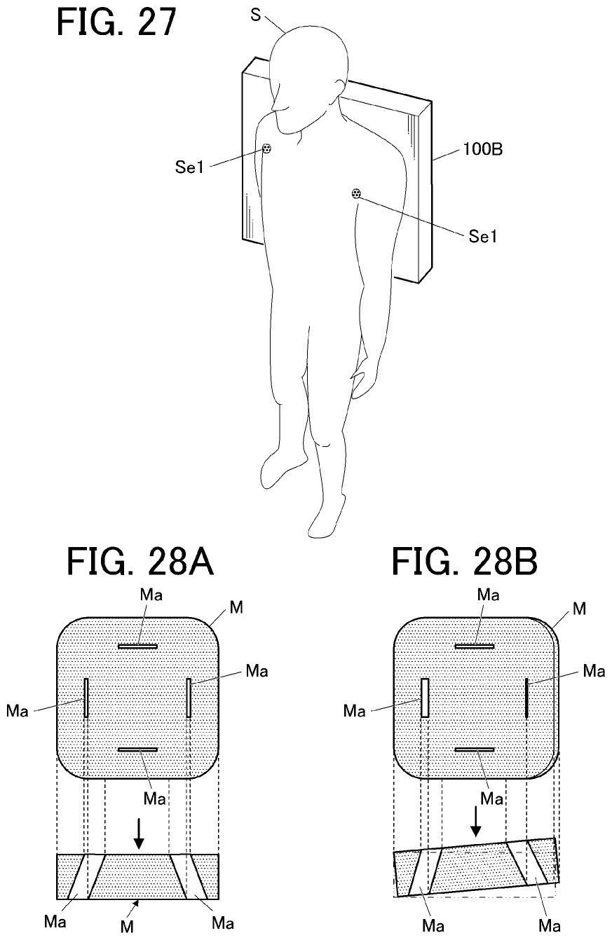

In view of such a problem, as shown in FIG. 27, for example, a sensor Se1 for detecting the motion of the subject S is attached to the subject S, of whom serial imaging is performed on the basis of an arithmetic formula depending on the imaging technique, the value measured by the sensor Se1 and transmitted to the console 4, and the like, it is determined whether or not the measurement value from the sensor Se1 results from a body motion other than the specific body motion, that is, not to be diagnosed, whether or not the image is difficult to be used for diagnosis because of too large body motion other than the specific motion, and the like.

The specific method for attaching the sensor Se1 includes, for example, sticking with adhesive.

Examples of the attached sensor Se1 include an acceleration sensor, an angle sensor, a gyro sensor, a geomagnetic sensor, and the like.

When the console 4 determines that the image is difficult to be used for diagnosis because of the too large body motion, the user is notified of the determination or radiation emission and imaging are stopped.

In this way, it is possible to grasp the degree of body motion other than the specific body motion based on the value measured by the sensor Se₁. Then, if the motion is so large that the image cannot be used for diagnosis, notification to the user and cancellation of imaging are performed. Thus, the risk that the subject S is unnecessarily exposed to a radiation can be reduced.

The motion information detected by the sensor Se₁ may be transmitted to the system main body 100A directly or via the imaging apparatus 100B.

### [Use of Marker]

If imaging is performed with the marker M attached to the subject S, the state of the subject S can be recognized from the taken image of the marker M, which has a relatively simple shape such as, for example, a cylindrical shape. Since such a marker M looks the same shape when the subject S is inclined to the right or to the left, the direction of the imaging apparatus arrangement angle θ_{diff} could not be detected. As a result, there is a problem that the subject S cannot be properly instructed in which direction to tilt.

In view of such a problem, the marker M to be used may have a hole Ma penetrating the marker M in a direction not vertical to the surface to be attached to the subject S of the marker M.

Specifically, for example, as shown in FIG. 28A, holes Ma penetrating toward the surface to be attached are formed at four portions (top, bottom, left, and right) when viewed from the radiation emission direction. Each of the holes Ma is inclined away from the center of the marker M as it goes to the surface to be attached. Although only the cross-sectional view in the left-right direction of the marker M is shown in FIG. 28A, the cross-sectional view in the vertical direction is the same.

When the marker M is irradiated with radiation, the radiation reaching the subject S and the imaging apparatus 100B is attenuated while passing through the region other than the holes Ma. Meanwhile, the radiation reaching the subject S and the imaging apparatus 100B is not attenuated while passing through the holes Ma, and thus more than the radiation passing through the region other than the holes Ma.

In a general radiographic image, portions strongly attenuating radiation appear white, and those weakly attenuate radiation appear black. Therefore, the portion of the marker M other than the holes Ma appears white, and the portions of the holes Ma appear black.

Here, when the marker M is arranged so as to have a surface to be attached orthogonal to the optical axis Ao of the radiation, the holes Ma formed on the left and right sides of the marker M are inclined in opposite directions with respect to the optical axis Ao of radiation, but by the same inclination angles. Therefore, the widths of the holes Ma (slits) on the left and right sides of the marker M viewed from the focal point F side of the radiation appear to be equal as shown in FIG. 28A. Therefore, as shown in FIG. 28A, the widths of the holes Ma (slits) on the left and right sides of the marker M viewed from the focal point F of the radiation appear to be equal.

Meanwhile, when the marker M is arranged so as to have a surface to be attached inclined by θ degrees with respect to the optical axis Ao of the radiation, the holes Ma formed on the left and right sides of the marker M are inclined with respect to the optical axis Ao of radiation by different inclination angles from each other. Therefore, as shown in FIG. 28B, the widths of the holes Ma (slits) on the left and right sides of the marker M viewed from the focal point F side of the radiation appear to be different from each other. Specifically, the hole Ma at the side to which the marker M is inclined appears to be large. The width of the hole Ma varies in proportion to the angle θₘ by which the surface to be attached of the marker M inclines with respect to the optical axis Ao.

On the basis of this principle, it is possible to calculate how much and in which direction the marker M is inclined.

In this way, the inclination direction of the subject S to which the marker M is attached can be estimated according to the inclination direction of the marker M. Then, the subject S can be adjusted to face in a direction suitable for imaging

As the material of the marker M, a material having a large attenuation coefficient of radiation (for example, metal, magnet, and the like) is desirably used, so that the edge portion of the hole Ma can be clearly recognized.

However, in some cases, a material having a small radiation attenuation coefficient (for example, resin, wood, and the like) is desirably used as the marker M. Such a material makes it possible to take the image of the subject S at the position of the marker easily, if the image at the marker is also desirably used for diagnosis.

On the basis of image processing for recognition of the marker in the image, further image processing such as contrast change of the image at the marker may be performed such that the marker on the image is visually recognized less.

Further, the inclination direction and the inclination angle may be calculated based on the taken image of the marker M in the system main body 100A or in the image processor of the imaging apparatus 100B.

### [Subject Motion Detection]

In view of the problem that, in serial imaging, the subject S may move and not be in the desired imaging state in the period between the positioning of subject S and the start of the imaging by the user, or during the imaging, for example, an optical camera 43 and an actuator 103b may be provided as shown in FIG. 29, and the console 4 or the imaging controller may be configured to detect motion of the subject S by image processing of the optical image Io of the subject S taken by the optical camera 43 and to control the actuator 103b in accordance with the detected motion of the subject S. The optical camera 43 is provided at the radiation emitter 103, at a portion from which relative position to the radiation emitter 103 does not change, or at a portion from which the relative position can be detected, and performs imaging of the subject S in the radiation emission direction. The actuator 103b is provided at the arm 102 or at a connecting portion between the arm 102 and the radiation emitter 103, and controls the radiation direction.

In this way, even when the subject S moves, the position of the radiation emitter 103 can be controlled in response to the detected motion of the subject S so that the desired imaging state is kept.

The user may be notified of the correction direction and correction amount of the radiation emitter 103 position (assisted in adjustment) based on the motion of the detected subject S without the actuator 103b or the configuration of controlling the actuator 103b.

Not only the direction but also the angle may be detected as the motion of the subject S.

If it is difficult to detect the motion of the subject S based on image processing, the motion of the subject S may be estimated based on image processing for detection of the motion of marker M attached to the subject S.

[Use of camera]

In the case of imaging of a standing subject S or imaging in a room for imaging, it is relatively easy to adjust the positions of the radiation emitter and imaging apparatus.

However, in the case of imaging using the movable vehicle 100A, there are many cases of imaging the subject S in a decubitus position (for example, in a face-up position on a bed or in a position facing obliquely to the upper surface of the bed) who is difficult to get up and move. In this case, since the radiation emitter 103 needs to be arranged at a high position and emit radiation downward, it is difficult to confirm the positions of the radiation emitter 103 and the imaging apparatus 100B from the radiation emitter 103.

In particular, if the user who performs imaging is short of stature (for example, a female engineer or the like), the confirmation becomes more difficult.

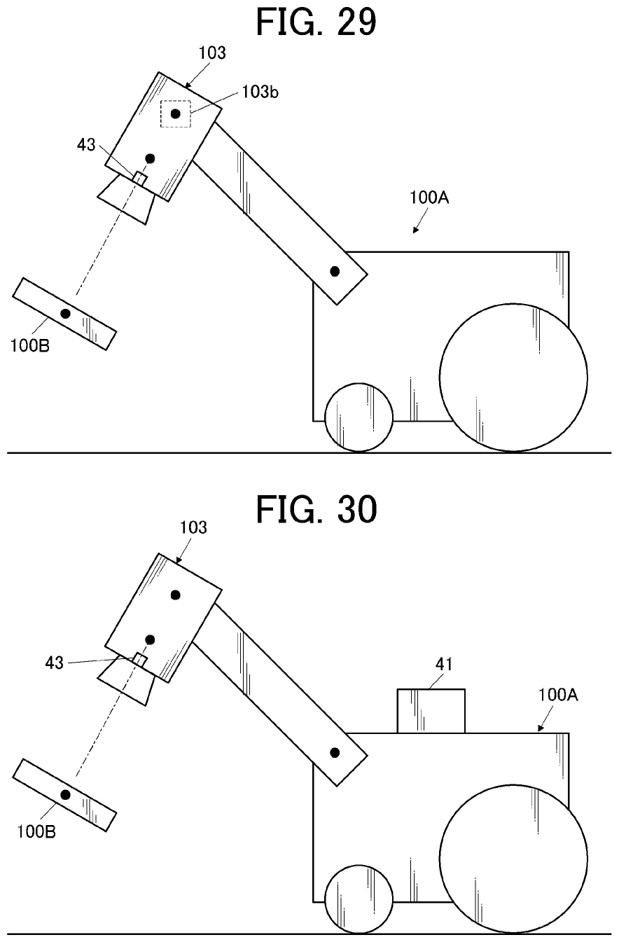

In view of such a problem, for example, an optical camera 43 and a display 41 may be provided as shown in FIG. 30. The optical camera 43 may be provided at the radiation emitter 103, at a portion from which relative position to the radiation emitter 103 does not change, or at a portion from which the relative position can be detected, and performs imaging of the subject S in the radiation emission direction. The display 41 displays the image taken by the optical camera 43.

In this way, the user can adjust the positions of the imaging apparatus 100B, the subject S, and the radiation emitter 103 viewed from the radiation emitter 103 displayed on the display 41, which improves the workability.

Further, it is possible to reduce the risk of taking radiographic images that can not be used for diagnosis due to the imperfect alignment of the imaging apparatus 100 B, subject S, radiation emitter 103.

### [Use of Pressure Sensor (1)]

In imaging of the subject S in a decubitus position, the imaging apparatus 100B is disposed between the bed and the subject S. Therefore, it is difficult to visually recognize the imaging apparatus 100B from the radiation emitter 103 side, and to grasp the relative position between the subject S and the imaging apparatus 100B. As a result, it was difficult to position the subject S to be in a desired position.

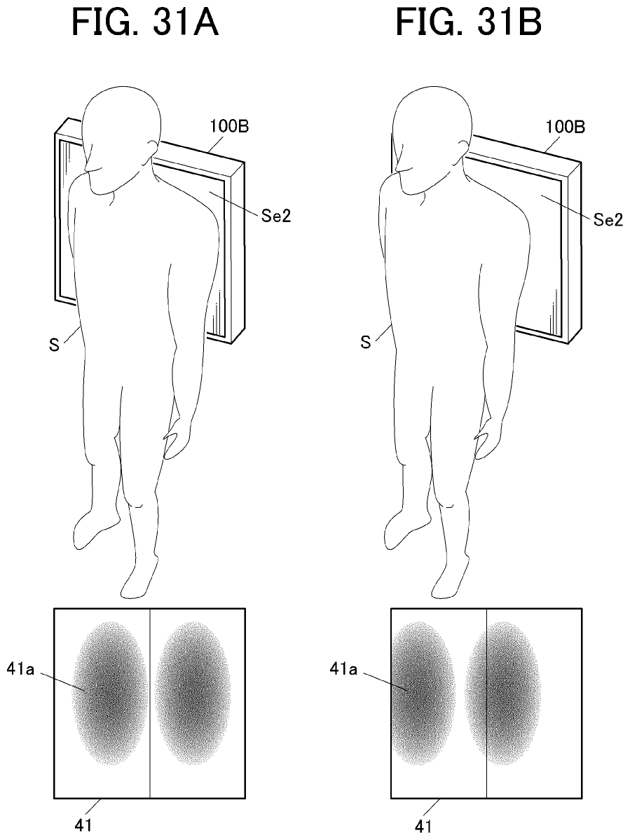

In view of such problems, as shown in FIGS. 31A and 31B, for example, a pressure sensor Set which measures the pressure applied to the imaging apparatus 100B and a display 41 which displays the measurement result by the pressure sensor Set may be provided.

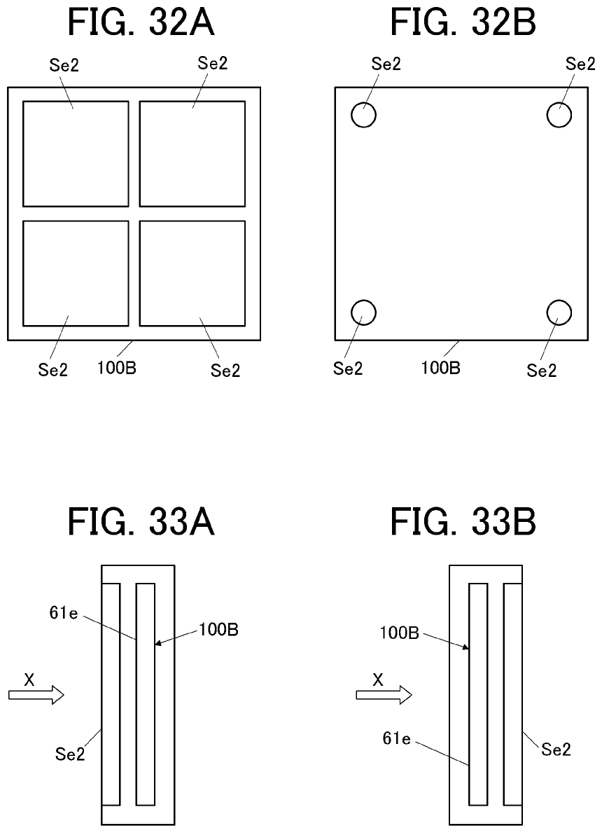

The pressure sensor Set may be a planar pressure sensor which measures an in-plane distribution of pressure and is disposed in parallel with the radiation incident surface 61e as shown in FIG. 32A, or may be a plurality of pressure sensors arranged in an array along a radiation incident surface as shown in FIG. 32B.

The radiation incident surface 61e of the imaging apparatus 100B may be divided into a plurality of regions (into vertical directions, horizontal directions, or both directions) in each of which the pressure sensor Set is arranged so as to detect the pressure applied to each region.

Also, the pressure sensor Set may be arranged to detect the pressure at a specific portion.

Also, the pressure sensor Set may be provided so as to face the radiation incident surface 61e of the imaging apparatus 100B as shown in FIG. 33A, for example, or to face the surface opposite to the radiation incident surface 61e of the imaging apparatus 100B as shown in FIG. 33B.

If the pressure sensor Set is provided so as to face the radiation incident surface 61e of the imaging apparatus 100B, the pressure sensor Set can be arranged on the imaging apparatus 100B directly touched by the subject S. Therefore, it is possible to measure the pressure fluctuation in response to the motion of the subject S more quickly and more sensitively. Moreover, it becomes possible to grasp the position of he subject S more promptly and correctly.

If the pressure sensor Set is provided so as to face the surface opposite to the radiation incident surface 61e of the imaging apparatus 100B, the radiation detector 63 accumulates charges corresponding to the radiation transmitted through the pressure sensor Set. Therefore, depending on the material and/or structure of the pressure sensor Set, the pressure sensor Set may visually recognized in the radiographic image Ir. However, because the pressure sensor Set appears in the radiographic image even in the absence of the subject S in such a case, the pressure sensor Set can be removed from the image by, for example, image processing including acquisition of a radiographic image Ir of the pressure sensor Set in advance, before taking an image of the subject S, followed by subtraction of the image of pressure sensor Set from the radiographic image of the subject S, and the like.

Meanwhile, if the pressure sensor Set is provided so as face the surface opposite to the radiation incident surface 61e of the imaging apparatus 100B, the imaging apparatus 100B can take an image using radiation that does not pass through the pressure sensor Set. Therefore, it is not necessary to remove the the visually recognized pressure sensor Set described above.

In this way, it is possible to display on the display 41 the distribution 41a of measurement values by the pressure sensor Set represented by ellipses as shown in the lower part of FIG. 31A and FIG. 31B, for example, or the difference between the measurement values of the pressure sensors Set in several regions and specific regions. From such information, it can be detected to which part of the imaging apparatus 100B the subject S applies pressure.

Further, according to the information on where the subject S applies pressure, it is possible to estimate at which position the subject S is with respect to the imaging apparatus 100B. Then, the subject S can be positioned at desired a position.

In the description so far, the imaging procedure has been described in the case where the subject S is preferably arranged so that the body axis (rostro caudal axis) of the subject S passes through the center point of the radiation incident surface 61e. However, the present embodiment is also applicable to imaging procedures other than such a case.

For example, in an imaging procedure where the subject S is preferably arranged so that the body axis of the subject S passes a reference point laterally shifted from the center of the radiation incident surface 61e by a specified distance (for example, two thirds on the right), it is determined whether the reference point is located between two ellipses representing the distribution of measurement values as shown in FIG. 31B.

Also, the radiation imaging system may be configured to have a function to monitor the value of the pressure sensor Set during imaging and, when a change in pressure sensor Set value is detected, a function to notify the user of possibility that there may be a change in the positional relationship between the imaging apparatus 100B and the subject S by sound, light, display, and the like or a function to stop radiation emission and stop imaging.

In this way, it is possible to detect that desired imaging may not have been performed in serial imaging due to change in positional relationship between the imaging apparatus 100B and the subject S as a result of movement of the imaging apparatus 100B or subject S (for example, a slip of the imaging apparatus 100B).

### [Use of Length Measurement Device]

In imaging preparation, in imaging of still images, or in serial imaging, positioning of the subject S is necessary to a desired position of the imaging apparatus 100B.

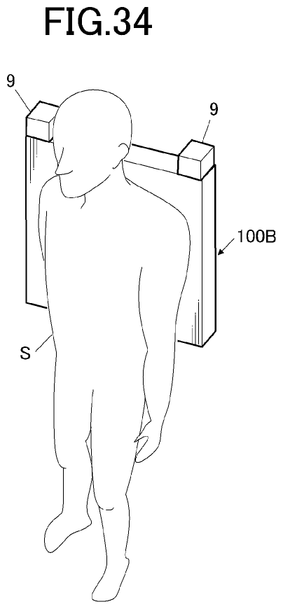

In view of such a problem, for example, as shown in FIG. 34, a length measurement device 9 may be provided at the end of the imaging apparatus 100B.

In this way, it is possible to determine whether or not the subject S is positioned at the desired position relative to the imaging apparatus 100B, and to perform imaging while the subject S is suitably positioned.

The length measurement device 9 may be provided on one side of the imaging apparatus 100B to detect the position of the subject S based on a distance from the one side, or may be provided on both sides of the imaging apparatus 100B to detect the position of the subject S based on distances from the both sides. If two length measurement devices 9 similar to each other are provided on both sides of the imaging apparatus 100B, the position of the subject S can be detected more accurately. In particular, it is effective when the subject S is preferably positioned at the center of the imaging apparatus 100B.

The length measurement device 9 may be, for example, a tape measure that can be pulled out from the housing, or a non-contact type using a laser. In measurement with a non-contact length measurement device 9 using a laser, which does not require the user to use his hand, length measurement can be continued during serial imaging so that, it can be confirmed whether or not the subject S is not moving from the preferred imaging state during serial imaging.

If it is determined that the subject S is moving as a result of length measurement, the user may be alerted, radiation emission and imaging may be stopped, and the like.

### [Use of Proximity Sensor]

In view of the problem that positioning of the subject S is necessary to a desired position of the imaging apparatus 100B in imaging preparation, in imaging of still images, or in serial imaging, the imaging apparatus 100B may be provided with a proximity object detection sensor Se3 or a contact detection sensor Se4 at its end, and the radiation imaging system may have a function of judging the presence or absence of an object in proximity to the proximity object detection center or the presence or absence of an object contacting the contact detection sensor.

A capacitance type sensor may be used as the proximity object detection sensor Se3, for example.

The contact object detection sensor Se4 to be used may be a sensor employing a resistive film method, an acoustic pulse recognition method, an ultrasonic surface acoustic wave method, an infrared light shielding method, an electrostatic capacitance method, a surface electrostatic capacitance method, a projection electrostatic capacitance method, an electromagnetic induction type sensor, or the like.

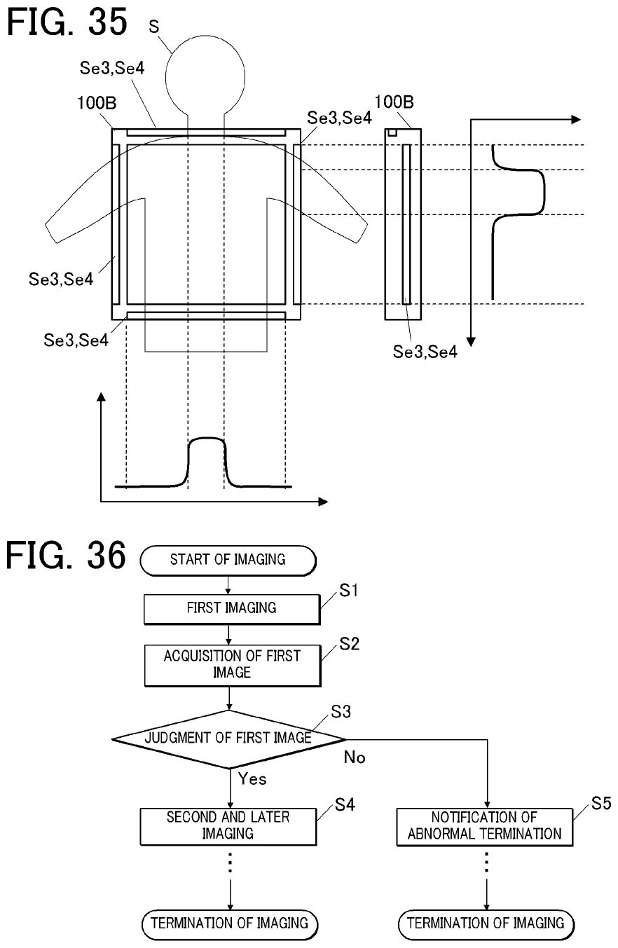

As shown in FIG. 35, proximity object detection sensors Se3 or contact object detection sensors Se4 each extends linearly and are desirably arranged at the edges of the imaging apparatus 100B.

In such a case, each of the sensors Se3 or Se4 may have a plurality of regions along the extension direction, such that each of the regions can detect proximity or contact of an object.

In this way, it is possible to judge the position which an object is in proximity to or in contact with in the imaging apparatus 100B or whether or not an object is in proximity to or in contact with a desired position (for example, at the central position of the imaging apparatus 100B).

The sensors Se3 and Se4 may notify the user of detection results for each region.

[Positioning Confirmation based on Taken Image]

In view of the problem that positioning of the subject S is necessary to a desired position of the imaging apparatus 100B in imaging preparation, in imaging of still images, or in serial imaging, whether or not the subject S is positioned at a desired position may be confirmed using the first image taken in serial imaging.

Specifically, for example, imaging is performed according to the flow shown in FIG. 36. First, first imaging is performed (step S1) to acquire the first image (step S2). Then, it is judged whether or not the subject S is positioned at a desired position (step S3). If it is judged in step S3 that the subject S is positioned at the desired position (step S3; Yes), the second and later images are continually taken (step S4), and the imaging process is terminated. On the other hand, if it is judged in step S3 that the subject S is not positioned at the desired position (step S3; No), the judgement result notification is made (step S5), and/or the imaging is stopped as necessary.

In the judgement on whether or not the subject Sin step S3 is positioned at a desired position, the judgement method or judgement condition may be varied for each imaging procedure.

For example, it is possible to judge whether or not the subject S is positioned at the center of the image by judging whether the image is symmetrical or not using a part of or all of the images. It is also possible to judge whether the subject S is positioned at a desired position or not by comparing the taken image with an image which is taken stored in advance with the same imaging technique and includes the same composition as the taken image.

If the subject S has moved from the desired position before start of the imaging, it may not be possible to acquire an image that can be used for diagnosis even by imaging. However, according to the present embodiment, if it is judged that the image cannot be used for diagnosis, the user is notified that the subject S has moved from the desired position. In response to the notification, the user confirms whether or not the taken image can be used for diagnosis, and stops imaging if it cannot be used for diagnosis. This prevents the subject S from being exposed to an unnecessary radiation

If imaging is automatically stopped when it is judged that the subject S has moved from the desired position, it is possible to further prevent the subject S from being exposed to an unnecessary radiation.

If information between adjacent frame images acquired by serial imaging is important, a judgement may be made on the basis of not only the first image but a plurality of images from the first one until it becomes possible to judge information between adjacent frame images.

In this way, it is possible to accurately determine whether or not the image can be used for diagnosis even in imaging procedures where the difference between a plurality of adjacent frame images is important for diagnosis.

If it takes time for image processing etc. and the judgement is not in time for the second imaging, a judgement of the first image may be performed in parallel with the second and later imaging

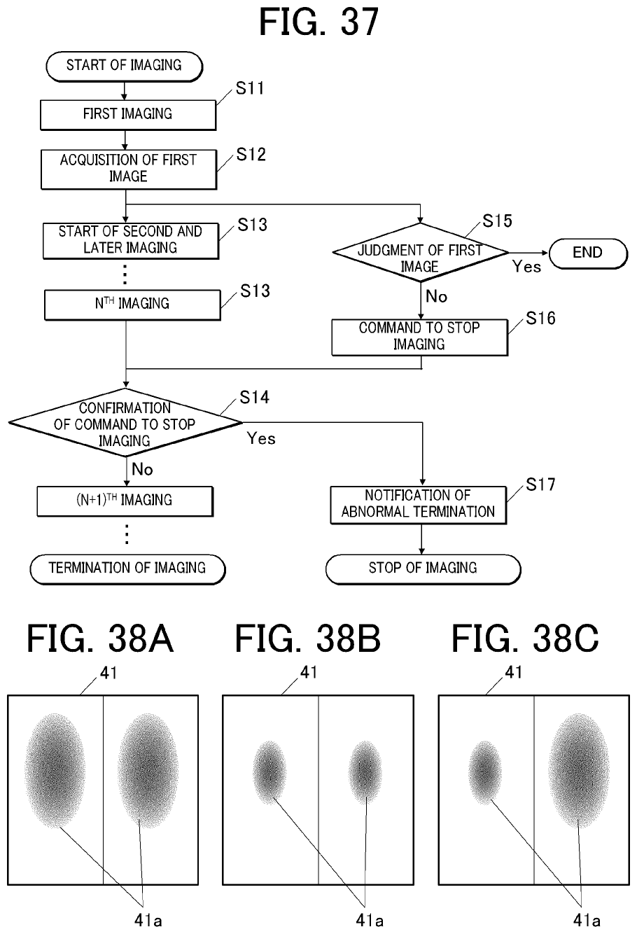

Specifically, for example, imaging is performed according to the flow shown in FIG. 37. First, first imaging is performed (step S11) to acquire the first image (step S12). Subsequently, in parallel with second and later imaging (step S13) and confirmation of command to stop imaging (step S14) alternatively performed, a judgement is made whether or not the subject S is positioned at a desired position (step S15). If it is judged in step S15 that the subject S is positioned at the desired position (step S15; Yes), the imaging process is terminated. In this way, serial imaging is performed to the end. On the other hand, if it is judged in step S15 that the subject S is not positioned at the desired position (step S15; No), a command to stop imaging is output (step S16). Then, as the command to stop imaging is confirmed in step S14, notification of the confirmation result is made (step S17), and the imaging is terminated as necessary.

As a result, even if it takes time for image processing, second and later imagings are continuously performed without delay. At the same time, when it is determined that the image is not available for diagnosis, motion of the subject S from the desired position is notified to the user. In response to the notification, the user confirms whether or not the images being taken can be used for diagnosis and stops imaging when it cannot be used for diagnosis. This prevents the subject S from being exposed to an unnecessary radiation.

If imaging is automatically stopped when it is judged that the subject S has moved from the desired position, it is possible to further prevent the subject S from being exposed to an unnecessary radiation.

Although the judgement is made based on the first image in serial imaging in the above description, it may be made based on the second or later image instead of the first image.

For example, the image acquired in the first imaging may not be suitable for the judgement, as the radiation is also emitted for the first time and is unstable. In such a case, the judgement may be made not in the first imaging but in the imaging after the emission of radiation becomes stable.

Depending on the imaging procedure, the subject S may cause acceptable body motion. For example, when the subject S breathes, body motion corresponding to breathing occurs. For a judgement at a specific timing in such acceptable motion, as described above, a judgement may be made not on the basis of the first image but on the image taken corresponding to the specific timing according to the imaging procedure.

### [Use of Pressure Sensor (2)]

In the imaging, the subject S is required to be in firm contact with the imaging apparatus 100B. However, since the imaging apparatus 100B is placed on the back of the subject S, it was difficult to confirm whether the subject S is in firm contact with the imaging apparatus 100B. As a result, there was a problem that a desired radiographic image can not be obtained because the user takes an image without being aware that the subject S is separated from the imaging apparatus 100B.

In particular, in follow-up observation before and after surgery etc. where it is necessary to take images showing changes over time, such as the size of the affected portion, images has to be taken under the same conditions. However, since it is difficult to confirm the contacting state of the subject S and the imaging apparatus 100B as described above, it is difficult to take images under the same conditions.

Further, in serial imaging, the subject S may move and not be in a desired imaging state in the period between the positioning of subject S and the start of the imaging by the user, or during the imaging In particular, in imaging of the subject S lying in bed, the imaging apparatus 100B which is insufficiently fixed is moved by the slight motion of the subject S. As a result, the positional relationship between the subject S and the imaging apparatus 100B may change such that the imaging condition may be out of the desired state.

In view of such a problem, a pressure sensor that measures the pressure applied to the imaging apparatus 100B and a display 41 which displays the measurement result by the pressure sensor may be provided.

The pressure sensor may be a planar pressure sensor which measures an in-plane distribution of pressure and is disposed in parallel with the radiation incident surface, or may be a plurality of pressure sensors arranged in an array along a radiation incident surface.

The radiation incident surface of the imaging apparatus 100B may be divided into a plurality of regions (into vertical directions, horizontal directions, or both directions) in each of which the pressure sensor is arranged so as to detect the pressure applied to each region.

Also, the pressure sensor may be arranged to detect the pressure at a specific portion.

In this way, it is possible to display on the display 41 the distribution of measurement values by the pressure sensor represented by ellipses as shown in FIGS. 38A, 38B, and 38C, for example, or the difference between the measurement values of the pressure sensors in several regions and specific regions. From such information, the user can determine whether or not the subject S is in firm contact with the imaging apparatus 100B, and thereby adjust the contact state of the subject S with the imaging apparatus 100B.

As shown in FIG. 38C, when the left and right pressure values (the size of the ellipse) displayed on the display 41 are different, the subject S may be in contact with the imaging apparatus 100B in a biased state to the left or right. FIG. 38C illustrates the case where the left and right pressure values are different. However, when the upper and lower pressure values are different, the subject S may be in contact with the imaging apparatus 100B in a biased state upward or downward.

In this way, the user can estimate the contact state of the subject S with the imaging apparatus 100B on the basis of the difference between the pressure values, and instruct the subject S to change the contact state in an appropriate direction.

[Confirmation by Comparison with Still Image]

On the basis of a still image acquired before performing serial imaging, whether or not the subject S is positioned at a desired position of the imaging apparatus 100B may be confirmed. However, even if it is confirmed that the subject S is positioned at the desired position on the basis of the still image, there is a problem that desired imaging can not be performed due to motion of the subject S or the imaging apparatus 100B by the start of serial imaging after taking a still image.

In view of such a problem, whether or not the subject S is positioned at a desired position may be confirmed by comparing the first frame image taken during serial imaging with the still image taken and stored in advance.

For example, confirmation can be made with processing in which the processing of step S3 (judgement of the first image) of FIG. 36 is changed to judgement by comparison with a still image taken in advance.

At that time, the determination (confirmation) can be made automatically by, for example, comparison of the images (i.e., correlation between the images) in the determination unit.

If the difference between the first frame image and the still image is judged to be large according to the comparison, the user is notified of the judgement result and/or the radiation emission and imaging is stopped.

In this way, if it is judged that the image cannot be used for diagnosis, the user is notified that the subject S has moved from the desired position. In response to the notification, the user confirms whether or not the taken image can be used for diagnosis, and stops imaging if it cannot be used for diagnosis. This prevents the subject S from being exposed to an unnecessary radiation

If imaging is automatically stopped when it is judged that the subject S has moved from the desired position, it is possible to further prevent the subject S from being exposed to an unnecessary radiation.

If information between adjacent frame images acquired by serial imaging is important, a judgement may be made on the basis of not only the first image but a plurality of images from the first one until it becomes possible to judge information between adjacent frame images.

In this way, it is possible to accurately determine whether or not the image can be used for diagnosis even in imaging procedures where the difference between a plurality of adjacent frame images is important for diagnosis.

If it takes time for image processing etc. and the judgement is not in time for the second imaging, a judgement of the first image may be performed in parallel with the second and later imaging, as in the “positioning confirmation based on taken image” described above.

As a result, even if it takes time for image processing, second and later imagings are continuously performed without delay. At the same time, when it is determined that the image is not available for diagnosis, motion of the subject S from the desired position is notified to the user. In response to the notification, the user confirms whether or not the images being taken can be used for diagnosis and stops imaging when it cannot be used for diagnosis. This prevents the subject S from being exposed to an unnecessary radiation.

If imaging is automatically stopped when it is judged that the subject S has moved from the desired position, it is possible to further prevent the subject S from being exposed to an unnecessary radiation.

### [Body Motion Detection Method (1)]

In serial imaging, it may be difficult to distinguish on the basis of the taken images only, whether the motion of subject S is the specific body motion (that is, respiration) to be diagnosed or body motion not to be diagnosed (that is, body motion other than the specific body motion).

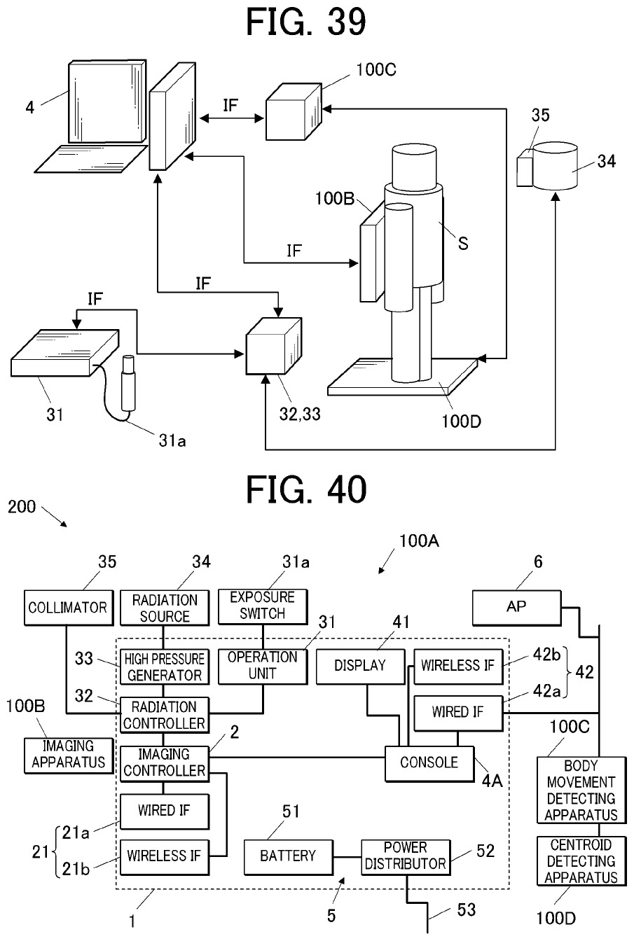

In view of such a problem, for example, as shown in FIGS. 39 and 40, the radiation imaging system may include a centroid detecting apparatus 100D which detects the center of gravity of the subject S, so that change in the position of the center of gravity may be detected during imaging as body motion other than the specific body motion.

When the subject S gets on the centroid detection apparatus 100D, the load applied to at least three points on a plane is measured (the points may be collected to measure and calculate linear pressure or surface pressure), for example, and the center of gravity can be estimated from the coordinates of the each measurement point and load value.

In addition, the body motion detecting apparatus 100C has a function to receive information on the center- of-gravity position in time series and to determine the presence or absence of body motion based on the information.

If the body motion detecting apparatus 100C determines that body motion is present, the user is notified of the determination result and/or the imaging is stopped.

Since body motion due to respiration is very small, it is possible to distinguish body motion due to movement from body motion due to respiration without attaching any special equipment to the subject S.

In addition, it can be applied to various positionings (standing or sitting).

Also, it can be introduced to existing radiation imaging systems.

As in the first embodiment, the body motion detecting apparatus 100C may not be an independent device, but the console 4 may also function as the body motion detecting apparatus 100C.

Determination may be made based on time series change in the center of gravity, for example, by differentiation with respect to time. In this way, false detections can be reduced.

[Body motion detection method (2)]

In view of the problem that, in serial imaging, it may be difficult to distinguish on the basis of the taken images only, whether the motion of subject S is the specific body motion (that is, respiration) to be diagnosed or body motion not to be diagnosed (that is, body motion other than the specific body motion), the radiation imaging system may include a pressure sensor 100E which detects the surface pressure due to the subject S's own weight, so that change in pressure may be detected during imaging as body motion other than the specific body motion.

The pressure sensor 100E may be integrated with the imaging apparatus 100B. Alternatively, a sheet-like pressure sensor 100E separate from the imaging apparatus 100B may be used by being attached to the imaging apparatus 100B as needed.

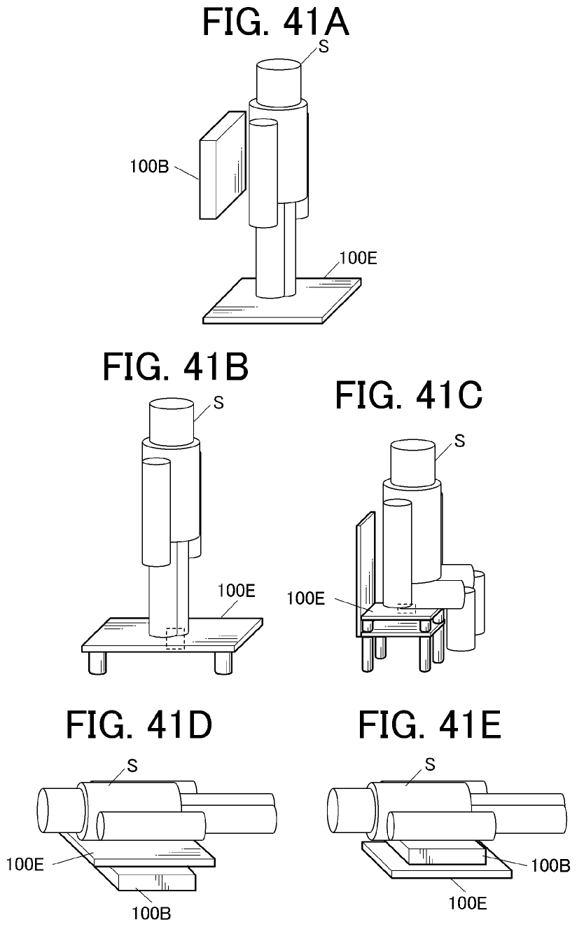

For example, as shown in FIGS. 41A to 41E, the pressure sensor 100E may be arranged on the position where the subject S stands, on the seating surface where the subject S is seated, on the bed, or the like so as to overlap the imaging apparatus 100B.

In addition, the body motion detecting apparatus 100C has a function to receive information on the center- of-gravity position in time series and to determine the presence or absence of body motion based on the information.

If the body motion detecting apparatus 100C determines that body motion is present, the user is notified of the determination result and/or the imaging is stopped.

Since body motion due to respiration is much smaller than the body motion which affects the image to be diagnosed, the pressure change due to respiration is hardly detected by the pressure sensor 100E. For this reason, the pressure change detected by the pressure sensor 100E can be regarded to be due to the body motion which affects the image to be diagnosed. Accordingly, it is possible to detect body motion due to movement distinguished from body motion due to respiration without attaching any special equipment to the subject S.

Serial imaging is different from imaging of still image in that it is possible to take dynamic images of normal body motion of the subject S. Such normal body motion includes, for example, respiration and heart beat.

In addition, when images of normal body motion of the subject S is taken, the subject S may cause body motion which is not assumed by the user or affects the diagnosis. The body motion which is not assumed by the user or affects the diagnosis includes, for example, tilt or fall of the subject S in the forward, backward, left, or right direction.

Such body motion which is not assumed by the user or affects the diagnosis is often larger than the normal body motion, and often causes pressure change larger than the normal body motion.

Therefore, if the pressure sensor 100E detects a pressure change higher than a specific pressure, it is considered that body motion which is not assumed by the user or affects the diagnosis has occurred.

Accordingly, it is possible to to detect body motion which is not assumed by the user or affects the diagnosis distinguished from the normal body motion without attaching any special equipment to the subject S.

The detection of body motion with the pressure sensor 100E can be applied to various positionings (standing or sitting).

Also, it can be introduced to existing radiation imaging systems.

The body motion detecting apparatus 100C may not be an independent device, but the console 4 may also function as the body motion detecting apparatus 100C.

### [Fixing Device]

In addition, in the detection of body motion with the above-mentioned body motion detection method, by means of a fixing device f which can fix or suppress a part of the subject S, it is possible to physically suppress flutter and motion of a joint of the subject S.

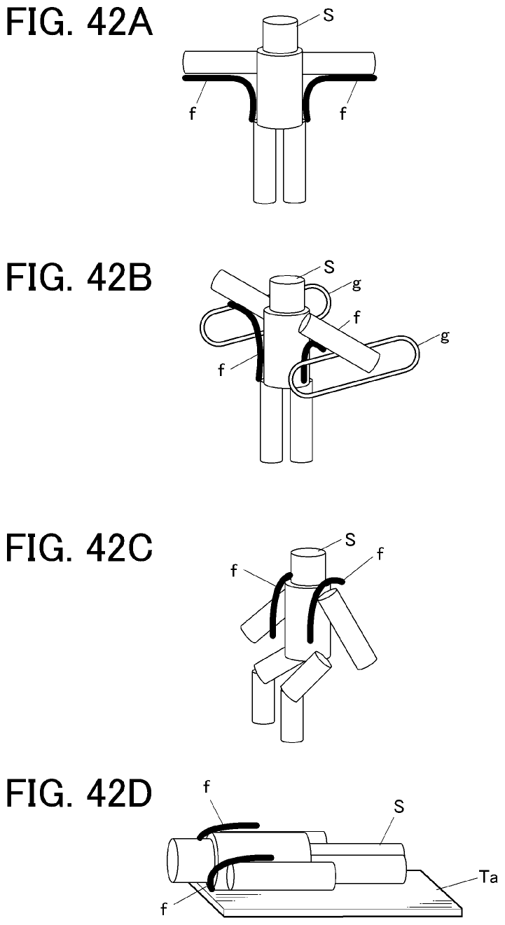

For example, as shown in FIG. 42A, the fixing device f may fix portions across a joint of the subject S, that is, trunk and arms, trunk and legs, and the like.

Further, in addition to fixing portions across the joint, the fixing device f may be incorporated in an apparatus or a device, and configured to be capable of preventing positional shift of the apparatus or the device.

For example, as shown in FIG. 42B, the fixing device f may be configured to be attachable to grip bars g in the imaging apparatus 100B.

A device other than the imaging apparatus 100B, such as a stretcher, may have the fixing device f.

The fixing device f may not fix portions across a joint, but hold the joint itself (for example, shoulders) as shown in FIGS. 42C and 42D.

In this way, the body motion itself of the subject S can be reduced.

In addition, since motion of bones is reduced, the accuracy of image processing can be improved and noise can be reduced.

Further, the fixing device f for suppressing these body motion is desirably composed of a member through which radiation passes easily. For example, the fixing device f is desirably made using a member such as a metal, through which radiation hardly passes, but a member such as a resin, through which radiation passes easily.

Alternatively, these fixing device f which suppresses body motion is desirably arranged at a position out of the region of interest observed in the imaging.

### [Body Motion Detection Method (3)]

An acceleration sensor may be used in combination with the above-described fixing device fin the imaging.

Specifically, the fixing device f includes an acceleration sensor, and a transmission unit which transmits the signal from the acceleration sensor to the body motion detecting apparatus 100C.

In addition, the body motion detecting apparatus 100C has a function to judge the presence or absence of the body motion based on the signal.

This improves the reliability of body motion detection.

### [Body Motion Detection Method (4)]

A force detecting sensor may be used in combination with the above-described fixing device fin the imaging.

Specifically, the fixing device f includes a force detecting sensor on a surface in contact with the subject S, and a transmission unit which transmits the signal from the power detecting sensor to the body motion detecting apparatus 100C.

In addition, the body motion detecting apparatus 100C has a function to judge the presence or absence of the body motion based on the signal.

Only one force detecting sensor may be used to perform detection. Alternatively, multiple force detecting sensors may be arranged continually to be used for estimation of body motion based on their correlation.

Also, the sensor may have a resolution within a specific range, or may output only a high or low signal either.

This improves the reliability of body motion detection.

### [Display Acceptable Range of Body Motion]

During radiation imaging according to the second embodiment B described above, an image of the subject S may be taken by an optical camera 43, and an acceptable region R₁ in which the body motion of the subject S is acceptable may be derived from the image and notified to the subject S or the user.

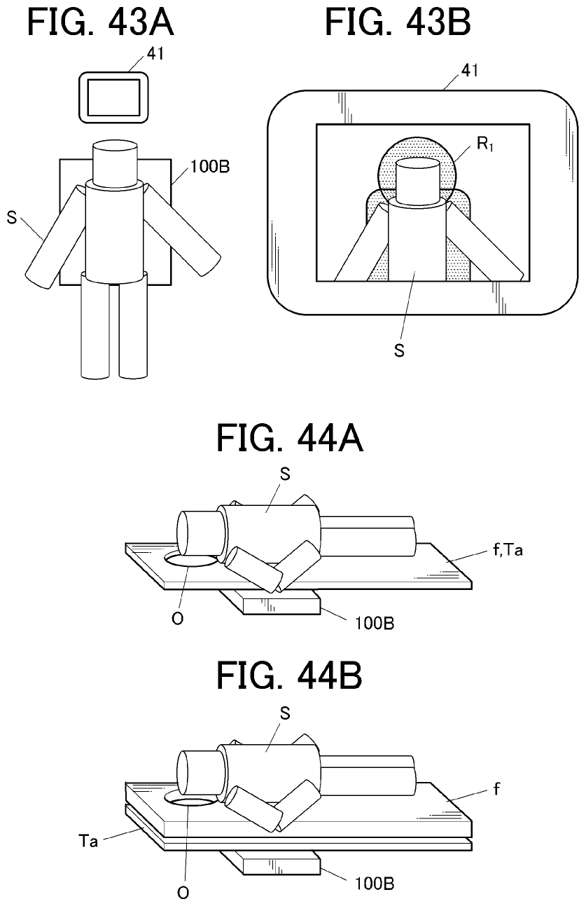

Specifically, on the basis of the image taken by an optical camera, the console 4 has a function of deriving the acceptable region R₁ in which the body motion of the subject S is acceptable. As shown in FIG. 43A, for example, a display 41 is placed at a ppace seen from the subject S. As shown in FIG. 43B, the display 41 displays the acceptable region R₁ around the subject S in the image.

In this way, since the acceptable region R₁ (range) is visually presented, the subject S can be careful not to run out of the acceptable region R₁ to adjust his/her position.

If the subject S runs out of the acceptable region R₁, imaging may be stopped.

### [Holding Unit of Subject]

In the body motion detection methods (1) and (2) described above, by means of a fixing device f which can fix or suppress a part of the subject S, it is possible to physically suppress flutter and motion of a joint of the subject S.

In particular, if the subject S is a human or an animal, whose body is not flat, the subject S may feel pain and discomfort when forced to be fixed to a flat fixing device. Therefore, it is difficult to suppress the body motion of the subject S and to keep the posture of subject S as it is for a long time.

Therefore, as shown in FIGS. 44A and 44B, for example, while the fixing device f has a flat plate shape such that the whole body of the subject S can get thereon, it has a surface with an opening O, unevenness, or a combination thereof such that at least a part of the body of the subject S enters.

In particular, if the part used in respiration such as a face is fixed in a compressed state, the subject S has difficulty in respiration and feels more pain and discomfort. As a result, it becomes more difficult to suppress body motion and to keep the posture.

Therefore, as shown in FIGS. 44A and 44B, a recess or opening O is provided at a portion facing the part used in respiration, such as the face in the fixing device f. This prevents the subject S from feeling pain and discomfort, and makes it possible to to suppress body motion and to keep the posture for a long time.

The fixing device f may be incorporated in the photographing table Ta itself as shown in FIG. 44A or may be a part removable from the photographing table Ta as shown in FIG. 44B.

In this way, the body motion itself of the subject S can be reduced.

Further, as a result of reduction in movement of bones, the accuracy of image processing can be improved and noise can be reduced.

### [Detection of Body Motion and Interruption of Imaging]

In serial imaging, it is difficult for the user to quantitatively grasp the body motion of the subject S during the imaging.

In view of such a problem, body motion of the subject S that affects the dynamic analysis may be detected from the taken dynamic image.

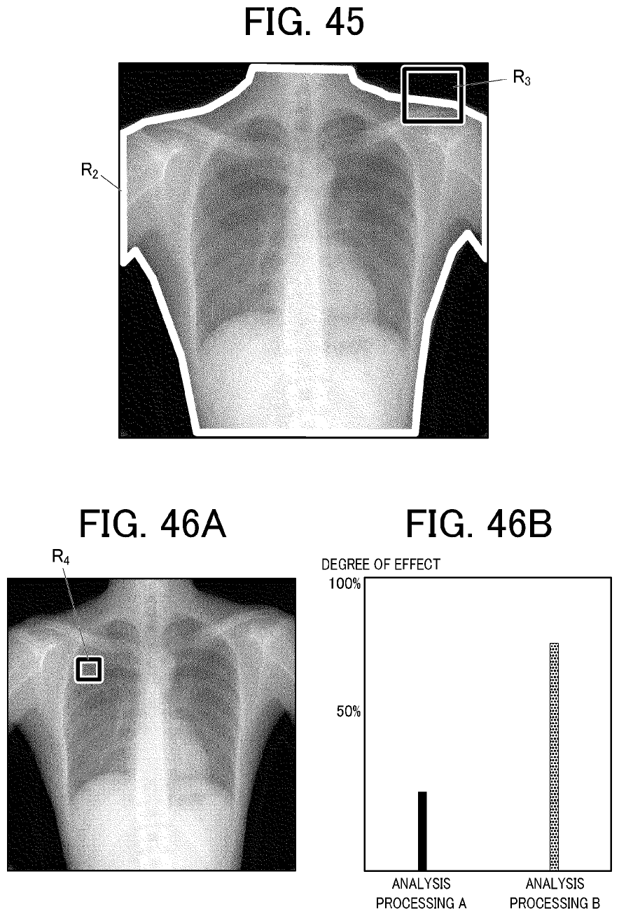

Specifically, as shown in FIG. 45, for example, the radiation imaging system is provided with an image processing apparatus (not shown) which measures a motion amount of a specific region R₃ in a body region R₂ (outline) set in a taken image, and judges whether or not the body motion has occurred on the basis of the motion amount of the specific region R₃.

For example, the body region R₂ of the subject S can be detected using a discriminant analysis method.

For example, the motion amount of the specific region R₃ can be measured using a template matching process.

If it is judged that body motion has occurred, the user is notified of the judgement result (displayed on the display 41) and imaging is stopped.

In this way, the user can determine quantitatively, not intuitively, whether or not re-imaging is required due to the body motion.

In addition, when the body motion is detected, the user may be notified of generation of body motion using the console 4 and the notification unit such as the speaker 31b, display 41, and lamp (not shown). In this way, the user can release of the exposure switch 31a etc. in response to the notification received, and interrupt the imaging

Alternatively, the imaging controller 2 may be configured to automatically interrupt the imaging in response to the detection of the body motion described above. Further, when imaging is automatically interrupted, the imaging controller 2 may be configured to notify the user that imaging is interrupted in response to the occurance of the body motion.

### [Display of Body Motion Level]

Further, for detection of the body motion of the subject S based on the dynamic image as described above, it may be determined how much the body motion of the subject S generated during imaging affects the analysis.

Specifically, a specific region R₄ is set in a body region as shown in FIG. 46A, for example. On the basis of the measured motion amount of the specific region R₄, an image processing apparatus (not shown) executes a function of calculating the degree of effects to be exhibited in the subsequent dynamic analysis.

The calculated degree of effect is displayed on the display 41, for example, in the form of a graph as shown in FIG. 46B.

Further, in consideration of the size, direction, timing, frequency, etc. of the body motion, the degree of effect can be calculated according to the type of analysis. In that case, the degree of effect on each type of analysis may be displayed as shown in FIG. 46B, for example, so that the user may be notified of the degree of effect.

For example, when amount of ventilation is analyzed as analysis processing A, the size of the lung field in each frame image can be analyzed and acquired from the taken image even if there is acceptable body motion of the subject S in left-right direction. Then, the expansion/contraction amount of the lung field can be calculated. Therefore, for example, when analysis of the expansion/contraction amount of the lung field is selected as analysis processing A of FIG. 46B, the degree of effect is calculated and displayed to be low.

On the other hand, when bone motion during respiration is analyzed, for example, the degree of effect due to the body motion is increased because the body motion not assumed by the user (that is, body motion other than the body motion due to respiration) is added to the bone motion during respiration if the whole body moves as a result of the body motion not assumed by the user. Therefore, if the bone motion analysis during respiration is selected as the analysis processing B as shown in FIG. 46B, for example, the degree of effect is calculated and displayed to be high.

In this way, the degree of effect on each analysis process to be selected due to body motion is different depending on the size, direction, timing, frequency, etc. of the body motion, but can be calculated with a corresponding appropriate method and displayed.

The user who has confirmed this display may determine to stop the imaging. In this way, the user can determine whether or not re-imaging is required due to the body motion in consideration of the effects on the subsequent dynamic analysis.

### [Determination Based on Level]

The threshold level for determining whether re-imaging is possible or imaging should be interrupted may be set in advance regarding the degree of effect.

In addition, if body motion exceeds the level set in advance, a notification unit such as the speaker 31b, display 41, or a lamp (not shown) may notify the user that body motion exceeding the level set in advance has occurred.

Alternatively, according to the level set in advance, control may be automatically made to interrupt re-imaging or imaging

Further, for detection of the body motion of the subject S based on the dynamic image as described above, the body motion of the subject S generated during imaging may be grasped not only quantitatively but also over time.

Specifically, an image processing apparatus (not shown) has a function of outputting the judgement timing if the body motion has been judged to occur.

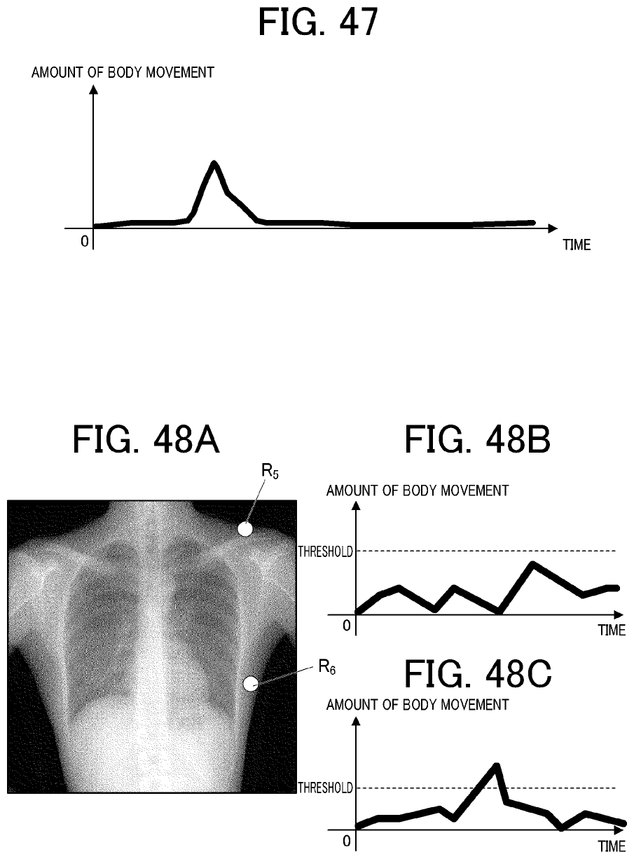

If the body motion has been judged to occur, the timing when the body motion has occurred and the amount of body motion are displayed, or imaging is stopped. The graph may be displayed as shown in FIG. 47.

The user who has confirmed this display may determine to stop the imaging

In this way, the user can determine whether or not the frame image can be used by recognizing the generation timing of the body motion, as well as determine quantitatively, not intuitively, whether or not re-imaging is required due to the body motion.

Such a degree of effect on analysis may be used not only during the imaging but also, for example, when the taken image is analyzed later. That is, the information presented to the user may be the displayed degree of effect of body motion on the analysis method to be selected or has been selected.

### [Display of Motion due to Respiration]

Further, for detection of the body motion of the subject S based on the dynamic image as described above, it may be determined whether or not the detected body motion is associated with respiration.

Specifically, an image processing apparatus (not shown) has a function of detecting body motion of the subject S which affects the dynamic analysis from the recognition result of the doby of the subject S. When the image processing apparatus (not shown) detect body motion, the display 41 of the console 4 displays the timing and amount of the body motion.

For example, specific regions R₅ and R₆ of the body region are specified as shown in FIG. 48A, and the body motion amount of each of the specific regions R₅ and R₆ is calculated to be displayed. The specific regions of the body region can be specified by, for example, template matching processing. The calculated body motion amount may be a distance or movement amount in a specific direction such as in X direction, Y direction, or body axis direction.

The calculated body motion amount may be displayed as a graph in FIGS. 48B or 48C, with the threshold (level for determination).

By setting the threshold to a value larger than the average moving amount due to body motion associated with respiration, the user can grasp whether or not body motion larger than the body motion associated with respiration has occurred.

The user who has confirmed this display can determine to stop the imaging.

In this way, the user can determine quantitatively, not intuitively, whether or not re-imaging is required due to the body motion. Further, the user can determine whether or not the image has been taken at a timing available for diagnosis by recognizing the timing of the body motion. Then, it is possible to make a diagnosis using an image taken at a timing available for diagnosis, when it is determined that there is no body motion larger than the body motion associated with respiration.

Further, it is determined whether or not the body motion is associated with respiration.

The determination may be made on the basis of the correlation of body motion amounts calculated from the respective multiple specific regions R₅ and R₆ in FIG. 48A. Alternatively, it may be made on the basis of the body motion amount of the specific region R₅ or R₆.

[Determination not based on Motion due to Respiration]

Further, for detection of the body motion of the subject S based on the dynamic image as described above, the specific region for measurement of the motion amount may be limited to be in a portion (hereinafter, immobile portion) assumed not to move with respiration in the body region.

The immobile portion may be, for example, lumbar spine, lung apex, and lung apex line.

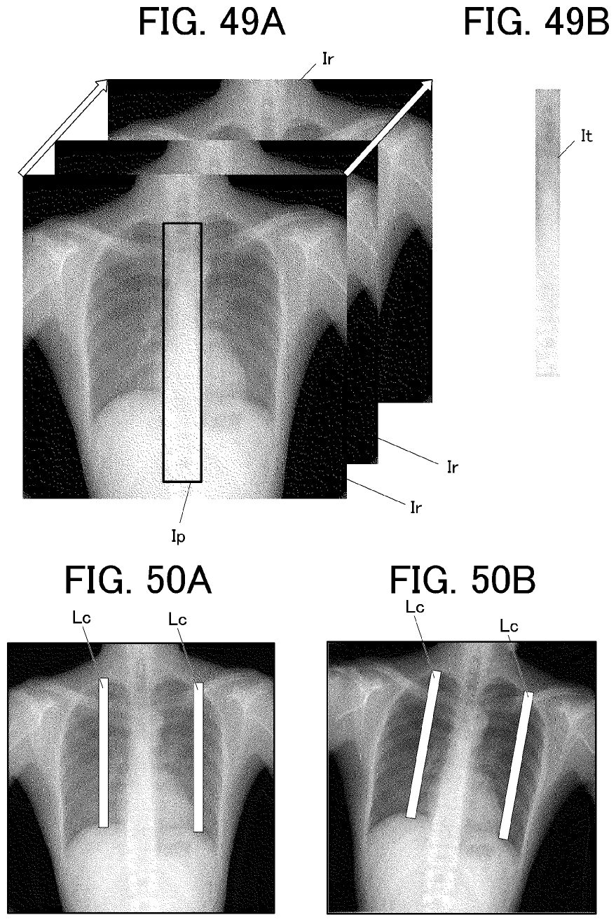

In the processing method for detecting body motion from the immobile portion, for example, the position of the immobile portion or a template image It as shown in FIG. 49B is stored for each image in advance in the console or a dedicated device. Then, as shown in FIG. 49A, a partial image Ip of the immobile portion is extracted from each image according to the position stored in advance, or the image of the immobile portion is extracted from each image based on comparison with the template image It stored in advance. Such an extraction method using a template image includes evaluation of the correlation of images, pattern matching processing, and the like.

For example, an image of the spine, which is less likely to move with respiration, is stored in advance as a template image It, of which correlation with each of the taken images is evaluated. In the pattern matching processing, the position of the spine is specified in each of the taken images. From the specified portion of the spine, the relative position (the distance from the edge of each image) of the spine in each of the images can be grasped.

When the immobile portion extracted in this way moves during the imaging, it is determined that motion which is not due to respieratioin (that is, body motion) has occurred. Further, feature quantities such as the amount of lateral movement and the amount of movement of the center of gravity may be further extracted from the extracted immobile portion. This makes it possible to grasp the degree of body motion of the immobile portion.

In such a case, if it is further determined that the feature amount exceeds a threshold set in advance, the user may be notified that the feature amount exceeds the threshold, that is, there may be an abnormality such as body motion, through sound output from the speaker 31b, display by the display 41, light emission of a lamp (not shown), and the like.

Alternatively, if it is determined that the feature amount exceeds a threshold, emission of radiation may be stopped so that the imaging is interrupted or stopped. In such a case, the user may be notified that the imaging is interrupted or stopped because the feature amount exceeds the threshold, through sound output from the speaker 31b, display by the display 41, light emission of a lamp (not shown), and the like.

Alternatively, the feature amount may be displayed as a numerical value or a graph, so that the user recognizes occurrence of body motion that causes necessity of re-imaging.

In this way, it is possible to determine whether or not body motion affects the dynamic analysis at the time of detection of the body motion. Therefore, it is easy to determine whether to stop the imaging because of the body motion.

[Body Motion Detection based on Position of Lung Field]

A specific structure (including the above-described immobile portion) which is not affected by respiration may be used in a method of detecting the body motion of the subject S generated during imaging. However, it is difficult for the user to detect and track such a structure.

In view of such a problem, the lung field region in each frame image is extracted, and as shown in FIG. 50A and FIG. 50B, for example, nearly vertical center lines Lc are drawn in the left and right lung fields. From the positional relationship between the two center lines Lc, occurrence of body motion and motion amount (parallel movement, rotation, twist, hereinafter referred to as body motion information) by body motion may be determined.

In particular, the overall motion (such as parallel movement, rotation, and twist) may be difficult to be grasped even with track of the motion of a specific portion in the image. By setting an auxiliary line such as the center line Lc in a specific portion of the image and tracking its motion as described above, the user can easily grasp such overall motion. The motion amount of the auxiliary line is calculated as the body motion amount.

The auxiliary line is, for example, a center line Lc of a specific region, a symmetry line of a region having symmetry, or the like, such that moving amount of the overall motion (such as parallel movement, rotation, and twist) may be calculated based on motion of the auxiliary line.

The center line Lc of the specific region may be, for example, a center line of a lung field, a center line of a specific organ, or a center line of a specific bone.

A symmetry line of a symmetry region may be, for example, a symmetry line of left and right lung fields, a symmetry line of left and right ribs, or the like. Since many organs are arranged symmetrically in the human body in particular, it is possible to draw a symmetry line for them and to calculate the motion thereof.

If body motion is detected during the imaging, the user may be prompted to stop imaging. Alternatively, an image may be further taken and subjected to subsequent correction processing based on the body motion information (parallel movement, rotation, and twist).

In this way, body motion information (parallel movement, rotation, and twist) can be extracted with high accuracy without detection of a specific structure which is difficult to be detected.

In addition, it becomes possible to calculate a quantitative value of the body motion information (parallel movement, rotation, and twist) from the motion amount of the auxiliary line such as the center line Lc and symmetry line.

[Body Motion Detection based on Direct Radiation Region]

From the viewpoint of processing time, it was difficult to detect a specific structure used for body motion detection during imaging. (It was difficult to prepare software and hardware that can realize real-time processing.)

In view of such a problem, area calculation of a direct radiation region Rd, where the subject S is not present as shown in FIG. 51, is performed for each frame image during serial imaging Occurrence of body motion may be determined based on the change in the region.

The direct radiation region Rd, where radiation enters the imaging apparatus 100B without passing through the subject S, is irradiated with radiation much stronger than the region where radiation enters through the subject S. Therefore, in the radiographic image Ir, the direct radiation region Rd has a much darker color than the body region R₂ in which the subject S can be seen. Therefore, when a density threshold is set between the average density of the body region R₂ and the density of the direct radiation region Rd, the area of the direct radiation region Rd can be easily calculated.

In this way, processing can be performed in real time even with a device having low computational processing capacity, since it is not necessary to detect a specific structure, but to perform a simple extraction process of a solid portion only.

[Detection based on Change in Region of Interest]

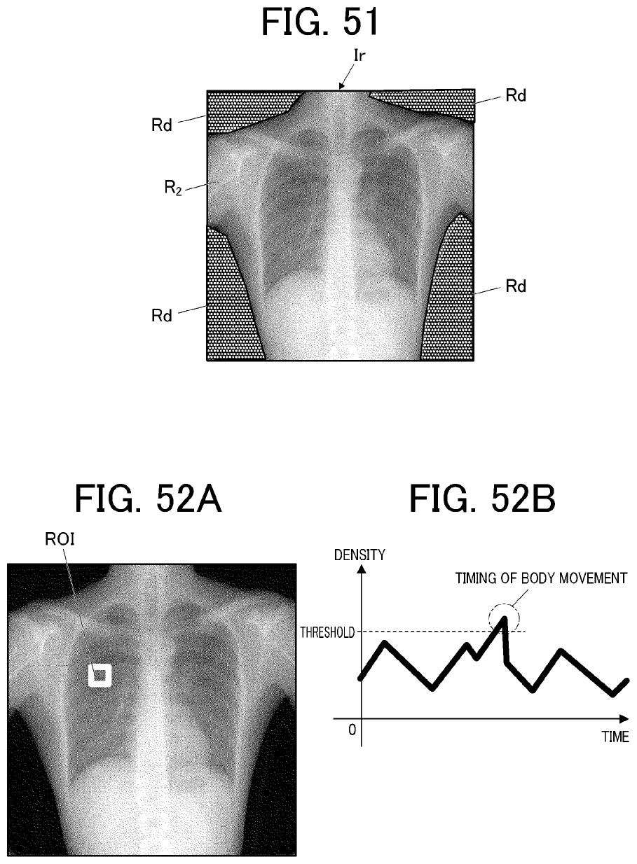

Further, in view of the problem that it was difficult to detect a specific structure used for body motion detection during imaging from the viewpoint of processing time, as shown in FIG. 52A, for example, a region of interest (ROI) may be set in the lung field, and occurrence of body motion may be detected on the basis of fluctuation of color density extracted from the region of interest (ROI) during serial imaging

Specifically, the processing unit such as the console 4 analyzes the image data input to the console 4 and the like, to acquire the density and change in the density of the image in the region of interest (ROI) and generates time-series data. The generated time series data (graph) may be displayed on the display 41 or the like so that the user can recognize the timing of body motion.

Also, it may be possible to set a threshold for the density of the image in the region of interest (ROI) or change in the density.

When it is determined that the density of the image in the region of interest (ROI) or change in the density acquired from the image data being imaging has exceeded the threshold set in advance, the processing unit such as the console 4 may notify the user, using the speaker 31b, display 41, a lamp (not shown), and the like, that the density of the image in the region of interest (ROI) or the change in the density has exceeded the threshold, that is, there may be an abnormality such as body motion.

Alternatively, if it is determined that the density of the image or change in the density has exceeded the threshold as shown in FIG. 52B, emission of radiation may be stopped so that the imaging is interrupted or stopped. In such a case, the user may be notified that the imaging is interrupted or stopped because there may be an abnormality such as body motion, through sound output from the speaker 31b, display by the display 41, light emission of a lamp (not shown), and the like.

In this way, processing can be performed in real time with less possibility of failure since it is not necessary to detect a specific structure, but to perform a simple density extraction process only.

[Detection based on Dynamic Analysis]

It is difficult for the user to determine whether or not dynamic analysis of the taken dynamic image can be performed without any problem (mainly whether or not body motion is occurring).

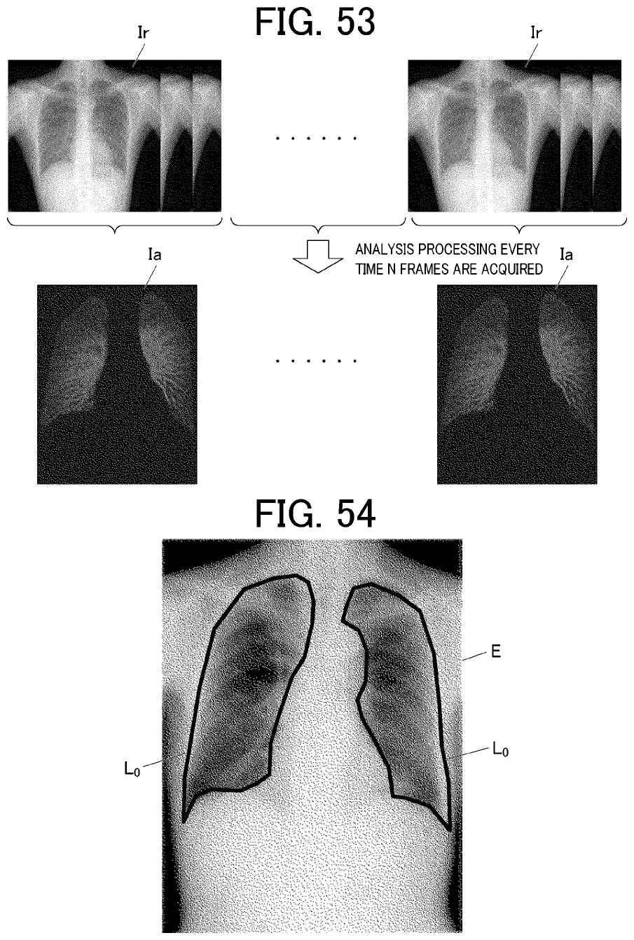

Therefore, as shown in FIG. 53, for example, dynamic analysis may be started using frame images of a predetermined number (N frames) acquired during serial imaging, and analysis results may be displayed on a display 41 or the like from the middle of the imaging

In this way, it is possible to determine whether or not dynamic analysis of the taken dynamic image can be performed without any problem from the middle of the imaging If it is determined that there is any problem, imaging is immediately interrupted. This prevents the subject S from being exposed to an unnecessary radiation.

[Detection based on Region of Interest and Imaging Region]

The serial imaging may be continued without imaging of the desired imaging target region. Then, the subject S is exposed to an unnecessary radiation.

In view of such a problem, if the desired imaging target region goes out of the imaging range during serial imaging, a message prompting the user to stop the imaging may be displayed on the display 41 or the like even if the amount of body motion is small.

Whether or not the imaging target region goes out of the imaging range may be determined depending on whether or not the outline Lo of the imaging target region intersects the edge E of the taken image, as shown in FIG. 54, for example.

In such a determination, the processing unit such as the console 4 extracts the region of interest in each image from the input image data, to make an outline Lo which follows the edge of the ROI. It is determined by calculation whether this outline Lo intersects the edge E of the taken image.

If it is further determined that the outline Lo intersects the edge E of the taken image, the user may be notified that the outline Lo of the region of interest intersects the edge E of the taken image, that is, there may be an abnormality such as body motion, through sound output from the speaker 31b, display by the display 41, light emission of a lamp (not shown), and the like.

Alternatively, if it is determined that the outline Lo of the region of interest intersects the edge E of the taken image, emission of radiation may be stopped so that the imaging is interrupted or stopped. In such a case, the user may be notified that the imaging is interrupted or stopped because the outline Lo of the region of interest intersects the edge E of the taken image, through sound output from the speaker 31b, display by the display 41, light emission of a lamp (not shown), and the like.

Alternatively, the distance between the outline Lo and the edge E of the taken image may be calculated and displayed as a numerical value or a graph, so that the user recognizes whether or not the region of interest is approaching the edge of the taken image, and, if so, the degree of approach. Alternatively, the distance between the outline Lo and the edge E of the taken image is repeatedly calculated, and when the distance is equal to or less than a specific distance, the above-mentioned warning may be issued or the imaging may be stopped

In this way, it is possible to reduce the possibility of inadvertently continuing serial imaging without imaging of the desired imaging target region.

[Detection for each Specific Region]

It was difficult to distinguish the specific body motion associated with respiration from other body motions on the basis of one kind of motion only.

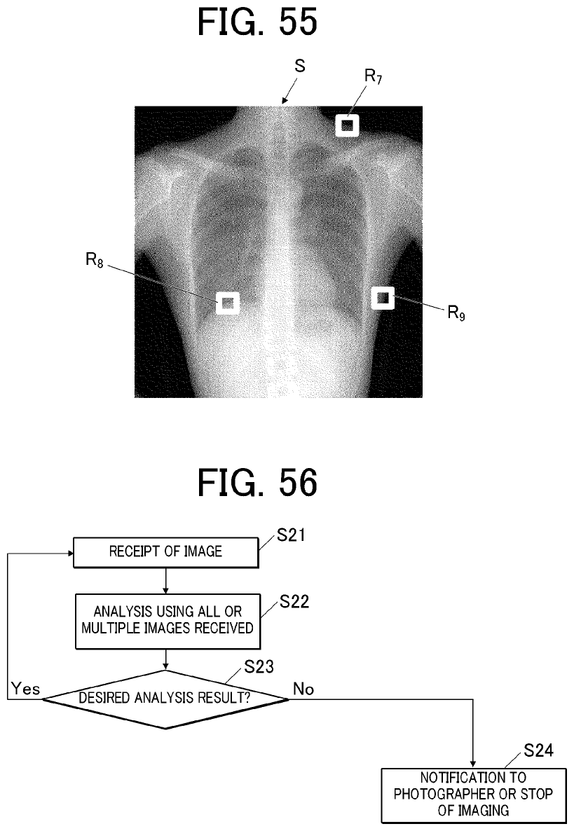

In view of such a problem, for example, as shown in FIG. 55, when the motion (quantity and direction) of the specific regions R₇ to R₉ of the subject S detected during serial imaging deviates from the basic motion of the specific region R₇ to R₉, it may be determined that them is a body motion other than a specific body motion.

The specific regions R₇ to R₉ may be, for example, a shoulder, a flank, or a diaphragm. The amount and direction of motion of these portions associated with normal respiration are within a certain range. Therefore, threshold values of the amount and direction of motion are set in advance as an acceptable range for each of the specific regions R₇ to R₉. If it is determined that the detected amount and direction of the motion of the specific regions R₇ to R₉ exceed the threshold values, it can be determined that there is a body motion.

For example, shoulders and a diaphragm basically move up and down during respiration. The maximum moving amount during a general respiration condition can be obtained by imaging and set as the amount of motion.

Specifically, the shoulder and the diaphragm are respectively set as the specific regions R₇ and R₈ for which amount of movement in the lateral direction (which is not basic motion) are each evaluated. This makes it possible to determine whether the motion the shoulder and the diaphragm during the imaging or during the image analysis after the imaging is different from the basic motion due to respiration.

Also, the flank moves basically in the lateral direction during respiration. As for the motion of the flank, the maximum moving amount during a general respiration condition can be also obtained by imaging and set as the amount of motion.

Specifically, the flank is set as the specific region R₉ for which amount of movement in the perpendicular direction (which is not basic motion) is evaluated. This makes it possible to determine whether the motion of the flank during the imaging or during the image analysis after the imaging is different from the basic motion due to respiration.

When it is determined that there is body motion, a message prompting the user to stop the imaging may be displayed on the display 41 or the like.

Also, the user may be notified that there may be caused body motion not due to respiration in the specific regions R₇ to R₉, through sound output from the speaker 31b, display by the display 41, light emission of a lamp (not shown), and the like.

Alternatively, if it is determined that the detected amount and direction of the motion of the specific regions R₇ to R₉ exceed the threshold values, emission of radiation may be stopped so that the imaging is interrupted or stopped. In such a case, the user may be notified that the imaging is interrupted or stopped because there is body motion caused in the specific regions R₇ to R₉, through sound output from the speaker 31b, display by the display 41, light emission of a lamp (not shown), and the like.

The threshold for each of the specific regions R₇ to R₉ may be set independently for the motion amount and motion direction.

The motion amount and motion direction of the specific regions R₇ to R₉ associated with the specific body motion are within a certain range. Therefore, according to the present embodiment, the body motion can be detected with high accuracy.

[Analysis during Imaging Period]

It was not possible to determine before dynamic analysis (for example, ventilation analysis) whether or not the dynamic image was desirably obtained by serial imaging.

In view of such a problem, dynamic analysis of the frame images from the imaging apparatus 100B may be started during serial imaging, such that whether or not the analysis result is desirably obtained may be determined during the imaging.

The dynamic analysis is performed with the console 4 or a dedicated device.

The console 4 or dedicated device performs, for example, the processing shown in FIG. 56. First, frame images are received (step S21), and the received frame image are analyzed (step S22). Specifically, for example, it is determined whether the difference with the data of the analysis result used as a previous frame image or a template is more than predetermined value. Here, if the analysis result is the desired result (the difference is small) (step S23; Yes), the process returns to step S21 and the frame image is continually received. On the other hand, if it is determined that the analysis result is not the desired result (Step S23; No), the user is notified of the determination or imaging is stopped.

These operations can be performed during imaging of another designated imaging period, in parallel with the imaging.

In this way, it is possible to determine whether or not desired analysis result can be obtained during imaging According to the determination result, notification may be made or imaging may be stopped, in order that the subject S is prevented from being exposed to an unnecessary radiation.

[Image Transfer during Imaging]

A dynamic image obtained in serial imaging is composed of many frame images and has a large data size, so that it takes time to transfer the data to an image server (PACS or the like) via wireless communication.

In view of such a problem, frame images output by the imaging apparatus 100B may be transferred to the image server not after completion of serial imaging, but sequentially during serial imaging.

The access point used at that time may be the same as that connected immediately before imaging

The access point communicates with the console 4 and is connected to the image server in the network.

Since console 4 does not move during serial imaging, communication with the access point connected immediately before imagine is unlikely to be interrupted. Therefore, according to the present embodiment, the dynamic image can be stably sent to the image server.

### [Display of Delayed Image]

In the case of serial imaging, it is not possible to carry out the real confirmation of the taken image only after termination of the imaging. Therefore, if re-imaging is needed as a result of the confirmation, subject S is exposed to an extra radiation corresponding to serial imaging of one time.

In addition, if a real-time frame image is displayed, for example, the user cannot check the image taken during a period when the user is directly viewing the subject S. As the user may start imaging operation while directly viewing the subject S especially in the early stages of imaging, the user cannot check the image immediately after the start of imaging

Also, while the body motion that appears on the surface of the subject S can be checked by imaging during direct observation of the subject S, internal body motion that does not appear on the surface of the subject S cannot be checked without check of the taken image. However, the state (suffering and the like) of the subject S can be easily confirmed by direct observation of the subject S.

In view of such a problem, during serial imaging, the display 41 or the like of the system main body 100A may display not a real-time frame image currently being taken but a frame image taken several seconds ago (i.e., with a time lag).

Thus, as the display 41 or the like displays not a real-time frame image but a frame image taken a little while ago, the user can start the imaging operation while directly observing the subject S at the beginning of the imaging By turning his eyes to the display 41 or the like after that, the user can take images during check thereof, which is more important than direct observation for diagnosis, over the entire imaging period.

Further, according to the the present invention including slightly delayed display of the taken images, the user can start imaging after confirming the state of the subject S (whether or not the subject S is suffering, and the like) at the start of the imaging Further, after that, the user confirms the taken images displayed with delay and continues imaging if there is no abnormality, so as to take images during check thereof over the entire imaging period. On the other hand, if there is an abnormality such as body motion or lack of picture, the user can determine to stop imaging in the middle of the imaging Therefore, even if re-imaging is needed, the exposure dose can be reduced due to the stop of imaging.

Alternatively, a dedicated device having a function of displaying this delay may be provided.

The frame image may be displayed not only with delay, but may be subjected to image processing such as adding some kind of mark (annotation, stamp, etc.) in the pre-display period. Specifically, for example, a stamp such as “L” or “R” indicating the direction of taking the image may be added in this period.

### [Confirmation Mark]

Further, as described above, if a frame image taken a few seconds before is displayed on the display 41 or the like instead of the frame image currently being taken, a mark may be added to some of the frame images.

Specifically, the console 4 or dedicated device has a function of adding some kind of mark (annotation, stamp, etc.) to multiple frame images (not in real time) to be displayed.

Such a mark may be added to the frame image designated by the user or to the frame images before and after the designated frame image.

Alternatively, the mark may be added to the frame image from which the console 4 or the like determines that the threshold value of the above-mentioned body motion amount and the image density have been exceeded, or to the frame images before and after thereof.

In addition, only the frame images with mark may be collected into another folder, or transferred to an external image server.

When a frame image taken several seconds ago is displayed, it may take time to search for and/or display the frame image with abnormality. However, according to the above function of adding the mark to frame images in addition to displaying the delay, the user can not only search for an image of high interest immediately after imaging but determine whether or not re-imaging is needed and select images to be diagnosed immediately.

### [Cutout of Image]

Further, if a mark is added to the frame images as described above, the frame image with mark may be automatically cut out and displayed after imaging.

Specifically, the console 4 or a dedicated device has a function of cutting out the marked images so as to display the marked image only.

The image to be cut out may be a still image or a part of a moving image.

In addition, only the frame images with mark may be collected into another folder, or transferred to an external image server.

Further, if a mark is added to the frame images as described above, it may take time to display and/or cut out the frame image with abnormality. However, according to the above function, the frame image with abnormality can be found immediately even during imaging. Further, it is possible to reduce the user's time and labor of removing (trimming) unnecessary frame images.

[Mark according to Difference in Images]

Further, as described above, if a frame image taken a few seconds before is displayed on the display 41 or the like instead of the frame image currently being taken, a mark may be added to successive frame images which are largely different from each other.

Specifically, the console 4 or dedicated device has a function of calculating the difference between one acquired frame image and the previously acquired frame image and judging whether or not the difference exceeds a predetermined threshold, and a function of adding some kind of mark (annotation, stamp, etc.) to the frame image(s) when it is judged that the difference exceeds the threshold.

The mark may be added not only to the largely different frame images, but also to several frame images before and after them.

When a frame image taken several seconds ago is displayed, it may take time to search for and/or display the frame image with abnormality. However, according to the above configuration, the frame images with abnormality can be found immediately with high accuracy during imaging Also, the user can not only search for an image of high interest immediately after imaging but determine whether or not re-imaging is needed and select images to be diagnosed immediately.

### [Adjustment of Emission Field]

If the field to which radiation is emitted shifts due to motion of the subject S during serial imaging, the dose of radiation between frame images may be uneven or a frame image having no image may be present. Thus, there is a problem that analysis results may be affected in the subsequent processing, and the like.

In view of such a problem, the collimator of the radiation imaging system including the body motion detecting apparatus 100C according to the second embodiment may include an emission field moving device which moves the emission field in the direction orthogonal to the optical axis Ao, not only the function to narrow or expand the emission depending on the information from the body motion detecting apparatus 100C.

A camera, a pressure sensor, etc. may be used to detect the body motion.

In this way, even if the subject S unexpectedly moves in the serial imaging, the emission field may be moved, so that it is possible to reduce the uneven dose among the frame images, the increase of the noise in the images, and the like.

### [Volume Adjustment]

In imaging using the movable vehicle 100A in a general ward, such as the bedside of the subject S, since the radiation source 34 is close to the subject S, a buzzing sound for radiation emission notification may be heard loud to the subject S. Since many subjects S do not routinely experience radiation imaging, they are likely to be sensitive to the sound from the radiation source 34. Also, in general wards, other nearby patients may be afraid of invisible radiation In particular, a long buzzing sound is generated in serial imaging of a body motion such as respiration. This may cause the subject S and other patients to feel more anxiety. However, the buzzing sound is information necessary to notify the user that the radiation is being emitted.

In view of such a problem, the volume of the buzzing sound for radiation emission notification may be configured to be adjustable.

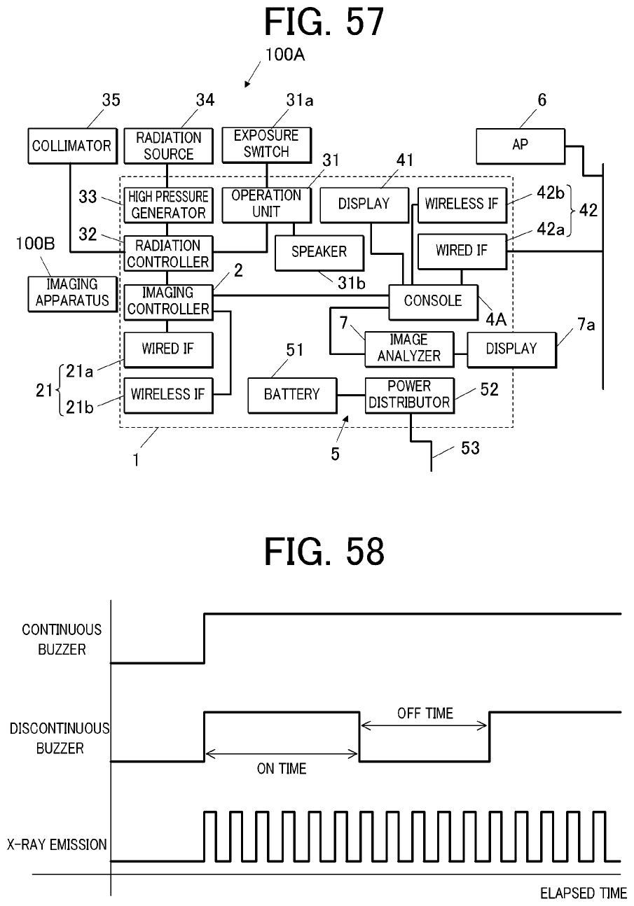

Specifically, as shown in FIG. 57, for example, a speaker 31b which generates a buzzing sound is connected to the operation unit 31. The operation unit 31 may include, for example, a hardware such as a dial for adjusting the volume, or may use a touch panel or a button used for setting the imaging conditions also for adjusting the volume. This makes it possible to eliminate the anxieties given to the subject S and people around the subject S.

The volume may be adjusted while the buzzing sound. In this way, it becomes easy to adjust the volume to a level suitable for the subject S and the user.

### [Use of Intermittent Sound]

The buzzing sound for notifying radiation is made only while radiation is being emitted. However, in serial imaging where emission of radiation is repeated in pulses, the buzzing sound seems to be continuously made because the time between one emission of radiation and the next one is too short. A buzzing sound that continues for a long time may cause the subject S to feel great anxieties, and not to breathe stably.

In view of such a problem, the buzzing sound in serial imaging may be an intermittent sound, for example, which is turned on and off alternately repeatedly at a cycle longer than the radiation emission cycle as shown in FIG. 58.

Preferably, the time to turn off the buzzing sound is not too long such that the user should not misunderstand that the emission has finished.

The time to turn on or off the buzzing sound may be adjusted depending on the preference of the user.

In this way, in serial imaging, which takes a long time, it is possible to eliminate the anxieties given to the subject S and the people around the subject S without losing the buzzing sound notification function.

[Notification not with Sound]

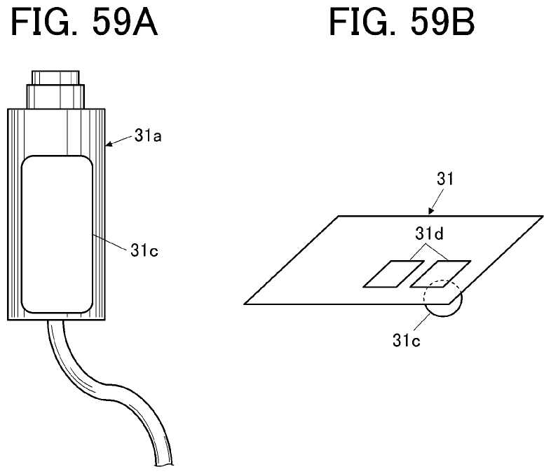

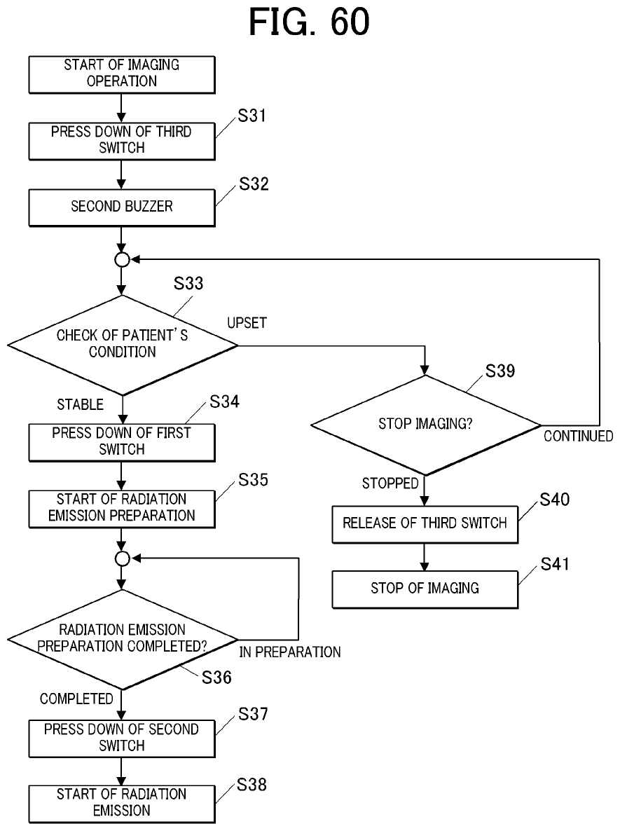

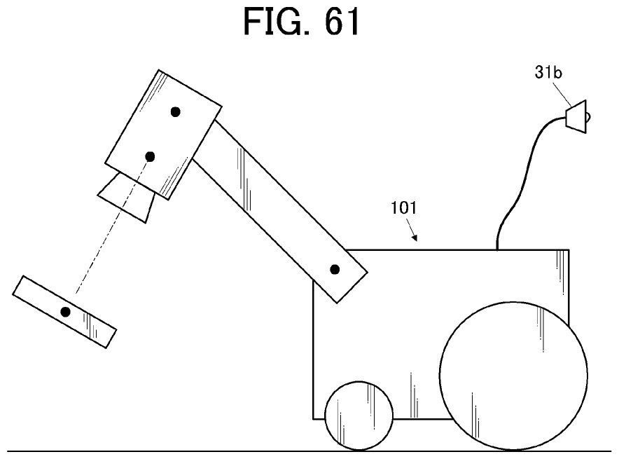

In view of the problem that the buzzing sound for radiation emission notification which sounds loud may cause the subject S and other patients to feel more anxiety, as shown in FIG. 59A for example, a switch used for start of radiation emission such as the exposure switch 31a may have a vibration source 31c which generates, during radiation emission, vibration that can be felt by the user.

When the operation unit 31 includes the button 31d as illustrated in FIG. 59B, for example, the vibration source 31c may be provided at the back of the button 31d.

The switch to start and continue the radiation emission needs to be pressed down throughout the radiation. That is, the user is in contact with the switch throughout the radiation emission. Therefore, according to the present example, it becomes possible for the user to recognize information during radiation emission based on the vibration of switch which the user is always in contact with, thereby to recognize information on the radiation emission without relying on auditory information such as a buzzing sound or visual information such as an LED or display. Since the subject S and the people around the subject S who are not involved in the imaging do not receive information on vibration, they are less likely to notice that radiation emission is being performed. This removes their nervousness and anxieties about the imaging.

Bone conduction may be used to inform the user of radiation emission information using a device connected with wire or wirelessly.

In this way, only the user can recognize the radiation emission information as a sound which does not disturb normal air conduction sounds vibrating the tympanic membrane through the air, such as communication with the subject S, voice of the subject S during imaging, and the sound of devices.

### [Use of Preceding Sound]

In serial imaging, imaging of the lung field when during respiration in a resting state may be desired. However, since radiation imaging is an unusual event, the subject S is likely to become tense and mentally unstable.

The user calms the subject S to some extent by communicating with the subject S before imaging. However, the buzzing sound to notify the user of the emission is made when radiation imaging is generally started, and continues as long as the emission is being performed. In addition, when radiation is emitted, mechanical sound is generated from the system main body 100A. The subject S recognizes that radiation imaging has been started or is in progress by listening to these sounds, and may fall into nervousness again and may not be able to breathe in a resting state.

In view of such a problem, the exposure switch 31a may have a third switch, in addition to the first switch and the second switch respectively used to start preparing radiation emission and to start emitting radiation For example, as shown in FIG. 60, when the third switch is pressed (step S31) before the first switch is pressed down (step S34), a buzzing sound (second buzzing sound) may be made that is similar to the first buzzing sound made in response to pressing of the first or second switch (step S32).

The second buzzing sound has a tone different from the first buzzing sound for radiation emission notification so that the difference from the first buzzing sound can be determined.

As a result, the subject S may be temporarily nervous by receiving information similar to that during imaging, but continuation of this state allows the subject S to be calm again. The user can press the first switch and the second switch to perform imaging as the subject S gets used to the state and calm.

An example using the third switch is described above, but the third switch may not be used in the present invention. For example, a device having only the first and second switches performs the same operation (makes the preceding buzzing sound (second buzzing sound)) as that when the third switch is pressed in a predetermined period after the first switch is pressed down. In such a case, if a predetermined period of time has elapsed with the first switch is being pressed down, the same operation as that when the third switch is pressed down may be performed.

Such control makes it possible to perform imaging after the subject S gets used to the buzzing sound made in advance even with a device having only the first and second switches, not with the device having the third switch in addition to the first and second switches as described above.

Also, the sound generated in response to operation of the third switch may not be the same as the buzzing sound indicating that radiation is now emitted, but sound (volume, tone) which makes the subject S unable to hear the equipment preparation sound or the buzzing sound for radiation emission notification to the user. In this way, environmental changes on the subject S can be reduced.

Also, music may be used instead of the buzzing sound of a single sound. In this way, the subject S can be made calmer.

Alternatively, the subject S may be made to get used to a situation in which a loud sound is heard that is louder than the buzzing sound for radiation emission notification, and then the imaging may be started with less the volume. In this way, as the volume is reduced, the subject S has less feeling of pressure and the user can identify that radiation is being applied.

Alternatively, the user may be notified that radiation is being emitted not based on a sound. In this way, environmental changes on the subject S can be reduced.

Further, without using the third switch, the imaging may be started in response to transmission of the information about pressing of the first switch to the radiation source 34 not immediately after pressing the first switch, but after a predetermined time has passed. In this way, it is possible to improve operability by limiting the time for waiting for the subject S to be in the resting state.

In addition, another measuring device may be used to determine whether the subject S is in a stable state. For example, a device which measures heart rate may be used to check the value of heart rate, or a device which measures respiration may be used to check the amount of inspiratory and/or respiratory capacity. In addition, information from these measuring devices may be taken into the system via IF, to be used for automatic determination whether or not the subject S is in a stable state, and imaging may be performed. In this case, if it is not determined that the subject S is in a stable state even after a predetermined time has elapsed, the user may be notified of the determination as a system error and the imaging may be temporarily stopped. As a result, the stable state of the subject S can be determined with scientific grounds from measurable physical state, less determination by the user is required, and operability improves.

In order to make the subject S hear the sound generated in response to operation of the third switch, devices such as headphones or earphones may be used so that the subject S is more likely to hear the sound than other sounds. In this case, since the subject S cannot hear instructions from the user, the user may transmit the instructions to the subject S through a microphone. In addition, so as not to acquire extra visual information as well as auditory information, the subject S may wear a VR to be shown calming images. In this case, external information on the subject S can be limited, and environmental changes can be reduced.

The image to be displayed may be one that are not related to the imaging at all (for example, a natural scene which makes a person relax). In this case, the subject S may forget the state of radiation imaging and be in a mentally comfortable state. The image to be displayed may be an image of the subject S. In this case, the subject S may understand the imaging situation, and may cooperate with the imaging

### [Use of Distant Sound]

In imaging using the movable vehicle 100A at the bedside of the subject S in a general ward or the like, the buzzing sound for radiation generation notification may cause the subject S to be surprised, nervous, or uncomfortable.

In view of such a problem, the speaker 31b which makes the buzzing sound may not be included in the movable vehicle 100A, but may be put away from the movable vehicle main body 101 as shown in FIG. 61, for example.

The speaker 31b may be usually attached to the movable vehicle main body 101, but may be removed as needed.

The speaker 31b that makes the buzzing sound may be connected to the movable vehicle main body 101 with wire or wirelessly.

In this way, the speaker may be arranged away from the subject S, and the anxiety of the subject S due to the buzzing sound during imaging can be reduced.

Also, when the user operates the the main body of the movable vehicle 101 remotely, the user can confirm the buzzing sound nearby by bringing the speaker 31b close to the user, compared with the case where the speaker 31b is in the movable vehicle main body 101.

A speaker may be incorporated in the exposure switch 31a and a buzzing sound may be made by the exposure switch 31a. In this case, since the user always holds the exposure switch 31a during radiation emission, the sound source is located close to the user and far from the subject S. The user does not have to take the sound source separately, but only executes the operation procedure as usual.

Also, the orientation of the speaker may be changed. In this case, change of the sound output direction can reduce the volume of the sound that reaches the subject S.

The optical camera 43 may be attached to the collimator, and the subject S recorded by the optical camera 43 may be displayed on the display 41 which can display an image and is removably arranged on the movable vehicle main body 101, together with the exposure switch 31a and the speaker. In this way, even if the user moves to a position where the subject S cannot be seen to avoid being exposed to further radiation during the radiation emission, the user can grasp the state of the subject S in real time through the image.

### [Radiation Emission Instruction Switch]

The exposure switch 3 la (irradiation emission instruction switch) is a two-step switch of a push-in type and is pressed using the thumb only. Preparation of radiation is started by the first-step pressing, and radiation is actually started by the second-step pressing. Since this exposure switch 31a requires a certain amount of power to be pressed down, the user can obtain sufficient operational feeling. However, this in turn makes it difficult to keep the exposure switch 31a being pressed down during a relatively long time (about 20 seconds) in the serial imaging.

In view of such a problem, the switch for preparing radiation emission (first step) and the switch for starting radiation emission (second step) may be provided as separate buttons.

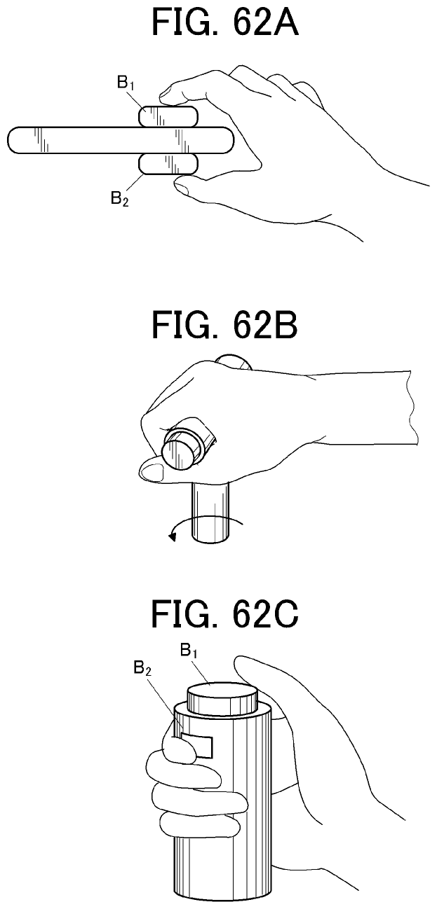

Specifically, the first button B₁ may be provided on one surface of the main body of the switch, and the second button B₂ may be provided on the other surface as shown in FIGS. 62A and 62C, for example, or at least one of the first step operation and the second step operation may be rotationally performed as shown in FIG. 62B.

In this way, operation can be made using portions other than the thumb or by putting weight, the load on the thumb may be reduced while a sufficient click feeling or operation feeling is sufficiently maintained

### [Display of Remaining Imaging Time]

Unlike still imaging, serial imaging does not finish in a moment. Therefore, the subject S may be fear and cautious about imaging, and there is a problem that imaging can not be desirably performed. For example, when subject S feels fear or cautious in imaging of lung respiration, the respiratory state may change from that in a resting state, making it difficult to image a desired normal respiration.

In view of such a problem, the subject S may be notified of the remaining imaging time during the imaging

Specifically, for example, the display 41 (see FIG. 30) showing the remaining imaging time in seconds may provided at a place which the subject S can see.

In this way, the subject S can be subject to the imaging at ease, appropriately informed of how many seconds are remaining until the imaging is completed.

Further, as the subject S can be subject to the imaging at ease, imaging in a normal respiration state can be performed, for example, which is not possible when the subject S is nervous.

[Protection from Radiation]

When multiple subjects S each take an examination using radiation imaging in one room, radiation from the radiation emitter 103 is emitted not only to the subject S to be the target of imaging but to the surrounding subjects S. There was a problem that the surrounding subjects S are exposed to an unnecessary radiation emitted from the radiation emitter 103. In particular, in the case of the serial imaging where the imaging period is long, exposure to the surrounding subjects S may be considered as a problem.

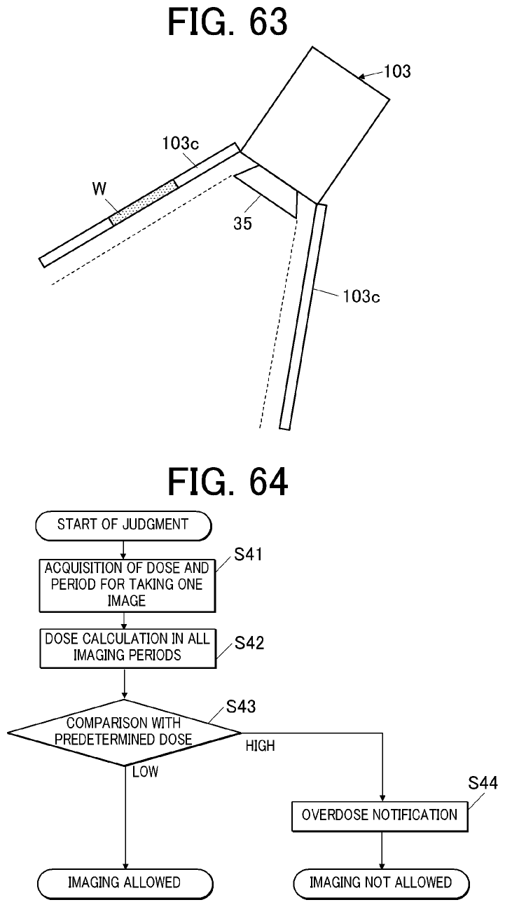

In view of such a problem, for example, as shown in FIG. 63, a shielding wall 103c opening in the radiation direction of the radiation X may be provided around the collimator 35.

The shielding wall 103c may be made of lead-containing glass or the like, for example, so as to prevent the radiation from penetrating.

For looking the inside, the shielding wall 103c may have a window W, or may be made of a transparent or translucent material.

As a result, radiation emission in unintended directions can be suppressed so that the surrounding subjects S are not exposed to an unnecessary radiation even in the same room as the subject S to be the target of imaging.

It is also possible to reduce the exposure of subjects, doctors, family members, etc. who are not in the same room (who are in a room separated with a wall).

### [Grasp of Total Radiation Dose]

When multiple subjects S each take an examination using radiation imaging in one room, radiation from the radiation emitter 103 is emitted not only to the subject S to be the target of imaging but to the surrounding subjects S. There is a problem that the surrounding subjects S are exposed to an unnecessary radiation emitted from the radiation emitter 103. In particular, there is a serious problem in the case where an unintentionally high dose of radiation is emitted for imaging due to the user's incorrect input of imaging conditions.

In view of such a problem, for example, as shown in FIG. 64, not only the radiation dose for taking one image but the total radiation dose in all imaging periods may be calculated (step S42), if the total radiation dose is more than a predetermined value (step S43: HIGH), the user may be notified of the result (step S44) or the radiation emission may be stopped.

The dose may be input and set for each imaging procedure. When an order for imaging technique is input from RIS or HIS, when the user selects the imaging technique among them, or when the user sets imaging technique, the dose associated with the imaging procedure may be set.

In this way, it is possible to prevent imaging with a high total dose of radiation throughout the imaging period, contrary to the user's intention. Then, it is possible to prevent unnecessary exposure to the subject S or the surrounding subjects S, medical workers, and family members.

[Protection with Curtains]

In view of the problem that, when multiple subjects S each take an examination using radiation imaging (in particular, serial imaging) in one room, radiation from the radiation emitter 103 is emitted not only to the subject S to be the target of imaging but to the surrounding subjects S, a material that hardly transmits radiation may be used as a curtain for visually isolating the subject S.

Specifically, the transmission of radiation may be suppressed by metal fibers mixed in the fabric of the curtain or using metal foil.

As a result, the surrounding subjects S are not exposed to an unnecessary radiation even in the same room as the subject S to be the target of imaging.

It is also possible to reduce the exposure of subjects, doctors, family members, etc. who are not in the same room (who are in a room separated with a wall).

### [Detection Using Marker]

In serial imaging, the user wants to take images of only the desired motion of the subject S (for example, respiration during imaging of lung). However, the subject S may take not only the desired motion but undesired motion (for example, a body motion of moving the body vertically, horizontally, and backward or forward, etc.). If the user notices such undesired motion and stop the imaging at that moment, the subject S will not be unnecessarily exposed to radiation. However, if the user does not notice the undesired motion, imaging is continued to the end only to take unavailable serial images, and there is a risk that the subject S is exposed to unnecessary radiation.

In addition, if the user checks the preview and notices the undesired motion (body motion) after the imaging is completed, the time of imaging and checking the preview is also a waste of time. Such a waste of time is a problem particularly in the case where the shorter imaging time is more preferred, such as in an ICU.

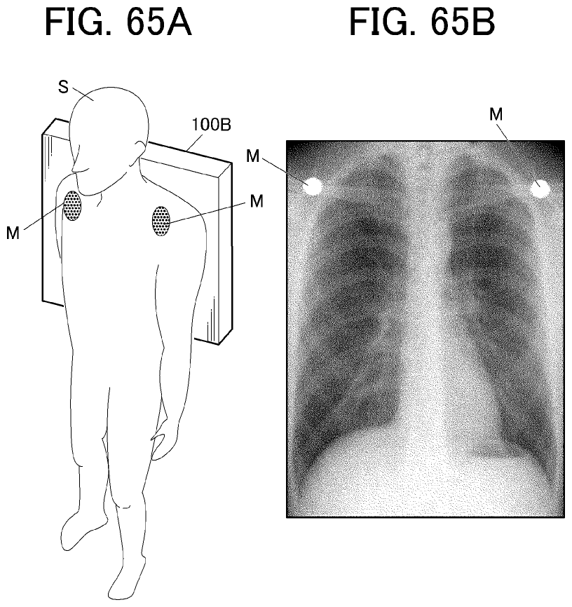

In view of such a problem, as shown in FIG. 65A, for example, a marker M having a radiation transmittance different from that of the surroundings may be attached to the surface of the subject S.

Here, “the radiation transmittance different from that of the surroundings” means that, when the subject S is a human body, the transmittance is different from the air around the marker M or the skin or body of the human body.

The marker M is preferably attached to the subject S at a portion which do not move due to the desired motion or which moves in a known direction due to the desired motion. Specifically, for example, in imaging of a lung, it is desirably attached to a body surface of a spine or a scapula.

Then, the motion of the marker M is evaluated among frame images (each shown in FIG. 65B) successively obtained by serial imaging of the subject S. It is determined whether or not the undesired motion, other than the motion that the user desires to take images of, exceeds a threshold.

If the threshold is exceeded, the user receives a warning or imaging is stopped.

As a result, it is possible to detect the presence or absence of the undesired motion of the subject S according to the motion of the marker M.

In addition, as the user receives a warning or imaging is stopped when the undesired motion is detected, the subject S is prevented from being exposed to an unnecessary radiation.

The determination may be made not based on whether or not the marker M is moving, but based on the direction in which the marker M is moving.

In such a case, the determination may be made not only based on the direction in which the marker M is moving, but also on the basis of the timing when the marker M moves.

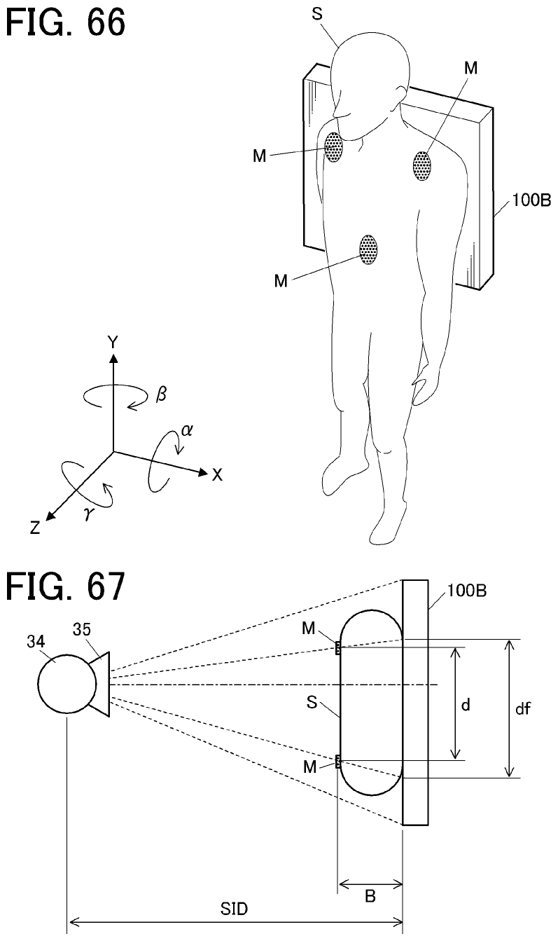

Also, as shown in FIG. 66, for example, three or more markers M may be attached to detect motion. In this way, six-dimensional motions of X, Y, Z, α, β, and y can be calculated.

In order to further improve the robustness of calculation, the number of the markers M is desirably four or more, and their motions are used for calculation. Further, the number of the markers M is more desirably six, which is the same as the number of measurement dimensions, and their motions are used for calculation.

This makes it possible to grasp the movement of subject S in multiple dimensions. Then, the subject S can be suitably notified of the direction of undesired motion and can be urged to suppress motion in the undesirable direction.

[Measurement of Distance using Marker]

The SID needs to be adjusted for radiation imaging However, the radiation emitter 103 which determines the focal point of radiation and the imaging apparatus 100B are changed depending on the imaging technique, imaging conditions, and the condition of the subject S. Therefore, it was difficult to grasp the SID and adjust it to a predetermined value.

In view of such a problem, as shown in FIG. 67, for example, the markers M may be attached to multiple places of the subject S, and SID may be calculated based on the distance d between the actual markers M, the distance df between the markers M in the image, the body thickness B of the subject S, and the like.

The distance d between the actual markers M may be measured in advance, or the markers M may be attached with a predetermined distance between them.

Further, the distance df between the markers M is calculated from the radiographic image.

The body thickness B of the subject S may be an estimated value or a measurement value with another optical measurement device or the like.

The distance d between the actual markers M, the distance df between the markers M in the image, the body thickness B, and the SID satisfy the following relation (11).

(SID-B): d=SID: df (11)

From the relation (11), SID can be calculated as in the following equation (12).

SID=df·B/(df−d) (12)

In this way, the user can grasp the SID as a numerical value, and can easily adjust it.

According to the information including the age and gender of the subject S, the body thickness B may be automatically set to be a standard value for the age and gender when the RIS or HIS sends the imaging order and may be modified by the user if necessary. Alternatively, an estimated value calculated from the abdominal circumference, chest circumference, and the like may be automatically input.

Such easy input of the body thickness information reduces the work load.

If the user fails to input body thickness information, for example, an incorrect SID based on the remaining body thickness information of the previous subject S is displayed. Therefore, adjustment to an incorrect SID can be prevented.

Further, in order to reduce the total exposure dose of the subject S, it is desirable to emit radiation for SID adjustment at a weaker intensity than radiation emitted for radiographic image acquisition used for diagnosis and the like. For this reason, the SID can be adjusted on the basis of radiation imaging for calculating the SID taken prior to radiation imaging for diagnosis and the like. For that purpose, the image from which the distance between the markers M can be calculated may be taken with a radiation emission intensity weaker than that in radiation imaging for diagnosis and the like.

As a result, the subject S can be exposed to less radiation in total.

The SID may be calculated from the radiographic image used for diagnosis or the like by the method according to the present invention, and may be displayed to the user or sent to the image server as information associated with the image.

Even if the SID is measured and adjusted before imaging, the motion of the subject S immediately before or during the imaging may result in change of the SID. Therefore, the SID during the imaging is important to be grasped but may not be exactly the same as the SID adjusted in advance. However, as described above, it is possible to know the SID at the time when the image used for diagnosis has been taken.

[Detection of Body Motion with Optical Camera]

In addition, in view of the problem that, if the user does not notice the undesired motion, imaging is continued to the end only to take unavailable serial images and there is a risk that the subject S is exposed to unnecessary radiation, and that, if the user checks the preview and notices the undesired motion after the imaging is completed, the time of imaging and checking the preview is also a waste of time, the optical camera 43 (see FIG. 11) may be placed at a portion where the subject S can be captured on the radiation emitter 103 etc. such that whether or not the subject S is moving can be judged on the basis of the optical image Io of the subject S.

Whether or not there is a motion may be determined from image processing of the entire taken image. Alternatively, it may be determined from image processing of the portion of the subject S extracted by the image processing. Alternatively, it may be determined from the motion of a specific region extracted depending on the imaging procedure.

The judgement of motion according to the taken image may be made based on the difference from the initially taken image, the difference between adjacent images, or both of them.

Then, in response to the judgement result, a warning is given to the user or the imaging is stopped.

As a result, it is possible to detect the presence or absence of the undesired motion of the subject S.

In addition, as the user receives a warning or imaging is stopped when the undesired motion is detected, the subject S is prevented from being exposed to an unnecessary radiation.

The optical camera 43 may be placed so as to take an image of the subject S not only from the front but also from the side.

[Detection based on Sensor]

There is a problem that an error occurs in the SID or the like due to an unintended movement of the radiation emitter 103 during serial imaging

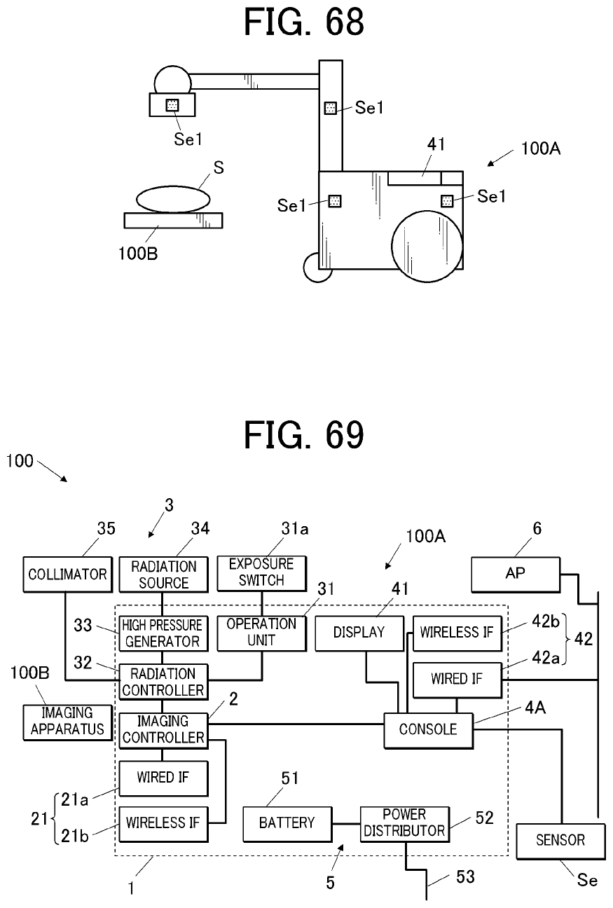

In view of such a problem, as shown in FIGS. 68 and 69, for example, the system main body 100A may include a sensor Se1 which detects the motion of the radiation emitter 103 to determine the presence or absence of positional variation of the radiation emitter 103 during serial imaging based on the output of the sensor Se1.

The sensor Se1 is, for example, an acceleration sensor, a gyro sensor, a geomagnetic sensor, a strain gauge sensor, or the like.

For example, the controller or the like of the console 4A determines the positional variation.

The positional variation may be determined on the basis of, for example, whether or not the individual output values of the sensor Se 1, the average of the output values in a certain period, the accumulation of the output values in a certain period, or the like exceeds a predetermined threshold.

The user may change the setting of the threshold according to the image accuracy or the like desirably obtained by the imaging.

In addition, on the output values, average, or accumulation may be performed processing using a filter such as a low pass filter, which realizes stable image processing.

Then, in response to the determination that the output values, average, or accumulation exceeds the threshold, a warning is given to the user or the imaging is stopped.

As a result, as the user receives a warning or imaging is stopped when the measured positional variation of the radiation emitter 103 exceeds the threshold (acceptable value), the subject S can be prevented from being exposed to an unnecessary radiation by further imaging.

The determination of whether the subject S or the radiation emitter 103 has moved is not possible from the taken serial images, but it is possible when the sensor Se1 is used. Therefore, it is easy to take measures to prevent motion in the next imaging.

The positional variation data output from the sensor may be stored in association with the serial imaging data, so that the relationship between the positional variation of the radiation emitter 103 and the image may be examined later.

As a result, at the time of an image preview after imaging or diagnosis using a dynamic image, it is possible to confirm whether or not the motion of the radiation emitter 103 has effect by comparison with the motion of the radiation source 34.

In addition, determination of whether or not the position of the radiation emitter 103 has varied and storage of the positional variation data may be performed not only during serial imaging, but also during a period from completion of positioning the user and to the start of the serial imaging.

As a result, not to mention that the user receives a warning or imaging is stopped when the positional variation is detected during serial imaging, imaging may not be allowed in response to detection of positional variation before imaging. Therefore, it is possible to reliably avoid unnecessary imaging and to further prevent the subject S from being exposed to an unnecessary radiation

In addition, by arranging a sensor Se1 at each of at least two of the movable vehicle main body 101, the arm 102, and the radiation emitter 103, the positional variation of portions on respective sides of a movable portion may be detected.

In this way, it is possible to detect where the positional change occurs from the movable vehicle main body 101 to the radiation emitter 103 on the basis of the information from the multiple sensors.

In addition, on the basis of the information on which part the positional change occurs, it is possible to identify a position to which attention should be paid at the time of next imaging, and to reduce the possibility of the positional variation at the same portion.

### [Use of Stable Support Mechanism]

In the case of imaging where radiation is emitted from the radiation emitter 103 attached to the arm 102, there is a problem that the taken image has artifacts due to the motion of the arm 102 or the radiation emitter 103. In particular, the artifacts due to motion of the arm 102 and the radiation emitter 103 is severe in the image taken in serial imaging in which imaging period is long.

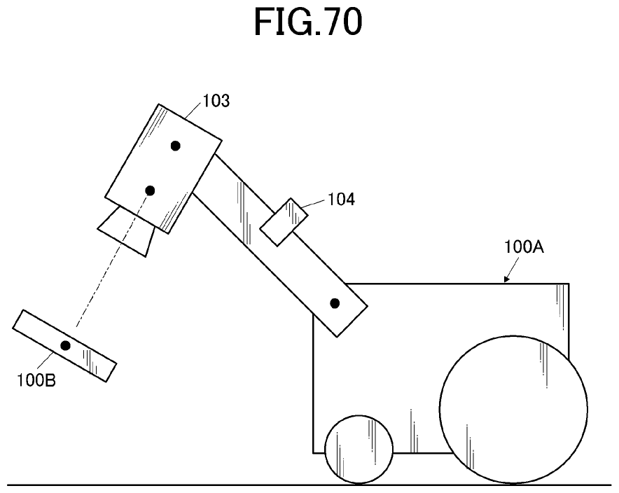

In view of such a problem, for example, as shown in FIG. 70, the system main body 100A may include a stable support mechanism 104 which changes the position of the center of gravity of the the system main body 100A.

For example, the stable support mechanism 104 may be a stabilizer.

The stable support mechanism 104 may be attached to, for example, the arm 102 or the radiation emitter 103.

In this way, the motion of the radiation emitter 103 or the arm 102 may be reduced by the stable support mechanism 104 that changes the position of the center of gravity of the system main body 100A, and an image taken with less artifact can be acquired.

[Determination based on Emission Region]

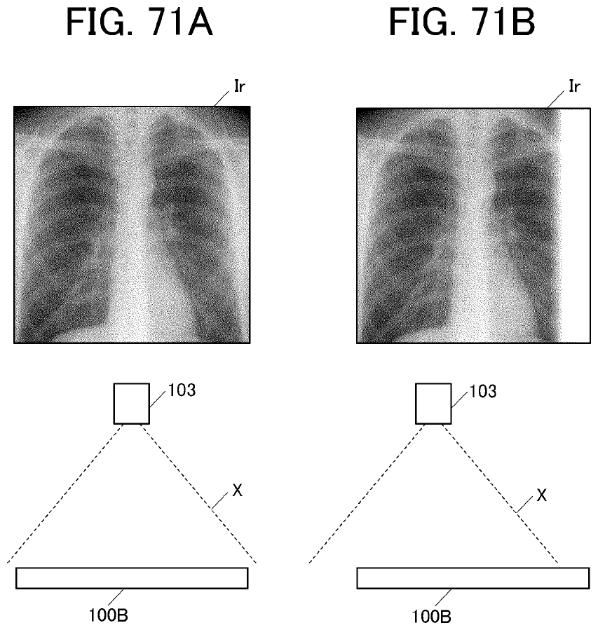

There is a problem that an error occurs in the SID or the like due to an unintended movement of the radiation source 34 during serial imaging As a result, the effective image region in the normal imaging shown in FIG. 71A varies such that a part of the imaging apparatus 100B is outside the radiation emission region as shown in FIG. 71B, for example.

In view of such a problem, it may be determined whether or not there is a region not irradiated with radiation on the basis of an image region where the radiation does not reach in the taken image. If there is a region not irradiated with radiation, at least one of the following (1) to (3) may be done.
(1) Notification to the user.
(2) Stop of radiation emission and stop of imaging.
(3) Movement of the radiation emitter 103 with the actuator 103b so that the entire imaging region is irradiated with radiation.

In this way, the entire imaging region in the acquired image can be configured to be irradiated with radiation even when the radiation source 34 has moved.

### [Attachment of Marker to Panel]

In serial imaging, the user wants to take images of only the desired motion of the subject S (for example, respiration during imaging of lung). However, the subject S may take not only the desired motion but undesired motion (for example, a body motion of moving the body vertically, horizontally, and backward or forward, etc.). In order for imaging in spite of the undesired motion of the subject S to be prevented from being continued, for example, the motion of the subject S may be detected by the motion of the marker M attached to the subject S, but it takes time to attach the marker M to the subject S.

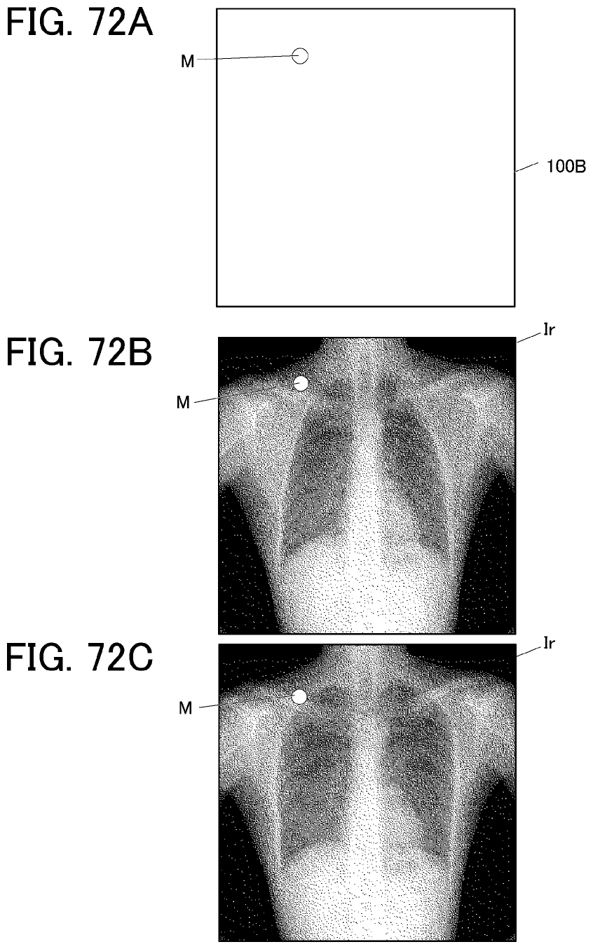

In view of such a problem, a marker M may be attached to the imaging apparatus 100B as shown in FIG. 72A, for example, and image processing etc. may be performed such that the relative movement amount of the marker M on the radiation image Ir may be extracted with respect to the outline of the body or bone as shown in FIGS. 72B and 72C.

As a result, it is possible to reduce the user's time and labor of attaching the marker M to the subject S.

The marker M may be formed of a material having high radiation transmittance. In this way, the image processing load may be reduced by viewing only the image in the region that has passed through the marker M.

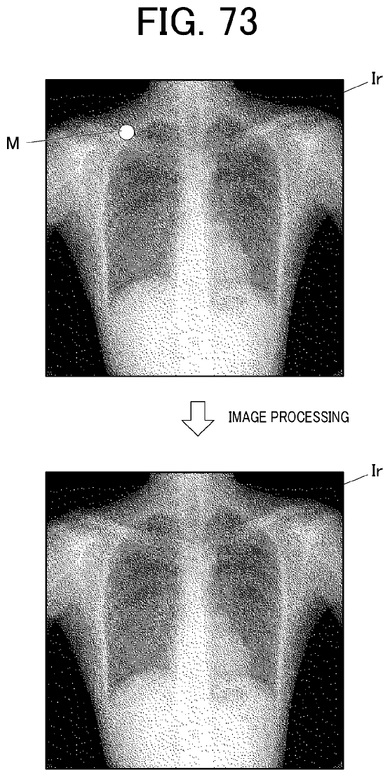

Also, for example, as shown in FIG. 73, image processing may be performed and the marker M may be deleted.

[Detection based on Marker (1)]

Further, as described above, if the marker M is provided on the imaging apparatus 100B, the backward or forward motion of the subject S (direction along the line connecting the radiation source and the imaging apparatus 100B) cannot be captured.

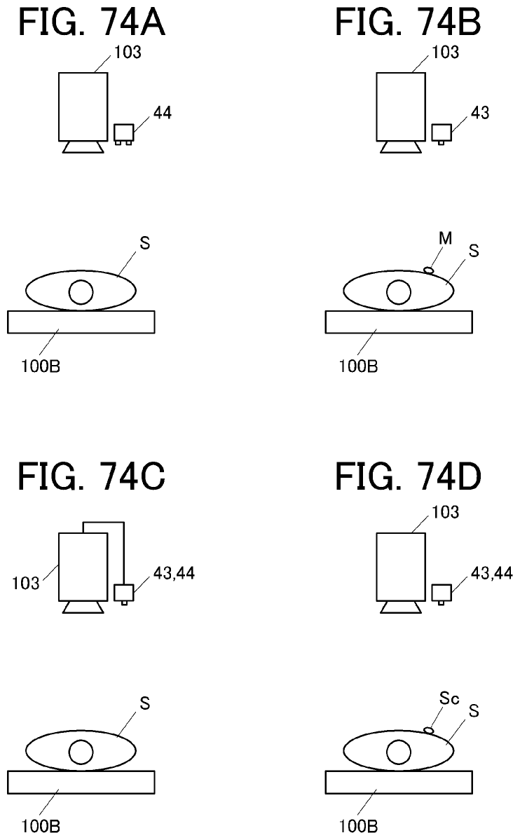

In view of such a problem, for example, as shown in FIG. 74A, a stereo camera 44 which takes an image of the subject S may be provided in the radiation emitter 103, and the body motion may be detected by automatic analysis of the image taken by the stereo camera 44. The backward or forward motion of the subject S can be captured on the basis of calculated distance to a specific portion of interest (for example, shoulder) of the subject S taken by the stereo camera 44.

Also, as shown in FIG. 74B, a marker M may be attached to the subject S and the motion thereof may be tracked with a monocular camera. In this way, the amount of body motion in the vertical and horizontal directions may be detected on the basis of the relative position of the marker M, and that in the backward or forward directions may be detected on the basis of the reduction rate of the marker M. Further, the cost and the processing amount can be reduced as compared with the case of using a stereo camera 44.

Further, as shown in FIG. 74C, the camera 43, 44 may operate in synchronization with the radiation emission timing, so that only the data taken by the camera at the emission timing may be analyzed. In this way, the data processing amount can be reduced.

As shown in FIG. 74D, the scintillator Sc may be used as the marker M, and data from camera may be acquired at the scintillator emission timing (radiation emission timing). In this way, the position of the marker M can be easily detected by the camera, even in a dark place. Further, imaging can be performed even if the system main body 100A and the camera are not synchronized.

Although the configuration using the stereo camera 44 has been described in the above, the present invention is not limited thereto. That is, the same detection can be performed using an optical camera other than a stereo camera.

Further, configuration to control the magnification of the camera depending on the emission field of the collimator 35 may be provided. By linking the emission field of the collimator 35 and the imaging region of the camera, the region subjected to radiation imaging can be taken by the camera, and it is not necessary to take image of an unnecessary region by the camera. In addition, it is possible to minimize privacy infringement due to unnecessary imaging by the camera.

On the other hand, the imaging region by the camera may be larger than the radiation emission region by the collimator 35. By making the imaging region of the camera larger than the emission region, an object that disturbs imaging can be easily found near the imaging portion with the camera.

[Detection based on Marker (2)]

In view of the problem that, if the marker M is provided on the imaging apparatus 100B, the backward or forward motion of the subject S (direction along the line connecting the radiation source and the imaging apparatus 100B) cannot be captured, an acceleration sensor or a gyro sensor may be attached to the subject S such that information can be acquired on X, Y, and Z directions and a rotation angle.

At that time, a collimator fixing device may be provided so that a specific axis of the sensor is always directed to the collimator.

In this way, it is possible to detect not only the motion of the subject S in vertical and horizontal direction but also the backward, forward, or twisting motion.

[Detection based on Marker (3)]

In serial imaging, it is desired that there is no change in the image of the subject S even when the subject S moves backward or forward.

In view of such a problem, the imaging may be performed as shown in FIGS. 74B and 74D, for example as follows. The radiation emitter 103 is provided with a camera 43 44 for imaging the subject S to whom a marker M or scintillator Sc (hereinafter referred to as a marker M etc.) is attached. The backward or forward motion and twist angle of the subject S are respectively estimated based on the enlargement ratio and deformation of the marker M etc. in the optical image taken by the camera 43, 44. The collimator may be moved (the SID, emission angle, and the vertical and horizontal positions may be adjusted) according to the estimated data.

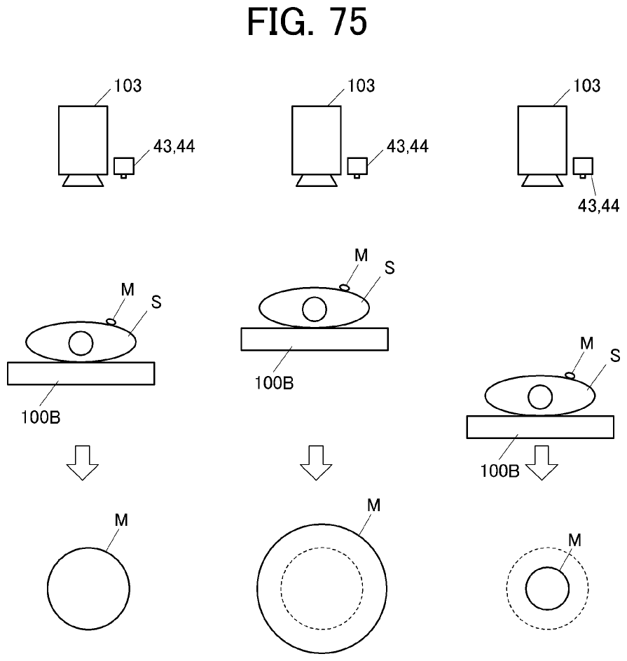

In above-described imaging, if the SID is small, the marker M etc. appears large as shown in the center of FIG. 75 compared to the one captured at an appropriate SID as shown on the left side of FIG.75. In contrast, if the SID is large, the marker M etc. appears small as shown on the right side of FIG. 75, compared to the one captured at an appropriate SID as shown on the left side of FIG.75.

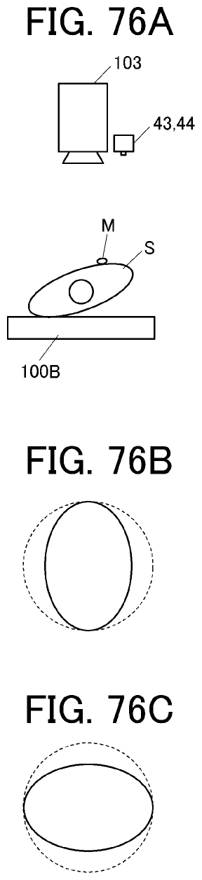

Also, when the subject S rotates left or right with the body axis as a rotation axis as shown in FIG. 76A, for example, the marker M etc. appears to be an ellipse having a major axis in the vertical direction as shown in FIG. 76B. When subject S tilts the imaging portion with respect to the body axis (for example, the subject S in a decubitus position raises the upper body), as shown in FIG. 76C, the marker M etc. appears to be an ellipse having a major axis in the horizontal direction.

In such cases, the collimator is moved so that the marker M etc. in the optical image appears to be a perfect circle of a proper size (a circle indicated by a broken line in FIGS. 76B and 76C).

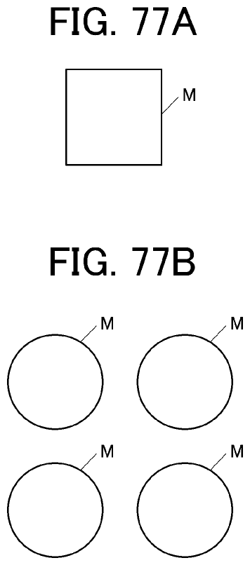

The shape of the marker M etc. may be a circle, a square, or a plurality of circles, for example, as shown in FIGS. 77A and 77B.

In this way, the image of the subject S does not change even when the subject S moves backward or forward or rotates.

The term “user” used in the embodiment for carrying out the present invention is intended to refer to, for example, a radiographer who operates the radiation imaging system and takes a radiographic image, a person who immediately checks the taken radiographic images (including the radiographer) in some operations, or a director of the radiation imaging.

In addition, it is also intended to refer to an interpreters or a doctor who adjust the taken images so that they can be easily checked, check the taken images for diagnosis, and the like.

Although embodiments of the present invention have been described and illustrated in detail, the disclosed embodiments are made for purposes of illustration and example only and not limitation. The scope of the present invention should be interpreted by terms of the appended claims

The entire disclosure of Japanese Patent Application No. 2018-168415, filed on Sep. 10, 2018, is incorporated herein by reference in its entirety.

What is claimed is:

1. A radiation imaging system comprising:
a radiation emitting apparatus having a radiation source that generates radiation;
a radiation imaging apparatus that receives radiation and generates radiation image data; and
a hardware processor, wherein
the hardware processor
detects height of the radiation source,
detects height of the radiation imaging apparatus,
calculates a distance from a focal point of the radiation generated by the radiation source to the radiation imaging apparatus, based on the height of the radiation source and the height of the radiation imaging apparatus, and
causes a display to display the distance.

2. The radiation imaging system according to claim 1, wherein
the radiation imaging apparatus includes a first air pressure sensor at a first portion, the first air pressure sensor measuring an atmospheric pressure of a height of the first air pressure sensor, and
the hardware processor detects the height of the radiation imaging apparatus based on a measurement value measured by the first air pressure sensor.

3. The radiation imaging system according to claim 2, wherein
the hardware processor stores, in a storage, a specific measurement value measured by the first air pressure sensor when the radiation imaging apparatus is arranged at a specific position, and
the hardware processor corrects the height of the radiation imaging apparatus based on the specific measurement value stored in the storage.

4. The radiation imaging system according to claim 2, wherein
the radiation emitting apparatus includes a second air pressure sensor at a specific portion, the second air pressure sensor measuring an atmospheric pressure of a height of the second air pressure sensor, and
the hardware processor corrects the height of the radiation imaging apparatus based on a measurement value measured by the second air pressure sensor.

5. The radiation imaging system according to claim 2, wherein
the radiation imaging apparatus includes a third air pressure sensor at a second portion which is different from the first portion, the third air pressure sensor measuring an atmospheric pressure of a height of the third air pressure sensor, and
the hardware processor detects a height of the first portion and detects a height of the second portion based on a measurement value measured by the third air pressure sensor.

6. The radiation imaging system according to claim 5, wherein the hardware processor calculates an incident surface inclination angle based on the height of the first portion and the height of the second portion, the incident surface inclination angle being an angle between a radiation incident surface of the radiation imaging apparatus and a predetermined plane or line.

7. The radiation imaging system according to claim 6, wherein the hardware processor calculates and outputs a difference between a radiation emission angle and the incident surface inclination angle, the radiation emission angle being an angle between an optical axis of radiation emitted from the radiation source and a predetermined surface or line.

8. The radiation imaging system according to claim 6, wherein the hardware processor calculates a height of a specific portion based on the height of the first portion and the height of the second portion, the specific portion being different from the first portion and the second portion in the radiation imaging apparatus.
